(19) United States
(12) Patent Application Publication
(10) Pub. No.: US 2025/0312516 A1
(43) Pub. Date: Oct. 9, 2025

(54) NERVE GRAFT SYSTEMS, DEVICES, AND METHODS

(71) Applicant: RENERVA, LLC, Pittsburgh, PA (US)

(72) Inventors: Lorenzo Soletti, Pittsburgh, PA (US); Anne E. Faust, Pittsburgh, PA (US); Nicole Cwalina, Roswell, GA (US); Brandon Burger, Pittsburgh, PA (US); Syndey E. Borcherding, Akron, OH (US)

(21) Appl. No.: 18/688,400

(22) PCT Filed: Sep. 30, 2022
(86) PCT No.: PCT/US2022/045365

§ 371 Date: Mar. 1, 2024

Related U.S. Application Data
(60) Provisional application No. 63/250,379, filed on Sep. 30, 2021.
(60) Provisional application No. 63/295,642, filed on Dec. 31, 2021.
(60) Provisional application No. 63/329,597, filed on Apr. 11, 2022.

(30) Foreign Application Priority Data
Sep. 8, 2021 (KR) 10-2021-0119675

(51) Int. Cl. A61L 27/36

(52) U.S. Cl. CPC A61L 27/3604; A61L 2430/40

(57) ABSTRACT

Systems, devices, and methods for treating a nerve injury in a patient are provided. A system for treating a patient includes at least one of a nerve graft-conduit or nerve cap-graft comprise a nerve segment derived from a tissue source. Methods for producing a medical device comprising a nerve segment, for obtaining an ideal degree of decellularization of a nerve segment, and for treating a patient with a medical device comprising a nerve segment are provided.

### RELATED APPLICATIONS

This application claims benefit of U.S. Provisional Patent Application Ser. No. 63/250,379 (Client Docket No. REN-006-PR1), entitled “Nerve Graft Systems, Devices, and Methods”, filed Sep. 30, 2021;
U.S. Provisional Patent Application Ser. No. 63/329,597 (Client Docket No. REN-006-PR2), entitled “Nerve Graft Systems, Devices, and Methods”, filed Apr. 11, 2022; and
U.S. Provisional Patent Application Ser. No. 63/295,642 (Client Docket No. REN-007-PR1), entitled “Neurorrhaphy Systems, Devices, and Methods”, filed Dec. 31, 2021, the content of each of which is incorporated herein by reference in its entirety for all purposes.

This application is related to:
U.S. Pat. No. 8,361,503, Issued Jan. 29, 2013;
U.S. Pat. No. 8,691,276, Issued Apr. 8, 2014;
U.S. Pat. No. 9,737,635, Issued Aug. 22, 2017;
U.S. patent Ser. No. 10/004,827, Issued Jun. 26, 2018;
U.S. patent Ser. No. 10/179,192, Issued Jan. 15, 2019;
U.S. patent Ser. No. 10/213,526, Issued Feb. 26, 2019;
U.S. patent Ser. No. 10/729,813, Issued Aug. 4, 2020; and
U.S. patent Ser. No. 10/772,989, Issued Sep. 15, 2020;
the content of each of which is incorporated herein by reference in its entirety for all purposes.

This application is related to:
U.S. patent application Ser. No. 15/996,916 (Client Docket No. REN-001-US-CON2), entitled “Extracellular Matrix-Derived Gels and Related Methods”, filed Jun. 4, 2018, published as US2019/0038803;
U.S. patent application Ser. No. 16/288,831 (Client Docket No. REN-001-US-CON3), entitled “Extracellular Matrix-Derived Gels and Related Methods”, filed Feb. 28, 2019, published as US2019/201581;
U.S. patent application Ser. No. 16/992,442 (Client Docket No. REN-002-US-CON4), entitled “Injectable Peripheral Nerve Specific Hydrogel”, filed Aug. 13, 2020, published as US2021/196860;
U.S. patent application Ser. No. 16/238,826 (Client Docket No. REN-003-US-CON1), entitled “Methods for Preparation of a Terminally Sterilized Hydrogel Derived from Extracellular Matrix”, filed Jan. 3, 2019, published as US2019/374683;
International PCT Patent Application Serial Number PCT/US2020/067431 (Client Docket No. REN-004-PCT), entitled “Extracellular Matrix Systems, Devices, and Methods of Deployment”, filed Dec. 30, 2020, published as WO2021/0138399;
U.S. patent application Ser. No. 17/788,450 (Client Docket No. REN-004-US), entitled “Extracellular Matrix Systems, Devices, and Methods of Deployment” filed Jun. 23, 2022, published as US______;
International PCT Patent Application Serial Number PCT/US2020/053570 (Client Docket No. REN-005-PCT), entitled “Extracellular Matrix Devices and Methods of Manufacture”, filed Sep. 30, 2020, published as WO2021/067456; and
U.S. patent application Ser. No. 17/762,858 (Client Docket No. REN-005-US), entitled “Extracellular Matrix Devices and Methods of Manufacture” filed Mar. 23, 2022, published as US______; the content of each of which is incorporated herein by reference in its entirety for all purposes

### TECHNICAL FIELD

The present inventive concepts relate generally to improved nerve injury treatment systems, devices, and methods.

### BACKGROUND

Peripheral nerve injuries (PNI) caused by laceration, compression, stretch, or iatrogenic injuries, such as those caused by tumor resection, have severe and wide-ranging impacts on the quality of life, productivity, and interpersonal relationships of those affected. For example, PNI in the upper extremities can prevent patients from performing basic daily activities (e.g. getting dressed, working, or feeding themselves), while facial nerve injuries can impede vocalization and are associated with social stigma and withdrawal. Existing FDA-approved nerve products are primarily indicated for use as passive support or to prevent complications (e.g. mechanical instability, neuroma, or donor site morbidity associated with autograft). None of these products has shown clinical improvement in functional outcomes.

Surgeons performing nerve repair often give their patients very poor prognoses and little hope. Nerve regeneration typically requires between 3 months and 8 months to complete and terminal functional recovery is often less than 50%.

In cases where injury is too severe to attempt surgical reconstruction of nerves or where an amputation must be performed, painful neuroma formation is a common and debilitating sequela. For example, neuroma formation is common following traumatic or oncologic nerve transections. Neuromas result from aberrant and disorganized axonal outgrowth through Schwann cell proliferation ahead of an injury site as the cells attempt to restore axonal continuity of the disrupted nerve end(s). In severe tissue damage, the regenerating axons cannot reach their target tissue and instead form a tangled bulbous mass which causes pain, likely due to pathological interactions between axons within the neuroma as well as traction between the nerve and scar tissue or ischemic necrosis of the nervous tissue.

Surgical techniques to correct neuroma formation involve burying the distal nerve ending in either bone or muscle, but the outcomes are highly variable with reports of reoperations ranging from between 40% and 81% for burial in muscle and between 33% and 91% for burial in bone with 2.8 re-interventions required on average.

There is a need for improved nerve injury and/or neuroma prevention treatment systems, devices, and methods.

### BRIEF SUMMARY

According to an aspect of the present inventive concepts, a system for treating a patient comprises at least one of a nerve graft-conduit or nerve cap-graft comprising a nerve segment derived from a tissue source. The system is configured to provide a therapeutic benefit to the patient.

In some embodiments, the at least one nerve graft-conduit or nerve cap-graft is configured to be remodeled over time into a native tissue of the patient.

In some embodiments, the at least one nerve graft-conduit or nerve cap-graft is configured to inhibit growth of tissue of the patient.

In some embodiments, the at least one nerve graft-conduit or nerve cap-graft comprises a decellularized extracellular matrix. The decellularized extracellular matrix can comprise structural and/or non-structural biomolecules. The decellularized extracellular matrix can comprise endogenous and/or exogenous growth factors. The decellularized extracellular matrix can be configured to promote and/or sustain the growth of tissue and/or associated tissue properties. The decellularized extracellular matrix can be configured to inhibit the growth of tissue and/or other associated tissue properties.

In some embodiments, the at least one nerve graft-conduit or nerve cap-graft comprises raw material harvested from a tissue source. The tissue source can comprise sensory, motor, and/or mixed nerve tissue. The tissue source can comprise autonomic nerve tissue. The tissue source can be selected and harvested from a specific animal species of a specific age, sex, and/or weight. The tissue source can be harvested from a Landrace, Landrace X, or Yorkshire pig.

In some embodiments, the at least one nerve graft-conduit or nerve cap-graft is designed and/or manufactured with one, two, or more structural and functional qualities intended to match or mismatch the structural and functional qualities of a nerve site in the patient.

In some embodiments, the at least one nerve graft-conduit or nerve cap-graft comprise different degrees of decellularization.

In some embodiments, the at least one nerve graft-conduit or nerve cap-graft is configured regenerate nerve tissue following an injury.

In some embodiments, the at least one nerve graft-conduit or nerve cap-graft is configured inhibit nerve growth following an injury.

In some embodiments, the at least one nerve graft-conduit or nerve cap-graft comprises a degradation rate in vivo of between 24 hours and 6 months.

In some embodiments, the nerve cap-graft is configured to be at least partially placed over one, two, or more nerve endings.

In some embodiments, the nerve graft-conduit is constructed and arranged as a nerve connector configured to align and/or connect two or more nerve endings.

In some embodiments, the nerve graft-conduit is constructed and arranged to at least partially replace and/or supplement one, two, or more nerves.

In some embodiments, the at least one nerve graft-conduit or nerve cap-graft is configured to exhibit one, two, or more cell adhesion properties selected from the group consisting of: integrins; laminin; immunoglobulins; cadherins; selectins; and combinations thereof.

In some embodiments, the nerve graft-conduit comprises a lumen surrounded by a luminal wall and a conduit wall. The conduit wall can comprise one or more design variables selected from the group consisting of: porosity; pore size; pore interconnectedness; pore alignment; degradation rate; swell ratio; and combinations thereof.

According to another aspect of the present inventive concepts, a method for producing a device comprising a nerve segment comprises: harvesting and/or preparing a nerve segment; decellularizing the harvested nerve segment; providing external support to the decellularized nerve segment; lyophilizing the externally supported nerve segment; creating one, two, or more desired features through the nerve segment; decellularizing and/or performing other chemical, physical, and/or mechanical treatments to the lyophilized nerve segment; stabilizing the treated nerve segment; packaging the stabilized nerve segment within a container; sterilizing the container comprising the packaged nerve segment; and shipping and/or storing the container comprising the packaged nerve segment.

According to another aspect of the present inventive concepts, a method for obtaining an ideal degree of decellularization of a nerve segment comprises: defining one, two, or more features of a desired nerve segment; selecting a nerve segment having one, two, or more the desired features and/or beginning at a known distance from a branching point; defining a desired degree of tissue processing and/or decellularization of the nerve segment; setting one, two, or more tissue processing and/or decellularization parameters; analyzing the degree of tissue processing and/or decellularization of the nerve segment during and/or following the tissue processing and/or decellularization.

According to another aspect of the present inventive concepts, a method for treating a patient comprises: deploying a device comprising at least one of a nerve graft-conduit or nerve cap-graft at a deposit site in the patient, wherein the device is configured to provide a therapeutic benefit at a treatment site.

According to another aspect of the present inventive concepts, a system for producing and deploying a medical device comprising a nerve segment as described in reference to the drawings.

According to another aspect of the present inventive concepts, a method for producing a medical device comprising a nerve segment as described in reference to the drawings.

According to another aspect of the present inventive concepts, a method for obtaining an ideal degree of decellularization of a nerve segment as described in reference to the drawings.

According to another aspect of the present inventive concepts, a method for treating a patient with a medical device comprising a nerve segment as described in reference to the drawings.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates a schematic view of a system for producing and deploying a medical device comprising an extracellular matrix, consistent with the present inventive concepts.

FIG. 2 illustrates a perspective view of a medical device comprising a nerve graft-conduit or nerve cap-graft, consistent with the present inventive concepts.

FIG. 3 illustrates a cutaway side view of a nerve graft-conduit or nerve cap-graft graft, consistent with the present inventive concepts.

FIGS. 4A-E illustrate cutaway side views of a nerve graft-conduit or nerve cap-graft graft implanted at a nerve injury site, consistent with the present inventive concepts.

FIGS. 5A-C illustrate perspective views of a nerve graft-conduit or nerve cap-graft graft, connecting a nerve ending with muscle tissue, consistent with the present inventive concepts.

FIG. 6 illustrates a method for producing a nerve graft-conduit or nerve cap-graft graft from tissue, consistent with the present inventive concepts.

FIG. 7 illustrates a method for harvesting and/or preparing nerve tissue for further manipulation, consistent with the present inventive concepts.

FIGS. 8 and 8A illustrate methods for decellularizing a nerve segment, consistent with the present inventive concepts.

FIG. 9 illustrates a method for inserting a nerve segment into a support assembly, consistent with the present inventive concepts.

FIG. 10 illustrates a method for lyophilizing a nerve segment contained within a support assembly, consistent with the present inventive concepts.

FIG. 11 illustrates a method for creating channels through a nerve segment, consistent with the present inventive concepts.

FIG. 12 illustrates a method for decellularizing a nerve graft-conduit or nerve cap-graft is illustrated, consistent with the present inventive concepts.

FIG. 13 illustrates a method for stabilizing a nerve graft-conduit or nerve cap-graft, consistent with the present inventive concepts.

FIG. 14 illustrates a method for packaging a nerve graft-conduit or nerve cap-graft, consistent with the present inventive concepts.

FIG. 15 illustrates a method for an irradiation based sterilization of a container comprising a nerve graft-conduit or nerve cap-graft consistent with the present inventive concepts.

FIG. 16 illustrates a method for shipping and/or storing a container comprising a nerve graft-conduit or nerve cap-graft, consistent with the present inventive concepts.

FIG. 17 illustrates a method for deploying a device comprising a nerve graft-conduit or nerve cap-graft consistent with the present inventive concepts.

FIG. 18 illustrates a perspective view of a sizing tool, consistent with the present inventive concepts.

FIG. 19 illustrates a graphical representation of the rehydration of a nerve graft-conduit or nerve cap-graft, consistent with the present inventive concepts.

FIGS. 20A and B illustrate photographs of unprocessed and processed nerve tissue, respectively, consistent with the present inventive concepts.

FIGS. 21A and B illustrate photographs of unprocessed and processed nerve tissue, respectively, consistent with the present inventive concepts.

FIGS. 22A and B illustrate photographs of an untrimmed nerve branch system and trimmed nerve segments, consistent with the present inventive concepts.

FIGS. 23A and B illustrate photographs of a nerve segment prepared for insertion into a nerve jacket, consistent with the present inventive concepts.

FIGS. 24A and B illustrate photographs of a nerve segment inserted into a nerve jacket, consistent with the present inventive concepts.

FIGS. 25A and B illustrate photographs of a nerve graft-conduit or nerve cap-graft implanted at a nerve injury site, consistent with the present inventive concepts.

FIG. 26 illustrates a photograph of an alignment assembly positioned below a material removal assembly, consistent with the present inventive concepts.

FIG. 27 illustrates a schematic view of container to hold, transport, and/or treat one or more nerve segments, consistent with the present inventive concepts.

FIG. 28 illustrates a perspective view of a support assembly for manufacturing a nerve graft-conduit or nerve cap-graft, consistent with the present inventive concepts.

FIGS. 29A-E illustrate histological images of native nerve tissue versus decellularized nerve tissue and assay results for the decellularized nerve tissue, consistent with the present inventive concepts.

FIGS. 30A-F illustrate a generalized nerve jacketing process, consistent with the present inventive concepts.

FIGS. 31A-D illustrate scanning electron microscopy (SEM) images for morphological assessment of nerve graft-conduits following lyophilization in tubing with various porosities, consistent with the present inventive concepts.

FIGS. 32A-C illustrate photographs of a nerve graft-conduit lumen creation, or nerve cap-graft socket creation consistent with the present inventive concepts.

FIGS. 33A-G, illustrate in-vitro and histological images of a nerve graft-conduit, consistent with the present inventive concepts.

FIGS. 34A and B illustrate photographs of a nerve graft-conduit and a nerve cap-graft following implantation at a sciatic nerve injury, respectively, consistent with the present inventive concepts.

FIGS. 35A-H illustrate histological images of a neuroma control, a nerve cap-graft at 8 weeks post-injury in a rodent model, consistent with the present inventive concepts.

FIGS. 36A-T illustrate results demonstrating nerve tissue maintaining multiple structure and functional components following decellularization, consistent with the present inventive concepts.

FIGS. 37A and B illustrate a photograph and graphical representation of a gel derived from porcine sciatic nerve promoting Schwann cell (SC) proliferation and axon regrowth at the site of a nerve injury, respectively, consistent with the present inventive concepts.

FIG. 38 illustrates a graphical representation of a nerve graft-conduit comprising a peripheral nerve matrix, consistent with the present inventive concepts.

FIG. 39 illustrates a graphical representation of a nerve cap-graft comprising a peripheral nerve matrix, consistent with the present inventive concepts.

FIG. 40 illustrates a table containing feature development parameters of a nerve cap-graft, consistent with the present inventive concepts.

FIG. 41 illustrates a table containing feature development parameters of a nerve graft-conduit, consistent with the present inventive concepts.

FIG. 42 illustrates a table containing nerve graft-conduit or nerve cap-graft feature creation strategies, consistent with the present inventive concepts.

FIG. 43 illustrates a table containing nerve graft-conduit or nerve cap-graft matrix morphology control strategies, consistent with the present inventive concepts.

FIG. 44 illustrates a table containing nerve graft-conduit or nerve cap-graft clinical requirements, consistent with the present inventive concepts.

FIGS. 45 through 53 illustrate various graphical representations, photographs, histological images, and graphs regarding the development of an acellular nerve cap-graft xenograft for neuroma prevention.

FIGS. 54A-D illustrate various theories of operation, framework, and timeline for both nerve graft-conduit or nerve cap-graft, consistent with the present inventive concepts.

FIG. 55 illustrates a method for obtaining an ideal degree of decellularization in order to obtain desired structure and function in a nerve graft-conduit or nerve cap-graft, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer, or section. Thus, a first limitation, element, component, region, layer, or section discussed below could be termed a second limitation, element, component, region, layer, or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

As used herein, the term “functional element” is to be taken to include one or more elements constructed and arranged to perform a function. A functional element can comprise a sensor and/or a transducer. In some embodiments, a functional element is configured to generate and/or deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

As used herein, the term “fluid” can refer to a liquid, gas, gel, or any flowable material, such as a material which can be propelled through a lumen and/or opening.

It is appreciated that certain features of the present inventive concepts, which are, for clarity, described in the context of separate embodiments, may also be provided in combination in a single embodiment. Conversely, various features of the present inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the present inventive concepts have been simplified to focus on elements that are relevant for a clear understanding of the present inventive concepts, while eliminating, for purposes of clarity, other elements that those of ordinary skill in the art will appreciate may also comprise a portion of the present inventive concepts. However, because such elements are well known in the art, and because they do not necessarily facilitate a better understanding of the present inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are improved nerve injury treatment systems, devices, and methods.

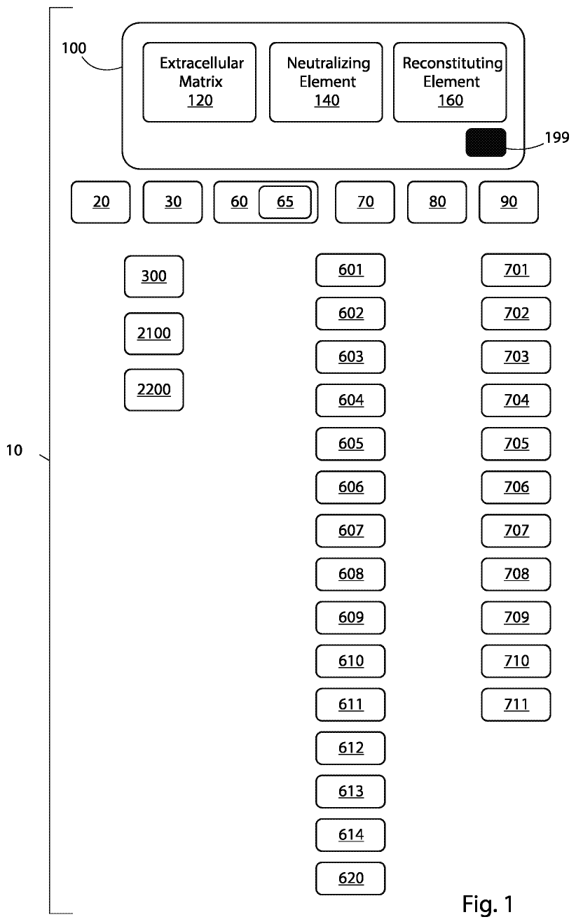

Referring now to FIG. 1, a schematic view of a system for producing and deploying a medical device comprising an extracellular matrix is illustrated, consistent with the present inventive concepts. System 10 comprises medical device 100 shown, as well as various components used to manufacture, package, sterilize, and/or deploy device 100. Device 100 is configured to be deployed (e.g. injected, inserted, delivered, implanted, and the like) at one, two, or more “deposit sites”, such as to provide a therapeutic benefit at one, two, or more “treatment sites”. Each treatment site can comprise a location that is proximate to and/or remote from the associated deposit site. In some embodiments, a treatment site comprises a location that is relatively the same location as the associated deposit site. Device 100 can be deployed at the deposit site to promote, and/or otherwise support, tissue growth of a patient (e.g. support tissue growth and/or regeneration at locations proximate and/or remote from the deposit site). In some embodiments, device 100 is remodeled over time into native tissue of the patient. In some embodiments, device 100 is deployed at the deposit site to inhibit growth of patient tissue to prevent one, two, or more conditions derived from such tissue growth. As used herein, the deposit site can comprise one, two, or more locations on and/or within the patient.

Device 100 comprises a decellularized extracellular matrix, ECM 120 shown. ECM 120 can comprise structural and non-structural biomolecules, including, but not limited to, collagens, elastins, laminins, glycosaminoglycans, proteoglycans, antimicrobials, chemoattractant, cytokines, matrix bound vesicles, and endogenous (i.e. naturally present in ECM 120) and/or exogenous (i.e. added to one, two, or more components of the device 100) growth factors. ECM 120 can be configured to promote and/or sustain the growth of tissue and/or associated tissue properties (e.g. structural proteins, growth factors, etc.) proximate to and/or remote from the deposit site. ECM 120 can be configured to inhibit the growth of tissue and/or other associated tissue properties (e.g. structural proteins, growth factors, etc.) proximate to and/or remote from the deposit site. ECM 120 can be derived, or otherwise produced, from one, two, or more raw material 65 as described herein. In some embodiments, ECM 120 is derived from a raw material as described in applicant's co-pending International PCT Patent Application Serial Number PCT/US2020/053570, entitled “Extracellular Matrix Devices and Methods of Manufacture”, filed Sep. 30, 2020. ECM 120 can comprise a concentration of native protein between 2 mg/mL and 50 mg/mL, such as a concentration between 5 mg/mL and 20 mg/mL, such as a concentration of approximately 10 mg/mL. The protein concentration can be configured to improve a parameter of ECM 120, such as to improve solubility, reconstitution, solution viscosity, solution injectability, solution mixability, gelation kinetics, working time, and gel mechanical/structural properties, such as storage modulus, stiffness, suture retention strength, microstructure, porosity, pore size distribution, liquid or gas permeability, cell penetration, cell phenotype, gene expression, protein expression, degradation rate, and/or durability.

Device 100 can further comprise a neutralizing element 140 and/or a reconstituting element 160, each configured to interact (e.g. physically, chemically interact) with ECM 120. In some embodiments, neutralizing element 140 and/or reconstituting element 160 interact with ECM 120 to cause a physical, mechanical, and/or chemical change to ECM 120 and/or other component of system 10. Neutralizing element 140 can be configured to counteract, or otherwise offset, a property (e.g. physical, mechanical, chemical property) of ECM 120, reconstituting element 160, and/or other component of system 10. Neutralizing element 140 can comprise an element selected from the group consisting of: water; buffer solutions, such as phosphate buffer solution (PBS), buffer amino acids (e.g. proline and histidine), or Tris buffers; base solutions, such as sodium hydroxide (NaOH); and combinations of these. Neutralizing element 140 can be configured to extend its ability to function as a neutralizing element over time (i.e. neutralizing element 140 shelf life). In some embodiments, the alteration of the buffer concentration of neutralizing element 140 prevents the natural pH drift over time of the solution due to the interaction with environmental CO₂. In some embodiments, ECM 120 comprises a fluid and neutralizing element 140 comprises a concentration of PBS that is configured to modify (e.g. increase, decrease) the mechanical strength of ECM 120, modify (e.g. increase, decrease) a gelation time of ECM 120, modify (e.g. increase, decrease) a gelation kinetics of ECM 120; and/or modify (e.g. increase, decrease) a gelation temperature of ECM 120. In some embodiments, neutralizing element 140 comprises a buffer solution in water with the components and ranges of concentration shown hereinbelow in Table 1:

TABLE 1
Buffer Solutions

| | | |
|---|---|---|
| Buffer/Neutralizing | | Preferred Conc (g/L) |
| Component | Range Conc (g/L) | 7.5-15 mg/mL |

| | | | |
|---|---|---|---|
| NaOH | 0.1-3.0 | g/L | 0.5-1.01 |
| KCl | 0.01-1.0 | g/L | 0.22 |
| NaH₂PO₄ | 0.1-1.5 | g/L | 0.60 |
| NaCl | 0.3-9.0 | g/L | 3.51 |
| Na₂HPO₄ | 0.1-10.0 | g/L | 2.84 |

Reconstituting element 160 can be configured to change, or otherwise modify, a property (e.g. physical, chemical, mechanical, biological, and/or shelf-life property) of ECM 120, neutralizing element 140, and/or other component of system 10. For example, neutralizing element 140 comprising PBS can affect physical, chemical, mechanical, and/or biological properties of ECM 120. Reconstituting element 160 can comprise water. In some embodiments, reconstituting element 160 and neutralizing element 140 are combined to comprise a single reconstituting and neutralizing solution.

Raw material 65 can comprise sensory, motor, and/or mixed nerve tissue. In some embodiments, raw material 65 comprises autonomic nerve tissue. In some embodiments, raw material 65 comprises spinal cord nerve tissue. In some embodiments, raw material 65 comprises ventral and/or dorsal root ganglion. In some embodiments, raw material 65 comprises somatic nerve tissue. In some embodiments, raw material 65 comprises sciatic nerve tissue, such as bilateral sciatic nerves. In some embodiments, raw material 65 comprises brachial plexus tissue. Tissue harvested from multiple (e.g. two, three, or more) nerve types can be pooled to provide a larger quantity and/or heterogenous raw material 65. In some embodiments, raw material 65 is selected and harvested from a specific animal species of a specific age, sex, and/or weight, and from a specific anatomic location within the peripheral nervous system in order to produce one or more desired features in device 100. In some embodiments, this specific anatomic location includes a specific nerve branch, as well as a specific position within a specific nerve branch (e.g. a position at a defined distance from a prior and/or neighboring nerve branch). Desired features of device 100 can include, but are not limited to: structural and functional qualities relating to the overall nerve area (e.g. external diameter); structural and functional qualities of the area occupied by fascicles (e.g. number of fascicles, and/or fascicular density, such as can be measured by the area of fascicles divided by the total nerve area); structural and functional qualities of the area occupied by axons (e.g. average axonal cross-sectional area, average myelin thickness, and/or axonal density, such as can be as measured by the total axonal area divided by the total nerve cross-sectional area); and combinations of these.

In some embodiments, device 100 is designed and/or manufactured with one, two, or more structural and functional qualities intended to match or mismatch the structural and functional qualities of the nerve site at the deposit site. For example, device 100 can be intentionally designed and/or manufactured to have a similar size and/or number of fascicles as the nerve at the deposit site, in order to support nerve regeneration. As another example, device 100 may be intentionally designed and/or manufactured to mismatch fascicle number and/or fascicle area to inhibit nerve regeneration by creating a convergence (e.g. funneling excessive axonal growth into an insufficient distal space) and/or a divergence (e.g. excessive splicing of axonal growth into multiple distal recipients) in the regenerating axons.

In some embodiments, device 100 comprises nerve tissue segments that have different degrees of decellularization depending on the radial depth. During decellularization, nerve segments are submerged in a detergent solution. The radial distance from the edge of the nerve can define the mass transport of detergent solution into each specific area and the mass transport of cellular content out of the area. As a function of this difference in mass transport, the outer segments of the nerve can be exposed to more detergent solution and are subsequently more decellularized than the inner segments, resulting in a consistent gradient of cellular content (e.g. myelin) along the radial depth into the nerve segment, with the highest level of cellular content proximate the center of the nerve segment and the lowest level of cellular content proximate the outer edge of the nerve segment.

Raw material 65 can comprise tissue harvested from a tissue source 60 selected from the group consisting of: a mammal, such as pig, human, cow, horse, and the like; an amphibian, such as salamander (e.g. an axolotl) and the like; a chondrichthyan, such as shark and the like; reptile, such as chelonians, crocodiles, snakes, and the like; a cephalopod, such as squid and the like; marine invertebrate animals, such as starfish, tunicate, geoduck, and the like; and combinations of these. In some embodiments, raw material 65 comprises tissue harvested from a Landrace, Landrace X, or Yorkshire pig. For example, raw material 65 can comprise sciatic nerve tissue, such as sciatic nerve tissue harvested from a tissue source 60 comprising a pig (e.g. a male pig), such as a pig with a weight between 1 lbs and 400 lbs, such as a weight between 50 lbs and 300 lbs, such as a weight of approximately 250 lbs.

Raw material 65 can comprise tissue harvested from one, two, or more similar and/or dissimilar tissue sources 60. Tissue harvested from multiple (e.g. two or more) tissue sources 60 can be pooled to provide a larger quantity of homogeneous raw material 65. Raw material 65 can comprise tissue harvested from a uniform sex, such as tissue harvested from all male tissue sources 60 or all female tissue sources 60. Tissue harvested from a uniform sex can increase tissue consistency. Raw material 65 can comprise tissue harvested from both male and female tissue sources 60. Raw material 65 can comprise tissue harvested from an adult and/or juvenile tissue source 60, such as tissue harvested from all adult tissue sources, all juvenile tissue sources, or both adult and juvenile tissue sources. Raw material 65 can comprise tissue harvested from a genetically uniform tissue source 60 (e.g. tissue from a single genetic strain of animals). Alternatively or additionally, raw material 65 can comprise tissue harvested from a genetically modified tissue source 60. For example, raw material 65 can comprise tissue harvested from an α1,3-galactosyltransferase knockout pig.

Raw material 65 can comprise tissue harvested from one, two, or more tissue sources 60 that provide an increased potency and/or altered mechanical, physical, and/or chemical characteristics of raw material 65. Raw material 65 can comprise an increased potency and/or altered characteristic of an element selected from the group consisting of: nerve tissue type; adult tissue; juvenile tissue; tissue from genetically-modified animals or tissue transfected with genetic material; tissue from mechanically conditioned animals or mechanically conditioned tissue; tissue from chemically conditioned animals or chemically conditioned tissue; tissue from pharmacologically conditioned animals or pharmacologically conditioned tissue; tissue from physically conditioned animals or physically conditioned tissue; tissue from psychologically conditioned animals; and combinations of these.

Raw material 65 can comprise tissue harvested from one, two, or more tissue sources 60 that is subsequently subjected to a conditioning and/or other tissue regimen. The tissue regimen can be configured to modify the mechanical, physical, and/or chemical characteristics of raw material 65. In some embodiments, raw material 65 is cross-linked to alter its degradation rate and/or orient its microstructure.

Raw material 65 can comprise tissue harvested from one, two, or more tissue sources 60 comprising an animal that adhered to a pre-determined diet, exercise, chemical, pharmacological, physical, psychological stimulation, and/or other regimen. The pre-determined regimen can be configured to modify the anatomical and/or physiological characteristics of tissue source 60. In some embodiments, the animal adhered to a physical stimuli regimen, such as exercise, electrical stimulation, mechanical conditioning (e.g. stretch, compression), physical conditioning (e.g. thermal, light exposure), and radiation. In some embodiments, the applicable animal adhered to a psychological conditioning regimen, such as different levels of daily stress or lack thereof, amount of space per animal, level of socialization, different sleep/light cycles, and level of induced sexual or reproductive activity.

Device 100 can be constructed and arranged to be used in the field of nerve injury and repair including applications in which nerve regeneration is desirable (e.g. nerve transection, nerve gap injury, nerve transfer, etc.) and/or applications in which nerve growth inhibition following nerve injury is desirable (e.g. nerve/limb amputation, prevention of painful neuroma, prevention of other neuromas, etc.).

Device 100 can be constructed and arranged to be used in a therapeutic, diagnostic, and/or other clinical applications in one or more medical fields, such as dentistry (e.g. endodontics, orthodontics), dermatology, ophthalmology, obstetrics, gynecology, cardiology and cardiac electrophysiology, gastroenterology, orthopedic, oncology, neurology, neurosurgery, endocrinology, lymphology, surgery (e.g. plastic aesthetic, plastic reconstruction, otolaryngology, and oral and maxillofacial surgery), and the like. In some embodiments, device 100 is constructed and arranged as a bulking agent for administration during a surgical procedure (e.g. a plastic reconstruction, aesthetic procedure). In some embodiments, device 100 is constructed and arranged as an embolic, clotting, and/or obstructive agent for administration during a surgical, minimally invasive, and/or percutaneous procedure.

Device 100 can be constructed and arranged for treatment of male and female sexual dysfunction or for sexual enhancement applications, such as male erectile dysfunction, retarded or premature ejaculation, lack of sensation, penile enlargement, and female genital cosmetic, reconstructive, and enhancement surgery.

Device 100 can be constructed and arranged for a therapeutic and/or clinical application in a veterinary field, such as large animal surgery, small animal surgery, farm animal surgery, competitive animal conditioning, farm animal conditioning, and military and training animal conditioning.

Device 100 can be constructed and arranged for a cosmetic application, such as a tissue bulking agent, restoration of facial animation or improvement of facial expressivity. Device 100 can comprise a topical formulation configured for the treatment of skin conditions.

Device 100 can be constructed and arranged for a food supplement application, such as to improve skin health, improve hair health, improve nail health, relieve joint pain and increase motility, prevent bone loss, improve heart health, increase muscle mass, and increase physical performance.

Device 100, comprising ECM 120, can comprise a configuration selected from the group consisting of: a fluid and/or semi-fluid (either or both, “fluid” herein), such as a hydrogel, cream, ointment, or the like; a spongy material; a compressed material, such as a film; a solid material, such as a wrap, nerve conduit, nerve graft, nerve cap, suture, or the like; an aerosolized material, such as a spray; a flowable particulate, such as a micronized and flowable particulate; a powder; a fibrous material; and combinations of these. In some embodiments, device 100 is configured to deliver one, two, or more therapeutic agents (e.g. agent 70 described herein) to the patient (e.g. pharmaceutical drugs, stem cell therapies, etc.), such was when device 100 further comprises a plurality of microspheres comprising a therapeutic agent.

Device 100 can comprise a mechanical strength and/or degradation rate/durability that is modified via at least one of chemical cross-linking or physical cross-linking.

Device 100 can comprise a degradation rate in vivo of between 24 hours and 6 months, such as a degradation rate in vivo of between 2 weeks and 2 months, such as a degradation rate in vivo of approximately 4 weeks.

In some embodiments, device 100 comprises a fluid comprising a dynamic viscosity between 20 cP and 200 cP. Device 100 can comprise a lower viscosity for injectable applications, such as a viscosity of between 1 cP and 100 cP. Device 100 can comprise a greater viscosity for topical applications, such as a viscosity of between 1000 cP and 45,000 cP.

In some embodiments, device 100 comprises a fluid having an osmolarity of between 100 and 365 mOsmol/L, such as an osmolarity of between 200 and 280 mOsmol/L. The osmolarity can be adjusted to modify (e.g. increase, decrease) the mechanical properties, gelation kinetics, working time, and/or responsiveness to temperature of device 100.

In some embodiments, device 100 comprises a fluid that transitions to a gel. Device 100 can be configured to transition from a fluid to a gel via self-assembly, fibrillogenesis, chemical cross-linking, physical cross-linking, and combinations of these. Device 100 can transition to a gel prior to its deployment at the deposit site, such that device 100 transitions to a gel ex vivo. In some embodiments, device 100 is configured to transition to a gel at a time between 0.5 minutes and 2 hours prior to deployment of device 100 at the deposit site. Device 100 can transition to a gel during and/or after its deployment at the deposit site, such as transition to a gel in situ. In some embodiments, device 100 is configured to transition to a gel at a time between 0.5 minutes and 10 minutes within the time of deployment of device 100 at the deposit site. Device 100 can comprise a volume that decreases as it transitions from a fluid to a gel. For example, device 100 can contract over a period of time as it transitions from a fluid to a gel. In some embodiments, device 100 further comprises a plurality of expansion elements configured to compensate for the decrease in volume. The expansion elements can be configured to maintain a relatively constant volume of device 100, such that the expansion elements correspondingly expand as device 100 contracts.

In some embodiments, device 100 comprises a semi-fluid and/or solid that is molded, or otherwise manipulated, into a geometric shape prior to, during, and/or after deployment at the deposit site.

In some embodiments, device 100 is constructed and arranged as a coating configured to at least partially cover one, two, or more surfaces of the deposit site. Device 100 can be configured to coat a surface of the deposit site via an atomization process, such as an atomization process performed using tool 80. Device 100 can be configured to coat a surface of the deposit site via a brushing process, such as a brushing process performed using tool 80. Device 100 can be configured to coat a surface of the deposit site via a dipping process, such as a dipping process performed using tool 80.

In some embodiments, device 100 is constructed and arranged as a “nerve cap-graft” configured to be at least partially placed over one, two, or more nerve endings, such as peripheral nerve matrix cap, PNM-CAP 2200, described herein. In some embodiments, the nerve cap-graft comprises a cylindrical device open at one end and closed or open at the opposite end, and into which a nerve end can be inserted and secured. For example, device 100 can be placed over one or more nerve ending/s following an amputation. As another example, device 100 can be configured to embed a severed nerve directly into a muscle body to promote reinnervation of the muscle.

In some embodiments, device 100 is constructed and arranged as a nerve connector configured to align and/or connect two or more nerves (e.g. end-to-end; end-to-side, side-to-side nerve connections, etc.) at a treatment site. For example, device 100 can connect two or more nerve stumps and promote nerve regrowth between the nerve stumps.

In some embodiments, device 100 is constructed and arranged as a nerve conduit, graft (e.g. allograft, autograft, xenograft), or a combination of a graft and a conduit (e.g. graft-conduit herein), and can be configured to at least partially replace and/or supplement one, two, or more nerves at a treatment site. Device 100 can comprise one or more internal longitudinal tunnels, these can be intertwining tunnels constructed and arranged to mimic the natural plexus structure of a peripheral nerve.

In some embodiments, device 100 is constructed and arranged to fill the lumen of a conduit, graft, graft-conduit combination (“nerve graft-conduit” herein, and such as implant 20, and/or PNM-G 2100 described herein), or cap-graft (such as PNM-CAP 2200 described herein). Such conduit, graft, or graft-conduit can comprise polymeric materials, such as polycaprolactone, PLLA, PGA, silicone, polyurethane, PET, or PTFE. In some embodiments, such conduit or graft can comprise naturally derived materials such as collagen, elastin, GAG, keratin, chitosan, and/or combinations of synthetic and naturally derived materials. In some embodiments, device 100 is comprised of a segment of decellularized nerve, with or without modification to create one or more internal longitudinally oriented tunnels.

In some embodiments, device 100 is constructed and arranged as a suture material, such as a nerve suture material. ECM 120 can be extruded or drawn using textile manufacturing techniques to fabricate a biodegradable mono- or multi-stranded filament configured to connect or repair nerve tissues. Device 100 can be constructed and arranged as a suture material comprising a straight strength and a knot strength, such that the knot strength is between 70% and 80% of the straight strength. Device 100 can be constructed and arranged as a suture comprising a size between 6-0 and 10-0. Device 100 can be constructed and arranged as a suture comprising a tensile strength greater than 0.5N, such as greater than 1N, such as greater than 2N.

In some embodiments, device 100 is constructed and arranged as an electrode coating. Device 100 can be configured to reduce inflammation, infection, scarring, and/or facilitate nerve growth and/or integration at the tissue-electrode interface of an implantable device. Growth and/or integration of nerve tissue at the tissue-electrode interface can reduce impedance and improve conductivity, such as to improve signal to noise reductions. Reduction of inflammation and/or scarring can prolong the useful life of the electrode, lead, or sensor. In some embodiments, device 100 is configured to enhance the performance of a nerve stimulation device.

In some embodiments, device 100 is constructed and arranged as a scaffold configured to provide structural support for cell attachment, cell migration, cell alignment, cell proliferation, cell differentiation, cell dedifferentiation, cell phenotype, cell selection, cell development, gene expression, protein expression, protein secretion, tissue alignment, and/or tissue development at a treatment site.

Device 100 can be incorporated into (e.g. embedded in, combined with, used in conjunction with, and the like) an existing medical device and/or material. In some embodiments, device 100 is incorporated into a patch and/or film. In some embodiments, device 100 is incorporated into suture material. In some embodiments, device 100 is incorporated into an adhesive, such as fibrin glue.

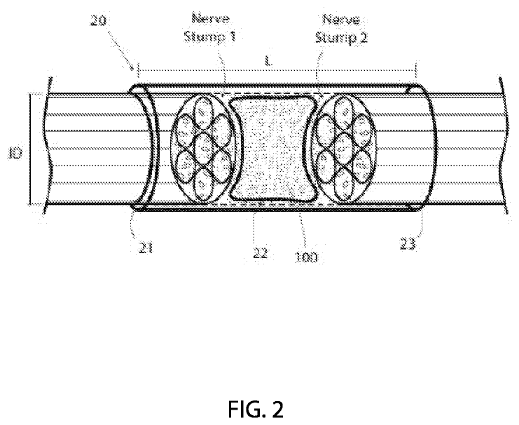

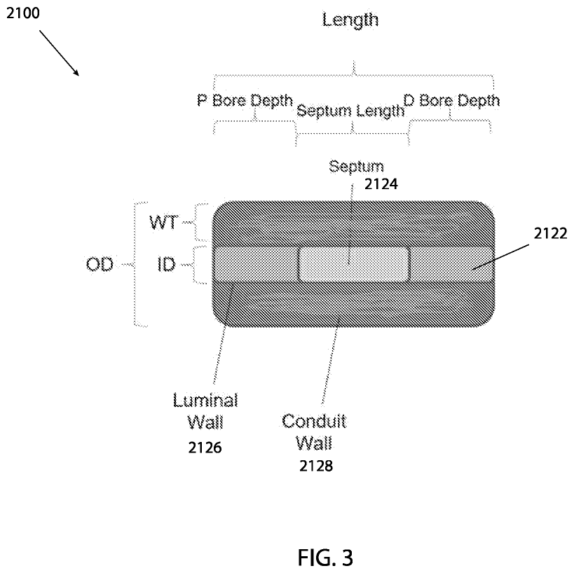

Device 100 (e.g. ECM 120) can be incorporated into a nerve graft-conduit or nerve cap-graft, such as implant 20 as described herein in reference to FIG. 2, or such as PNM-G 2100 as described herein in reference to FIG. 3. In some embodiments, device 100 is incorporated into the nerve conduit prior to implantation into a patient, such as during a manufacturing process of the nerve conduit and/or ECM 120. For example, ECM 120 comprising neutralizing element 140 can be injected into the lumen of the nerve conduit. Alternatively or additionally, the nerve conduit can be submerged in a bath of ECM 120 comprising neutralizing element 140. Alternatively or additionally, the nerve conduit can be spray coated with ECM 120 comprising neutralizing element 140. Subsequently, the nerve conduit comprising ECM 120 and neutralizing element 140 can be dehydrated via vacuum drying or lyophilization. Prior to implantation, the dehydrated nerve conduit can be rehydrated upon immersion into a saline solution. As another example, ECM 120 comprising a lyophilized cake can be press-fit into the nerve conduit, such as when ECM 120 comprises a micronized lyophilized cake. As another example, device 100 can be injected via a needle into the lumen of the nerve conduit and allowed to gel at a temperature of approximately 37° C. prior to implantation into the patient. As another example, the nerve conduit can be impregnated with device 100 via a vacuum source. The nerve conduit can be held within a channel and the vacuum source can be applied at one end of the conduit while device 100 is injected at an opposite end. Device 100 can be configured to permeate (e.g. transmural permeation) through the nerve conduit. As another example, the nerve conduit can be impregnated with device 100 via submersion into a bath comprising device 100. As another example, the nerve conduit can be impregnated with device 100 via a spray coating comprising device 100. As another example, the nerve conduit can be inserted into a container comprising device 100 and centrifuged or otherwise agitated within the container. As another example, the nerve conduit can be impregnated with device 100 via immersion into a fluid flow comprising device 100. As another example, the nerve conduit can be printed with device 100.

In some embodiments, device 100 is incorporated into a conduit or into a nerve via percutaneous or minimally invasive methods. For example, device 100 can be injected via a needle under ultrasound or fluoroscopy guidance into the implanted nerve conduit, such as injected into the lumen of the nerve or conduit via an end and/or through a side wall of the nerve or conduit.

Device 100 can be delivered, injected, implanted, and/or otherwise deployed (“deployed” herein) proximate a treatment site. Device 100 can be deployed into, onto, and/or at the deposit site, such as a focal area of a treatment site.

Device 100 can be deployed to extend to, or otherwise cover, one, two, or more locations beyond the deposit site (e.g. into locations of the treatment site or other locations). Device 100 can be deployed to extend longitudinally beyond the deposit site. In some embodiments, device 100 extends proximally from the deposit site, such as between 2 mm and 20 mm proximally from the deposit site, such as between 2 mm and 5 mm, such as between 5 mm and 10 mm, such as between 10 mm and 20 mm. In some embodiments, device 100 extends distally from the deposit site, such as between 2 mm and 20 mm distally from the deposit site, such as between 2 mm and 5 mm, such as between 5 mm and 10 mm, such as between 10 mm and 20 mm.

Device 100 can be deployed at one, two, or more deposit sites positioned about the circumference of a nerve within and/or externally to the nerve. Two or more deposit sites can comprise a uniform spacing about the circumference of the nerve. The two or more deposit sites can comprise a non-uniform spacing about the circumference of the nerve. For example, device 100 can be deployed at a first deposit site representing 0°, at a second deposit site that is 120° relative to the first deposit site, and at a third deposit site that is 240° relative to the first deposit site (and 1200 relative to the second deposit site).

Device 100 can be deployed at one, two, or more deposit sites about the circumference of a nerve and can be further deployed at one, two, or more locations beyond the deposit sites, as described herein. In some embodiments, deployment of device 100 at the deposit sites and locations beyond the deposit sites comprise a matrix of device 100 along the external surface of the nerve.

In some embodiments, the deposit site comprises a location (e.g. one, two, three, or more locations) within the central nervous system, such as a site located within and/or surrounding the brain and/or spinal cord.

In some embodiments, the deposit site comprises a location within the peripheral nervous system, such as a site located outside the brain and spinal cord, including any location along and/or around the peripheral nervous system spanning from the dorsal and/or ventral root ganglia to motor, sensory, or autonomic endings (e.g. end-muscle plate, Pacinian corpuscle, Ruffini endings). In some embodiments, the deposit site comprises a location within, and/or around, and/or proximate an uninjured nerve. In some embodiments, the deposit site comprises a location within, and/or around, and/or proximate a diseased nerve. In some embodiments, the deposit site comprises a location within, and/or around, and/or proximate a nerve injury, such as an intra-nerve and/or peri-nerve injury location. In some embodiments, the deposit site comprises a location within, and/or around, and/or proximate a partial or full nerve transection, such as a transected and repaired nerve (e.g. epineural and/or fascicular repair, such as neurorrhaphy). In some embodiments, the deposit site comprises the location of a nerve amputation (for example, following a limb amputation with which two or more nerves are left amputated and each of such nerve stumps represents a potential deposit site). For example, device 100 can be deployed to provide an interface between two or more nerves, nerve stumps, or nerve epineural windows. The two or more nerve stumps can be coapted together, such as via a suture or fibrin glue configured to eliminate or otherwise reduce a gap length between the nerve stumps. Alternatively, the two or more nerve stumps are not coapted together and a calculated gap length is maintained between the nerve stumps. The calculated gap length can be configured to promote nerve cone sprouting and alignment from a proximal nerve stump having a greater degree of freedom to properly align toward a distal nerve stump. In some embodiments, the deposit site comprises a location within, around, and/or proximate a nerve injury repaired with a nerve transfer technique, such as an end-to-end transfer, side-to-side transfer, end-to-side transfer, or supercharged end-to-side transfer. In some embodiments, the deposit site comprises a location within, around, and/or proximate a nerve crush injury, such as an acutely crushed nerve. In some embodiments, the deposit site comprises a location within, around, and/or proximate a nerve stretch injury, such as an acutely stretched nerve. In some embodiments, the deposit site comprises a location within, around, and/or proximate a compression nerve injury, such as chronic compression with or without prior surgical release. In some embodiments, the deposit site comprises a location within, around, and/or proximate a nerve gap injury repaired with a nerve conduit or graft, such as an allograft, autograft, conduit, or guide. In some embodiments, the deposit site comprises a location within, around, and/or proximate a targeted muscle reinnervation. For example, device 100 can be deployed to promote total muscle reinnervation. In some embodiments, the deposit site comprises a location within, around, and/or proximate a neurotization. For example, device 100 can be deployed to promote direct muscle neurotization, such that device 100 can be deployed within, around, and/or proximate a nerve apposed into and/or onto muscle, such as to promote nerve sprouting into the muscle. For example, device 100 can be deployed to promote combined muscle neurotization, such that device 100 can be deployed within, around, and/or proximate a nerve apposed into and/or onto muscle via a nerve conduit, such as to promote nerve sprouting into the muscle. In some embodiments, the deposit site comprises a location within, around, and/or proximate a nerve end surgically connected to muscle and configured to reactivate the muscle, such as proximate an end-to-end nerve transfer or Oberlin procedure. In some embodiments, the deposit site comprises a location within, around, and/or proximate a nerve attached to isolated patches of muscles, such as directly via direct neurotization or via existing innervation (e.g. regenerative peripheral nerve interface). In some embodiments, the deposit site comprises a location within, around, and/or proximate a nerve end surgically connected to muscle and configured to create an electrical activation map on the muscle surface, such as proximate a total muscle reinnervation. For example, the electrical activation map can be detected by skin electrodes configured to control electronic prostheses. In some embodiments, the deposit site comprises a location within, around, and/or proximate a nerve biopsy. In some embodiments, the deposit site comprises a location within, around, and/or proximate an iatrogenic (e.g. medically, surgically induced) nerve injury. In some embodiments, the deposit site comprises a location within, around, and/or proximate a brachial plexus injury. For example, device 100 can be deployed during an Oberlin or Leechavengvongs procedure. In some embodiments, the deposit site comprises a location within, around, and/or proximate a peripheral nerve injury in which there is no nerve gap. In some embodiments, the deposit site comprises a location within and/or proximate a peripheral nerve injury in which a gap closure is achieved with or without flexion of an extremity. In some embodiments, the deposit site comprises a location within, around, and/or proximate a tissue burn, such as a superficial burn, a partial thickness burn, and/or a full thickness burn. In some embodiments, the deposit site comprises a location within, around, and/or proximate a truncated nerve following an amputation. For example, device 100 can be deployed at the site of one or more nerves affected by an amputation. In some embodiments, the deposit site comprises a location that contributes to a symptom attributed to a migraine. For example, device 100 can be deployed to treat, or otherwise alleviate, migraine symptoms. In some embodiments, the deposit site comprises a location within, around, and/or proximate muscle tissue. In some embodiments, the deposit site comprises a location within, around, and/or proximate denervated muscle tissue. For example, device 100 can be deployed to promote neuromuscular junction formation. In some embodiments, the deposit site comprises a location within, around, and/or proximate a nerve end surgically connected to a sensory receptor, such that device 100 can be deployed to promote nerve sprouting into the receptor and/or reestablish sensation. In some embodiments, the deposit site comprises distributed locations via topical applications onto a surgically created cavity, onto a mucosal surface or cavity, into an organ, and/or onto the skin or injured skin surface.

In some embodiments, device 100 is deployed into a deposit site comprising muscle tissue. In some embodiments, device 100 is deployed into a deposit site comprising tendon, ligament, or cartilage tissue. In some embodiments, device 100 is deployed into a deposit site comprising organ tissue. In some embodiments, device 100 is deployed into a deposit site comprising vascular wall tissue. In some embodiments, device 100 is deployed into a deposit site comprising lymphatic tissue. In some embodiments, device 100 is deployed into a deposit site comprising neural tissue. In some embodiments, device 100 is deployed into and/or through skin tissue (e.g. epidermis, dermis, subcutaneous) proximate the deposit site. In some embodiments, device 100 is deployed topically onto the skin, organ, and/or surgical cavity. In some embodiments, device 100 is deployed into or onto the deposit site percutaneously and/or minimally invasively.

In some embodiments, device 100 is deployed into a deposit site comprising oral tissue (e.g. oral mucosa, teeth, tooth pulp, cranial nerve, tongue). In some embodiments, device 100 is deployed into the tooth root following a root canal or pulpectomy procedure. In some embodiments, device 100 is deployed into and/or around the cranial nerves. In some embodiments, device 100 is deployed into the oral mucosa. In some embodiments, device 100 is deployed into the tongue or lips.

In some embodiments, device 100 is deployed into a deposit site comprising heart nervous system including both the heart conduction system (e.g. sino-atrial node, atrio-ventricular node, Purkinje fibers, etc.) and the heart nervous system tissue from the lower cervical to the upper thoracic ganglia (e.g. cardiac plexus, parasympathetic and sympathetic nerve fibers, vagal cardiac nerve, vagus nerve, etc.).

In some embodiments, the treatment site comprises at least a portion of a nerve and device 100 is deployed at a deposit site comprising at least a portion of a neural location selected from the group consisting of: intra-mesoneurium; peri-mesoneurium; intra-epineurium; peri-epineurium; sub-epineurium; intra-fascicular, sub-endoneurium; and combinations of these.

In some embodiments, device 100 is deployed at a deposit site distal to and/or proximal to the treatment site, such as at an ectopic site. Device 100 can be deployed into muscle tissue and/or organ tissue distal to the treatment site, such as to promote nerve growth (e.g. neurotization) towards the treatment site, such as to restore sensation and/or motor control. Device 100 can be deployed into a deposit site comprising muscle tissue and/or skin tissue (e.g. epidermis, dermis, subcutaneous) distal to the treatment site, such as to recruit, or otherwise promote the growth of, muscle fibers.

Device 100 can be deployed contemporaneously (e.g. concurrently) with one, two, or more additional treatments provided to the patient (e.g. one or more treatments deployed at the deposit site, the treatment site, and/or another patient location). In some embodiments, device 100 is deployed contemporaneously with an electrical stimulation. In some embodiments, device 100 is deployed contemporaneously with a pharmacological treatment. In some embodiments, device 100 is deployed contemporaneously with a cellular treatment. In some embodiments, device 100 is deployed contemporaneously with a structural element (e.g. sutures, conduit, wrap, glue). In some embodiments, device 100 is deployed contemporaneously with physical, occupational therapy and/or electrical, magnetic, and laser stimulation.

Device 100 can comprise one or more functional elements, functional element 199 shown. Functional element 199 can comprise a sensor and/or a transducer. In some embodiments, functional element 199 comprises a biofeedback element. For example, device 100 can further comprise a biofeedback mechanism (e.g. functional element 199) configured to provide an indication of a biological, mechanical, chemical, or electric signal at the deposit site.

In some embodiments, device 100 further comprises one or more pharmacological or other agents, agent 70 shown. Agent 70 can be included in (e.g. integrated into) device 100, or it can be provided separately. Agent 70 can comprise an antimicrobial agent. Agent 70 can comprise a color additive. Agent 70 can comprise an adhesive. In some embodiments, combination of device 100 with an adhesive is configured to reinforce a suture site and/or as a primary means of structural reconstruction of a nerve. Agent 70 can comprise an immunosuppressing agent configured to promote, or otherwise support, nerve regeneration, such as FK506. Agent 70 can comprise a chemical agent configured to modify, or otherwise effect, an immune response at the deposit site, such as when agent 70 comprises an immunomodulator (e.g. immunosuppressant). Agent 70 can comprise an antimicrobial agent configured to kill, or otherwise stop or slow down, the growth of microorganisms at the deposit site, such as when agent 70 comprises one, two, or more agents selected from the group consisting of: disinfectant; antiseptic; antibiotic; and combinations of these. Agent 70 can comprise a visual additive, such as a visual additive configured to provide visibility to device 100, such as a dye configured to be visualized via an imaging device 30. Agent 70 can comprise a lubricating substance configured to minimize friction at the deposit site. Agent 70 can comprise a conductive agent configured to increase electrical conductivity at the deposit site. Agent 70 can comprise one or more anti-adhesive agents configured to prevent an adhesion at the deposit site, such as to prevent fibrotic adhesions. Agent 70 can comprise an anesthetic and/or pain reliever agent configured to induce an insensitivity at the deposit site (e.g. insensitivity to pain and/or sensation). Agent 70 can comprise a hemostatic agent configured to promote hemostasis at the deposit site. Agent 70 can comprise an antidote configured to counteract a poison and/or toxin at the deposit site, such as an antidote configured to counteract a nerve agent. Agent 70 can comprise an anti-inflammatory agent configured to reduce inflammation at the deposit site. Agent 70 can comprise a chemoattractant configured to attract motile cells to the deposit site, such as a motile cell selected from the group consisting of: Schwann cells; macrophages; endothelial cells; progenitor cells; and combinations of these. Agent 70 can comprise an agent configured to promote the production of angiogenic factors at the deposit site, such as an angiogenic factor selected from the group consisting of: angiogenin; growth factors, such as fibroblast growth factors, transforming growth factors; lipids; and combinations of these. Agent 70 can comprise an agent configured to promote cell migration, development, and/or maturation at the deposit site, such as a nerve growth factor.

In some embodiments, device 100 is configured to exhibit cell adhesion properties configured to interact and/or attach to neighboring cells at the deposit site, such as one, two, or more cell adhesion properties associated with cell-adhesion molecules selected from the group consisting of: integrins; laminin; immunoglobulins; cadherins; selectins; and combinations of these. In some embodiments, device 100 is configured to exhibit cell signaling properties configured to communicate and/or coordinate cell actions at the deposit site, such as one, two, or more properties associated with cell signals selected from the group consisting of: intracrine signals; autocrine signals; juxtracrine signals; paracrine signals; endocrine signals; and combinations of these. In some embodiments, device 100 is configured to exhibit pharmacological and/or biological properties configured to support the local microenvironment at the deposit site, such as to promote immunomodulatory action, revascularization, cell chemotaxis, cell development, nerve tissue deposition, ECM 120 or tissue remodeling, nutrient transfer, and/or waste removal. In some embodiments, device 100 is configured to increase remodeling, vascularization, and/or innervation of a nerve, nerve graft, or nerve conduit.

Device 100 can be configured to respond to electrical, mechanical, physical, and/or chemical factors, such as factors internal and/or external to device 100. For example, device 100 can be configured to respond to temperature, sound waves, electromagnetic waves, and/or light (e.g. coherent light, such as laser). In some embodiments, device 100 is configured to undergo a physical change upon the application of an ultraviolet light internally and/or externally to ECM 120, such as when device 100 transitions from a fluid to a semi-fluid and/or solid. In some embodiments, device 100 is configured to release an agent (e.g. agent 70, such as a chemical, pharmaceutical, and/or other agent) upon the application of an external vibration to device 100.

### System 10 Components

System 10 can further comprise one or more implants, implant 20 shown. Implant 20 can comprise a conduit as described herein in reference to FIG. 2. Implant 20 can comprise a graft-conduit (e.g. PNM-G 2100) or cap-graft (e.g. PNM-CAP 2200) as described herein.

System 10 can further comprise one or more imaging devices, device 30 shown, which can be configured to visualize an object (e.g. device 100). Device 30 can comprise an imaging device selected from the group consisting of: microscope, such as a surgical microscope; loupes; magnifying lens; device that provides virtual and/or augmented reality visualization; device that provides stereo visualization; device that provides infrared near-infrared visualization; device that provides thermal imaging; medical imaging device, such as an X-ray, a fluoroscope, an MRI, a CT scanner, OCT, an ultrasound, an endoscope; device that images using UV light; device that images using polarized light; device that images using fluorescent light; and combinations of these.

System 10 can further comprise one or more coating tools, tool 80 shown, which can be configured to coat a surface (e.g. a surface of a deposit site), such as an atomization tool, a brush or brushing tool, and/or a dipping tool, as described herein. Tool 80 can comprise a tattoo machine configured to deliver ECM 120 at a defined depth of a surface. Tool 80 can comprise a jet injector configured to deliver ECM 120 via high pressure at a defined depth of a surface. Tool 80 can comprise a bobbin including a coiled strand or ribbon embedded with ECM 120 configured to wind and/or canvas around a surface. Tool 80 can comprise an adhesive or adhesive strip configured to affix ECM 120 to a surface.

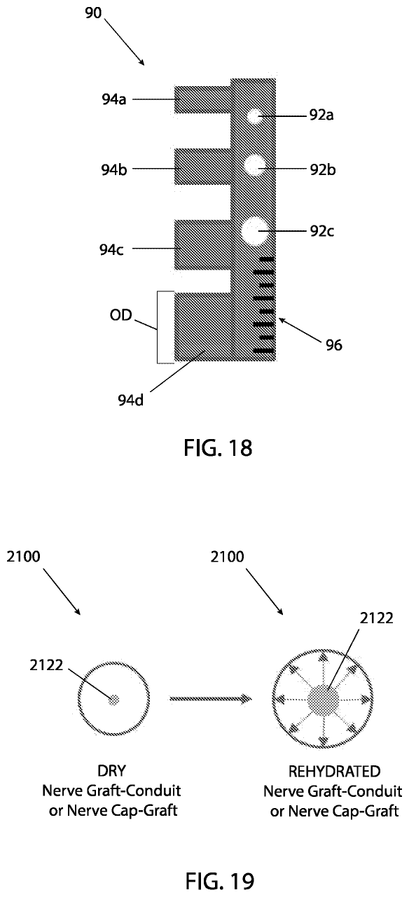

System 10 can further comprise one or more sizing tools, tool 90 shown, which can be configured to measure one or more parameters of implant 20 (e.g. PNM-G 2100, PNM-CAP). Sizing tool 90 can be configured to measure the diameter and/or length of implant 20 (e.g. PNM-G 2100, PNM-CAP 2200) as described hereinbelow in reference to FIG. 18. Sizing tool 90 can be configured to measure the diameter and/or length of one or more features of device 100 (e.g. the socket 2210 diameter and/or length of PNM-CAP 2200, the lumen diameter and/or length of PNM-G 2100). Sizing tool 90 can be configured to measure one or more parameters of the deposit site, such as the length of a nerve gap length or the diameter of a nerve stump.

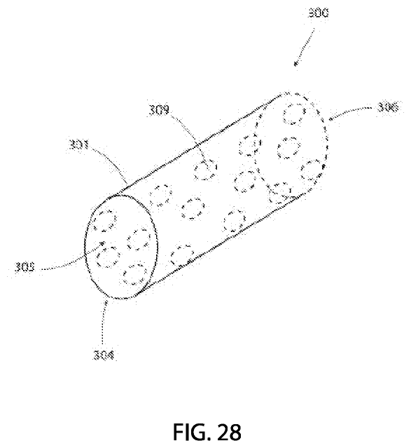

System 10 can further comprise one or more support assemblies, assembly 300 shown, which can be configured to receive and/or secure an object (e.g. implant 20, ECM 120, PNM-G 2100, PNM-CAP 2200). Support assembly 300 can be constructed and arranged as described herein in reference to FIG. 28.

System 10 can further comprise one or more environmental chambers, chamber 601 shown. Chamber 601 can comprise a temperature-controlled environmental chamber configured to chill and/or freeze an object (e.g. raw material 65) through non-cyclic and/or cyclic refrigeration. In some embodiments, chamber 601 consists of frozen ice or synthetic ice packs within an insulated container. In some embodiments, chamber 601 consists of a refrigerator or deli case with or without an incorporated shaker system.

System 10 can further comprise one or more vessels, vessel 602 shown, which can be configured to store an object (e.g. raw material 65). Vessel 602 can comprise a vented container configured to comprise one or more openings to allow for the passage of air, gas, and/or liquid through vessel 602. In some embodiments, vessel 602 is configured to store a tissue sample during tissue processing, embedding, and/or sectioning. In some embodiments, vessel 602 is configured to store one, two, or more tissue samples during decellularization, disinfection, and/or other tissue processing (e.g. permeabilization, digestion, surface treatment, lyophilization, freezing, etc.). Vessel 602 can be configured to store and handle one, two, or more tissue samples with minimal manipulation, such as individual nerve segments. Vessel 602 can comprise a rigid or flexible material and can form a cassette, pouch, or cylinder in which one, two, or more tissue samples are stored. Vessel 602 can comprise: silicone; polyolefins, such as polypropylene; other biocompatible materials; and combinations of these.

System 10 can further comprise one or more mixing devices, device 603 shown, which can be configured to agitate a fluid disposed within a component of mixing device 603. Mixing device 603 can comprise a stirring mechanism and/or a bioreactor vessel to hold a fluid and allow chemical, physical, and/or mechanical processes to occur within mixing device 603. Mixing device 603 can be configured to agitate a fluid at a speed between approximately 50 rpm and 2,000 rpm. Mixing device 603 can be configured to maximize and/or optimize mass transport within the tissue to facilitate desired chemical, physical, and/or mechanical processes. For example, mixing device 603 can produce a turbulent flow (Re>4,000) or transitional flow (2,000<Re<4,000) configured to improve mass transport over laminar flow (Re<2,000). A temperature within mixing device 603 can be increased and/or cyclic mechanical deformation can be applied to the tissue similarly to improve mass transport.

Mixing device 603 can include a reaction vessel comprising a 1 liter glass beaker, a 2 liter glass beaker, a spinner flask, a bioreactor bag, or other suitable container. In some embodiments, mixing device 603 includes a stir plate and agitates a fluid by a rotating magnetic stir bar. In some embodiments, mixing device 603 includes a fixed overhead stirrer attached to an impeller configured to rotate, thereby agitating a fluid disposed within mixing device 603. In some embodiments, device 603 consists of an ultrasonic mixing device, such as a device configured to provide mechanical shock waves. In some embodiments, mixing device 603 consists of a rocking platform with variable rocking speed and rocking angle designed to agitate a fluid in a container secured to the rocking platform surface. In some embodiments, mixing device 603 consists of an orbital shaker platform designed to agitate a fluid contained within mixing device 603 at a specified speed and/or radius. In some embodiments, mixing device 603 includes a peristaltic pump that defines a set fluid flow rate through mixing device 603. In some embodiments, mixing device 603 includes a perfusion bioreactor in which a pump, heat exchanger, and/or other components can be used to force a specific steady or periodic flow pattern around or within the tissue samples.

System 10 can further comprise one or more heating devices 604, device 604 shown. Heating device 604 can be configured to warm and/or maintain the temperature of an object (e.g. raw material 65). Heating device 604 can comprise a hotplate comprising electric heating elements. In some embodiments, heating device 604 comprises an agitating hotplate (e.g. a stirring hotplate) comprising a rotating magnetic field configured to rotate a corresponding magnetic bar that is positioned in fluid proximate a surface of heating device 604. In some embodiments, heating device 604 can comprise an incubator with or without an incorporated agitation system (e.g. a system configured to shake, rock, mix, stir, and/or otherwise agitate one or more fluids and/or other materials).

System 10 can further comprise one or more laboratory instruments, instrument 605 shown, such as an instrument selected from the group consisting of: pipette, such as a serological pipette, a positive displacement pipette; forceps, such as serrated tip forceps, single tooth forceps; scalpel, such as a stainless-steel scalpel; scraper, such as a stainless-steel scraper; blade, such as a stainless-steel blade; a cutting surface, such as a polymeric cutting board; band, such as silicone band; glassware, such as a beaker, a flask, a glass bottle; graduated cylinder; balance; spatula; conical tube; funnel; temperature probe; a measuring device, such as a ruler or caliper; and combinations of these.

System 10 can further comprise one or more lyophilization devices, device 606 shown, such as a device configured to preserve a product (e.g. ECM 120) via a low temperature dehydration process. In some embodiments, lyophilization device 606 is configured to dehydrate the product residual moisture content of between 0.1% and 10%, such as a residual moisture content between 0.5% and 4%, such as a residual moisture content of less than 4% (e.g. the moisture content as measured via the Karl-Fischer moisture content test). In some embodiments, lyophilization device 606 is configured to dehydrate the product to a residual moisture content of between 0.2% and 2.5%. In some embodiments, lyophilization device 606 is configured to create directional porosity in device 100, such as longitudinal porosity, radial porosity, circumferential porosity, and/or combinations of these. The low temperature dehydration process executed by lyophilization device 606 can comprise four primary phases: freezing, annealing, primary drying (sublimation), and secondary drying (adsorption). First, the freezing phase can be configured to cool the product within lyophilization device 606 to a temperature below its triple point to ensure later sublimation, thereby preserving the product's physical form. Freezing parameters, such as rate and temperature, can be configured to influence product pore size, pore size distribution, porosity, pore isotropy/anisotropy, etc. Secondly, the annealing phase stabilizes the molecular crystalline structure to minimize disruptions during drying. During the primary drying phase a lower pressure within lyophilization device 606, increase in temperature within lyophilization device 606, can be configured to promote water sublimation until most of the water has been sublimated. Finally, the secondary drying phase can be configured to maintain or further increase the temperature of the product to promote removal of ionically-bound water molecules (e.g. break the bonds between the product and the water molecules).

System 10 can further comprise one or more lyophilization receptacles, receptacle 607 shown, which can be configured for use with lyophilization device 606 described herein. Receptacle 607 can be configured to receive a product (e.g. ECM 120) and can be placed within lyophilization device 606 for the duration of the dehydration process. Receptacle 607 can comprise a material selected from the group consisting of: aluminum; stainless steel; glass; plastic; and combinations of these. Additionally, receptacle 607 can be depyrogenated, such as to prevent contamination of the product from pathogens on receptacle 607. In some embodiments, receptacle 607 is inserted into a storage element, such as a self-sealing pouch, prior to its placement within lyophilization device 606. In some embodiments, receptacle 607 insulates one, two, or more sides of the product contained within to promote directional lyophilization. In some embodiments, receptacle 607 compresses one, two, or more regions of the product contained therein in order to create a porosity gradient across the product.

System 10 can further comprise one or more tubes, tube 608 shown, which can be configured to store an object (e.g. ECM 120). Tube 608 can include a top, or other moveable cover.

System 10 can further comprise one or more batch mills, mill 609 shown, which can be configured to grind soft, fibrous, and/or brittle products (e.g. ECM 120). Mill 609 can be configured to receive tube 608, as described herein, and grind the product within tube 608. In some embodiments, the products are first frozen or maintained in a frozen state through the use of liquid nitrogen and/or dry ice (i.e. cryogrinding).

System 10 can further comprise one or more containers, bottle 610 shown, which can be configured to store one, two, or more fluids, powders, capsules, and the like. Bottle 610 can include a top, or other moveable cover. Bottle 610 can comprise a material selected from the group consisting of: glass; plastic, such as polypropylene, polyethylene, cyclic olefin copolymer; metal, such as stainless steel; and combinations of these. In some embodiments, Bottle 610 has a volume of 0.1 L to 5 L, such as 1 L.

System 10 can further comprise one or more material removal and/or displacement devices, material device 611 shown, which can be configured to shape or otherwise alter a surface of a product. Material device 611 can be configured to perform a function selected from the group consisting of: drill; grind; rout; plane; bore; cut; ablate; and combinations of these. Material device 611 can comprise a drill press, micro drill press, dental pin drilling unit, longitudinal file, cylindrical die, laser cutter, electrosurgery unit, ultrasound cutting, hot wire cutter, and combinations of these.

System 10 can further comprise one or more tensile tester devices, device 612 shown, which can be configured to determine the tensile or compressive stresses or forces generated by the material (e.g. ECM 120) and/or suture attachment points under displacements or deformations.

System 10 can further comprise one or more alignment assemblies, assembly 613 shown. In some embodiments, alignment assembly 613 comprises a Dremel keyless chuck and/or drill bit collet configured to secure an object therein (e.g. implant 20, PNM-G 2100, PNM-CAP 2200, etc.). In some embodiments, alignment assembly 613 comprises a three-dimensional linear stage configured to allow controlled movement in X, Y, Z directions of an object therein. In some embodiments, alignment assembly 613 comprises a jig comprising the same dimensions of a device platform (e.g. material device 611, etc.) and is configured to secure an object therein (e.g. implant 20; implant 20 secured within Dremel keyless chuck, collet, etc.) in a precise location for alteration of the object by the device. In some embodiments, alignment assembly 613 includes laser alignment at the central axis to ensure concentricity of an object therein in relation to alignment assembly 613, device (material device 611, etc.), and/or other platform.

System 10 can further comprise one or more material removal assemblies, pin assembly 614 shown. In some embodiments, pin assembly 614 comprises a Dremel keyless chuck and/or drill bit collet configured to secure a pin, needle, carbide burr, drill bit, and/or other sharpened object therein. In some embodiments, the pin comprises an outer diameter of 0.7 mm. In some embodiments, the pin comprises an outer diameter between 1.5 and 3.0 mm, such as an outer diameter of 2.0 mm.

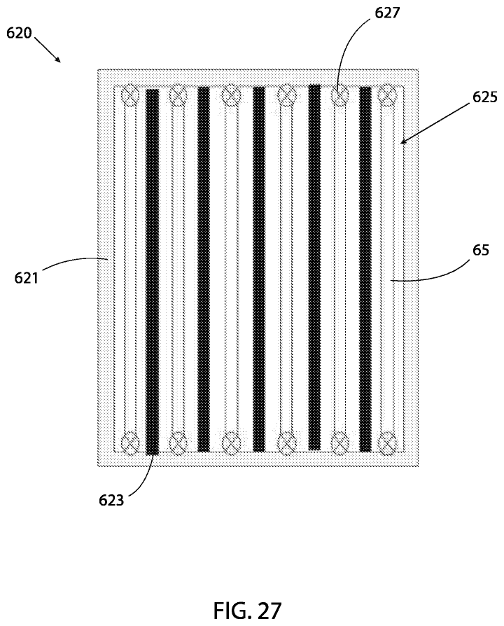

System 10 can further comprise one or more nerve holding and/or processing containers, cassette 620 shown, and as described herein in reference to FIG. 27.

System 10 can further comprise one or more buffer solutions, solution 701 shown, which can be configured to resist changes in pH when an acid and/or alkali is added to it (e.g. maintain a constant pH). In some embodiments, solution 701 comprises phosphate buffered solution or phosphate buffered saline (PBS). In some embodiments, solution 701 has a conductivity of between 1 and 30 mS/cm, such as 14 mS/cm. In some embodiments, solution 701 comprises a buffer solution in water with the components and ranges of concentration shown hereinbelow in Table 2:

TABLE 2
Buffer Solutions

| | | |
|---|---|---|
| Buffer/Neutralizing | | Preferred Conc (g/L) |
| Component | Range Conc (g/L) | 7.5-15 mg/mL |

| | | | |
|---|---|---|---|
| NaOH | 0.1-3.0 | g/L | 0.5-1.01 |
| KCl | 0.01-1.0 | g/L | 0.22 |
| NaH₂PO₄ | 0.1-1.5 | g/L | 0.60 |
| NaCl | 0.3-9.0 | g/L | 3.51 |
| Na₂HPO₄ | 0.1-10.0 | g/L | 2.84 |

System 10 can further comprise one or more cooling agents, agent 702 shown, which can be configured to reduce, and/or otherwise regulate, the temperature of a product (e.g. raw material 65). Cooling agent 702 can comprise an agent selected from the group consisting of: dry ice; dry ice with ethanol; dry ice with acetone; liquid nitrogen; wet ice; frozen ice packs; and combinations of these.

System 10 can further comprise one or more purified waters, water 703 shown, which can comprise water that has been filtered, or otherwise processed, to remove one, two, or more impurities. In some embodiments, purified water 703 comprises Type I water or water for injection.

System 10 can further comprise one or more dissociation solutions, solution 704 shown, which can be configured to dissociate adherent cells, cell aggregates, and/or tissues into single-cell suspensions. In some embodiments, dissociation solution 704 comprises a co-solution comprising 0.02% trypsin and 0.05% ethylenediaminetetraacetic acid (EDTA). Dissociation solution 704 can comprise a solution that is warmed to a temperature of approximately 35° C.

System 10 can further comprise one or more disinfecting solutions, solution 705 shown, which can be configured to destroy one, two, or more microorganisms (e.g. bacteria, virus, fungi). In some embodiments, disinfecting solution 705 comprises a co-solution comprising 0.1% peracetic acid and 4% ethanol.

System 10 can further comprise one or more detergent solutions, solution 706 shown, which can be configured to lyse and/or permeabilize cells. In some embodiments, detergent solution 706 comprises Triton X-100. In some embodiments, detergent solution 706 comprises a 4% (w/v) sodium deoxycholate solution.

System 10 can further comprise one or more sucrose solutions, solution 707 shown, which can be configured as an excipient. In some embodiment, sucrose solution 707 comprises a 1M sucrose solution.

System 10 can further comprise one or more sterile waters, water 708 shown, which can comprise water that has been processed to remove one, two, or more contaminants (e.g. bacteria, virus, fungi). In some embodiments, sterile water 708 comprises water for injection (WFI).

System 10 can further comprise one or more digestion solutions, solution 709 shown, which can be configured to break down tissue. In some embodiments, digestion solution 709 comprises a 0.01 N hydrochloric acid (HCl) solution.

System 10 can further comprise one or more digestive enzymes, enzyme 710 shown, which can be configured to break down polymeric macromolecules. In some embodiments, the digestive enzyme comprises pepsin comprising an activity level of between 0.5 U/mg and 5000 U/mg, such as an activity level of approximately 2500 U/mg. Enzyme 710 is then placed into solution 709 such that the final concentration results in an activity level of between 10 U/mL and 2500 U/mL, such as activity levels of 250 U/mL.

System 10 can further comprise one or more stabilizing and/or radioprotecting solutions, excipient 711 shown, which can be configured to provide at least one of long-term stabilization, bulking, radioprotection, heat protection, cryoprotection, increase in solubility, increase or decrease in viscosity, optimization of thermal properties for lyophilization and/or other enhancement of a product. Excipient 711 can comprise an excipient selected from the group consisting of: sucrose; ascorbic acid, glycerol, glycine, sodium ascorbate; sodium azide; vitamin E; EDTA; mannitol; glycine; Dextran; and combinations of these. Excipient 711 can be configured to increase, and/or otherwise improve, the relative solubility of a product (e.g. ECM 120). Excipient 711 can be configured to increase, or otherwise improve, the relative gelation of a product (e.g. ECM 120).

System 10 can further comprise one or more tools, tool 80 shown. Tool 80 can comprise a tool configured to coat a surface (e.g. a surface of a deposit site), such as an atomization tool, a brush or brushing tool, and/or a dipping tool, as described herein. Tool 80 can comprise a tool consisting of a tattoo machine for surface delivery of ECM 120 at a defined depth. Tool 80 can comprise a jet injector to deliver ECM 120 via high pressure at a defined depth. Tool 80 can comprise a bobbin with a coiled strand or ribbon embedded with ECM 120 wound/canvassed around the surface. Tool 80 can comprise an adhesive or adhesive strip used to affix ECM 120 to a surface.

Referring now to FIG. 2, a perspective view of a medical device comprising a nerve graft-conduit is illustrated, consistent with the present inventive concepts. Implant 20 comprises a graft-conduit (e.g. artificial, natural) configured to connect, or otherwise provide one, two, or more longitudinal channels, between two or more anatomical elements (e.g. nerve stumps). Implant 20 can comprise at least a first end 21 and at least a second end 23, with a lumen 22 therebetween. First end 21 can be constructed and arranged to receive at least a portion of a first anatomical element (e.g. first nerve stump) and second end 23 can be constructed and arranged to receive at least a portion of a second anatomical element (e.g. second nerve stump).

Lumen 22 can be configured to receive, or otherwise comprise, a therapeutic device (e.g. device 100 of the present inventive concepts), such as to maintain the relative positioning of therapeutic device between the two or more anatomical elements. Alternatively or additionally, first end 21 and/or second end 23 can be configured to receive, or otherwise comprise, a therapeutic device (e.g. device 100 of the present inventive concepts), such that therapeutic device contacts at least a portion of the anatomical elements received by first end 21, second end 23.

Implant 20 can comprise an inner diameter ID that approximates the outer diameter of at least one of the anatomical elements (e.g. a nerve stump). Implant 20 can comprise an inner diameter (ID) that is smaller than the outer diameter of at least one of the anatomical elements (e.g. a nerve stump). Implant 20 can comprise an outer diameter (OD) that approximates the outer diameter of at least one of the anatomical elements (e.g. a nerve stump). In some embodiments, implant 20 comprises an ID and/or OD that are consistent along its length. In some embodiments, implant 20 comprises an ID and/or OD that vary along its length, such as an initial IDᵢ and/or ODᵢ to adapt to the diameter of a first anatomical element and a final ID_{f} and/or OD_{f} to adapt to the diameter of a second anatomical element (not shown). In some embodiments, implant 20 comprises a funnel shape configured to allow for expansion and/or contraction of the ID and/or OD along its length. Implant 20 can comprise a length L that approximates the distance between the two or more anatomical elements. In some embodiments, implant 20 comprises a length L that is greater than the distance between the two or more anatomical elements, such as to overlap at least a portion of at least one of the anatomical elements (as shown). Alternatively or additionally, implant 20 can comprise an initial length L_{I} that is modified (e.g. trimmed or otherwise reduced) to comprise a final length L_{F}. The length of implant 20 can be modified prior to, during, and/or after the procedure in which implant 20 is implanted in the patient.

Implant 20 can comprise one, two, or more severable portions 24 (not shown) proximate first end 21 and/or second end 23. Severable portions 24 can be removed prior to, during, and/or after the procedure in which implant 20 is implanted in the patient, such as to modify the length of implant 20. In some embodiments, severable portions 24 are configured to be torn away from the remainder of implant 20. In some embodiments, severable portions 24 are configured to be cut away from the remainder of implant 20. For example, implant 20 (e.g. lumen 22) comprises a volume of device 100 that contracts after implantation within the patient. A clinician can remove at least one severable portion 24 comprising a portion of implant 20 that no longer comprises a volume of device 100 (e.g. a portion of implant 20 from which device 100 has contracted away).

Implant 20 can be configured to accept nerves in multiple orientations, such as a T-shaped implant for an end-to-side repair or a Y-shaped implant for connecting one nerve to two other nerves. In some embodiments, implant 20 comprises an angled connector configured to receive one, two, or more tissues.

Implant 20 can comprise one, two, or more materials configured to promote the growth of tissue (e.g. nerve tissue). In some embodiments, at least a portion of implant 20 comprises a material that is permeable to cells, axons, and/or nutrients (e.g. tissue cells can infiltrate the material of implant 20). In some embodiments, at least a portion of implant 20 comprises a selective permeability to pre-determined nutrients and/or cell types. In some embodiments, at least a portion of implant 20 comprises a material that supports, or otherwise promotes, tissue growth (e.g. the material comprises an agent 70 comprising growth factors). Alternatively or additionally, implant 20 can comprise one, two, or more materials configured to impede the growth of tissue (e.g. nerve tissue). In some embodiments, at least a portion of implant 20 comprises a material that is impermeable and/or otherwise resistive to cells, axons, and/or nutrients (e.g. tissue cells cannot infiltrate the material of implant 20). For example, a first portion of implant 20 can comprise a permeable material and a second portion of implant 20 can comprise an impermeable material.

Implant 20 can comprise one, two, or more materials configured to provide suture retention strength, such that implant 20 can receive one or more sutures and withstand the respective tractive force. Implant 20 can comprise a suture retention strength of no less than 0.02N, such as no less than 0.2N, such as no less 0.5N.

Implant 20 can be constructed and arranged to withstand tension, flexion, and/or torsion forces exhibited between the two or more anatomical elements (e.g. two or more nerve stumps), such as to withstand motion attributed to characteristic body and/or surrounding tissue movement. In some embodiments, implant 20 can comprise a longitudinal ultimate tensile strength great than 1 Mpa.

Implant 20 can be constructed and arranged to prevent, or otherwise resist, the formation of one or more kinks along its length. In some embodiments, implant 20 is configured to resist kink and/or compressive collapse during a bending of a joint, such as a finger or elbow. In some embodiments, implant 20 can comprise a radius of kinking curvature less than 2 cm.

Implant 20 can comprise one, two, or more materials configured to degrade at a rate compatible with the rate of tissue regeneration. Nerves can comprise a longitudinal regeneration rate between approximately 1 mm/day and 5 mm/day, and as such, implant 20 can comprise a corresponding degradation rate. For example, implant 20 comprising a length of 2 cm can be configured to degrade no earlier than 20 days after implantation. Alternatively, implant 20 can comprise a degradation rate that is slower than the longitudinal tissue regeneration rate, such as to allow for sufficient nerve volume regeneration and terminal functional recovery to occur and to prevent fibrotic and/or scar tissue formation. For example, a nerve with a gap injury of 1 cm can be expected to be crossed by growing axons from proximal to distal nerve stump in no less than 10 days (assuming a longitudinal regeneration rate of 1 mm/day). However, robust structural tissue formation between the two nerve stumps likely requires more than 20 days and full functional recovery likely requires more than one month. Therefore, the implant 20 can be configured to degrade at a slower rate than that of the nerve healing and/or regeneration process. In some embodiments, implant 20 can comprise a degradation rate that is faster at the proximal end than at the distal end, corresponding with area or tissue regeneration. For example, robust tissue formation may occur several days earlier at the proximal end of implant 20 than at the distal end. Graft-conduit degradation that is too slow may compress and damage a regenerated nerve. Therefore, implant 20 can be configured to degrade proximally before degrading distally, and at a rate matching the progression of tissue formation along the gap injury site.

Implant 20 can comprise a degradable, artificial conduit configured to break down in situ over one, two, or more periods of time. In some embodiments, implant 20 comprises a non-degradable artificial conduit, such as a conduit comprising silicone. Lumen 22 can comprise device 100 (e.g. ECM 120) comprising a hydrogel configuration, such as a degradable or non-degradable hydrogel.

Implant 20 can comprise a natural conduit harvested, or otherwise derived, from a tissue source. Implant 20 can further comprise a decellularized, natural conduit. Implant 20 can comprise a decellularized tissue that has been modified to create one, two, or more lumens (e.g. longitudinal tunnels/channels) of a controlled size. Lumen 22 can comprise device 100 (e.g. ECM 120) comprising a hydrogel configuration, such as a degradable or non-degradable hydrogel. In some embodiments, implant 20 comprises a conduit harvested, or otherwise derived, from porcine tissue (e.g. porcine nerve-tissue). In some embodiments, implant 20 comprises a composite of device 100 and one, two, or more other materials, such as a material selected from the group consisting of: polycaprolactone; PLLA; PGA; silicone; polyurethanes; PET; PTFE; decellularized tissue and/or organs; collagen; elastin; GAG; keratin; chitosan; silk; synthetic derived materials; naturally, derived materials; and combinations of these.

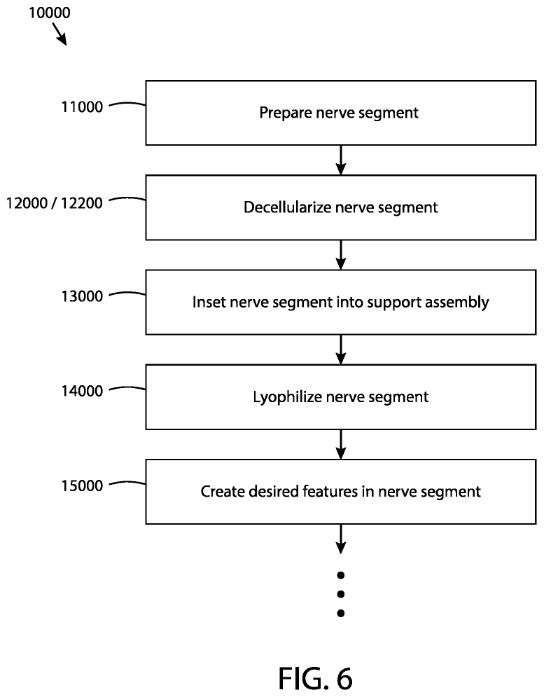

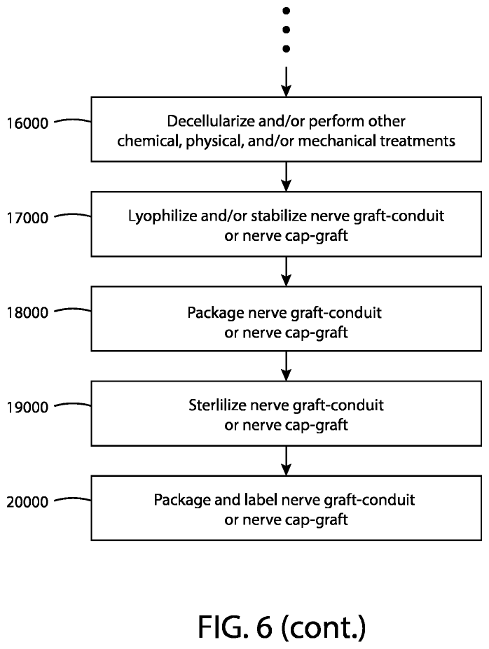

Implant 20 can comprise a conduit at least partially derived, or otherwise fabricated, from device 100 (e.g. ECM 120). Implant 20 can comprise a conduit fabricated from raw material 65 comprising a decellularized extracellular matrix (e.g. ECM 120), such as a raw material as described in applicant's co-pending International PCT Patent Application Serial Number PCT/US2020/053570, entitled “Extracellular Matrix Devices and Methods of Manufacture”, filed Sep. 30, 2020. Raw material 65 can be mechanically, physically, and/or chemically modified to create a conduit with a lumen therethrough, such as described herein in reference to Method 1000 of FIG. 6.

Implant 20 can further comprise one, two, or more cavities, spaces, and/or other reservoirs, reservoirs 25 (not shown). Reservoirs 25 can be configured to contain a volume of device 100 (e.g. ECM 120). Device 100 can be injected into implant 20 such that lumen 22 and reservoir 25 each receive a volume of device 100. As the volume of device 100 contracts within lumen 22 (e.g. contracts over time), the volume of device 100 within reservoir 25 can be pulled into lumen 22 to provide an additional supply of device 100. In some embodiments, reservoir 25 is constructed and arranged as a recess within an inner wall of implant 20. For example, device 100 can comprise a plurality of reservoirs 25 constructed and arranged along a length of implant 20. As another example, device 100 can comprise at least one reservoir 25 constructed and arranged proximate first end 21 and/or second end 23.

Implant 20 can be constructed to comprise device 100 (e.g. ECM 120) and/or can be configured to receive a volume of device 100 (e.g. ECM 120).

Implant 20 can be constructed and arranged as a nerve graft-conduit, PNM-G 2100, as described hereinbelow.

Implant can be constructed and arranged as a nerve cap-graft, PNM-CAP 2200, as described hereinbelow.

Referring now to FIG. 3, a cutaway side view of a nerve graft-conduit is illustrated, consistent with the present inventive concepts. The nerve graft-conduit comprises PNM-G 2100 as described herein. PNM-G 2100 can comprise a hybrid nerve graft-conduit derived from decellularized peripheral nerves. In some embodiments, PNM-G 2100 comprises a decellularized porcine peripheral nerve. PNM-G 2100 comprises a lumen 2122 surrounded by a luminal wall 2126 and conduit wall 2128, as shown. In some embodiments, lumen 2122 includes a central neuroinductive septum 2124. PNM-G 2100 can comprise a length comprising a proximal bore depth and distal bore depth with a septum length therebetween, as shown. PNM-G 2100 can further comprise an outer diameter OD comprising a conduit wall thickness WT and inner diameter ID, as shown.

The length of PNM-G 2100 can be controlled by one or more of the following factors: native nerve type; native nerve size; animal type from which the native nerve is harvested; animal age from which the native nerve is harvested; cutting tools employed to trim the native nerve; and mechanical stretch applied to elongate the native nerve.

The proximal and/or distal bore depth of PNM-G 2100 can be controlled by the depth of penetration via a rotating burr or drill bit during manufacturing. The proximal and/or distal bore depth of PNM-G 2100 can alternately be controlled by the speed, power, and/or number of passes of laser ablation during manufacturing. The proximal and/or distal bore depth of PNM-G 2100 can alternately be controlled by the power setting (J/s), tip size, frequency, power, current, and/or rate and regimen of tip longitudinal travel via electrosurgery or ultrasound cutting during manufacturing.

The outer diameter OD of PNM-G 2100 can be controlled by one or more of the following factors: native nerve type; native nerve size; animal type from which the native nerve is harvested; animal age from which the native nerve is harvested; use of jacketing and circumferential compression ratio; use of mechanical forces to steamroll, massage, and/or stretch the tissue; removal of material around the circumference of the tissue to reduce the OD to a desired size; application of negative hydrostatic pressure to force swelling of the tissue and increase the OD; use of chemical treatment to shrink, remove, and/or swell the tissue and change the OD; use of physical forces, such as heat, to shrink, remove, and/or swell the tissue and change the OD; and addition of material around the tissue to increase the OD, such as a membranous wrap.

The inner diameter ID of PNM-G 2100 can be controlled by the mechanical, chemical, and/or physical removal of tissue within the native nerve, such as via rotating burrs, luminal filing with abrasive thread, water cutting, ultrasound cutting or drilling, shooting of a micro bullet into the lumen, acid compounds, laser ablation, and electro-surgical methods, such as cutting via electrical current (e.g. cautery).

Septum 2124 can be constructed and arranged according to one or more design variables selected from the group consisting of: porosity; pore size; pore interconnectedness; pore alignment; degradation rate; and swell ratio (e.g. all of which can be different along the three main axes). In some embodiments, septum 2124 is created within PNM-G 2100 by leaving a portion of lumen 2122 undrilled. In some embodiments, septum 2124 is created separately and then inserted into lumen 2122 of PNM-G 2100. In some embodiments, septum 2124 is created by filling lumen 2122 with a putty, gel, and/or other fluid/semi-solid substance(s) and then solidifying the substance(s) and creating the desired macro and micro properties in situ (e.g. via lyophilization).

Luminal wall 2126 can be constructed and arranged according to one or more design variables selected from the group consisting of: porosity; pore size; pore geometry; pore interconnectedness; pore alignment; degradation rate; and swell ratio (e.g. all of which can be different along the three main axes).

Conduit wall 2128 can be constructed and arranged according to one or more design variables selected from the group consisting of: porosity; pore size; pore interconnectedness; pore alignment; degradation rate; and swell ratio (e.g. all of which can be different along the three main axes). In some embodiments, chemical and/or physical treatment are applied to shrink or swell the thickness of conduit wall 2128. The pore alignment of conduit wall 2128 can be dependent upon one or more of the following factors: leverage phase separation with defined freezing parameters (e.g. rate, temperature, etc.) to create desired crystals as template for macro porosity; lyophilization with different parameters and/or external aids (e.g. porous jacket) for water removal and generation of micro porosity; use of mechanical and/or physical forces to generate pores (e.g. laser drilling); use of chemicals to swap water content within conduit wall 2128 with chemicals to induce a controlled phase separation; use of internal or external manifolds with circumferentially distributed pores and the application of high pressure to create radially distributed channels via water cutting; and use of fiber optics with radially distributed outlets to convey laser and create radially oriented pores. The swell ratio of conduit wall 2128 can be dependent upon pore distribution, pore size, and/or directionality of pores.

Swell ratios and the associated directional components can be used to design the desired sizes of PNM-G 2100 upon hydration. For example, dry lumen 2122 diameter sizing can be utilized to obtain the hydrated (e.g. operational) lumen size. The swelling of PNM-G 2100 upon hydration can be used to create a holding force around a nerve stump, or other mechanical interactions, to anchor, align, secure, position, and/or hold PNM-G 2100 to the nerve stump. For example, a dry or partially hydrated PNM-G 2100 can be applied onto the nerve stump prior to hydration, or completion thereof, to secure lumen 2122 onto the nerve and without requiring the use of sutures. The application of a dry or partially hydrated PNM-G 2100 can be configured to foster the creation of adhesion forces upon absorbance of the moieties (e.g. via capillarity or hydrophobic/hydrophilic interactions, or other small molecular bond forces) at the deposit site within conduit wall 2128 of PNM-G 2100 and without requiring the use of sutures.

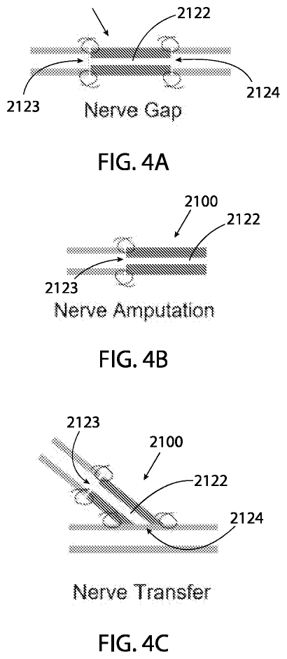

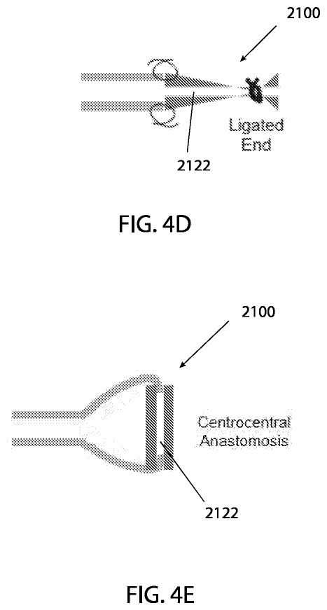

Referring now to FIGS. 4A-E, cutaway side views of a nerve graft-conduit implanted at a nerve injury site are illustrated, consistent with the present inventive concepts. The nerve graft-conduit comprises PNM-G 2100 as described herein. PNM-G 2100 can be configured for implantation at the site of a nerve injury selected from the group consisting of: nerve gap injury, such as to provide end-to-end repair; nerve transection injury, such as to provide short neurorrhaphy repair; nerve transfer, such as to provide end-to-end, end-to-side, or side-to-side repair; and nerve amputation, such as to provide nerve cap-graft and/or nerve extension to expire regenerative potential.

In some embodiments, and as shown in FIG. 4A, the deposit site comprises a nerve gap and PNM-G 2100 is configured to connect two nerve stumps. An operator can create two proximal and two distal sutures (e.g. 9-0 nylon monofilament suture) to secure PNM-G 2100 to the nerve stumps. In some embodiments, PNM-G 2100 is configured to have a larger diameter than the nerve stump, thereby creating a hole, or socket, into which an operator can insert the nerve stump, and which can be further secured with two sutures.

In some embodiments, and as shown in FIG. 4B, the deposit site comprises an amputated nerve and PNM-G 2100 is attached to an end portion of the amputated nerve. An operator can create two proximal sutures (e.g. 9-0 nylon monofilament suture) to secure PNM-G 2100 to the amputated nerve. Additionally, an operator can create a distal suture (e.g. 9-0 nylon monofilament suture) to ligate distal end 2123, as shown in FIG. 4D. Additionally, an operator can create two distal sutures to secure distal end 2123 to surrounding tissue.

In some embodiments, and as shown in FIG. 4C, the deposit site comprises a nerve transfer and PNM-G 2100 is configured to connect a healthy nerve to an injured nerve. An operator can create two proximal and two distal sutures (e.g. 9-0 nylon monofilament suture) to secure PNM-G 2100 to the healthy nerve and the injured nerve.

In some embodiments, and as shown in FIG. 4E, the deposit site comprises an amputated nerve and PNM-G 2100 is attached to an end portion of the amputated nerve. An operator can create a centrocentral anastomosis via an end-to-end suture (e.g. 9-0 nylon monofilament suture) between the fascicles of the amputated nerve, with PNM-G 2100 implanted therebetween.

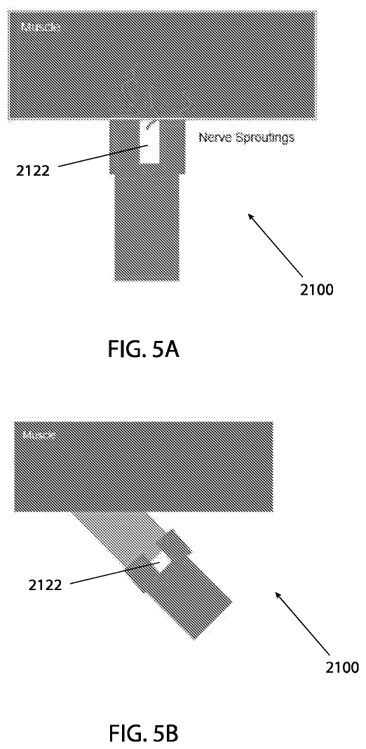

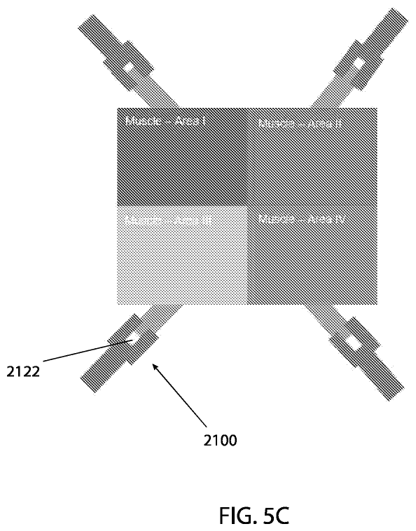

Referring now to FIGS. 5A-C, perspective views of a nerve graft-conduit connecting a nerve ending with muscle tissue are illustrated, consistent with the present inventive concepts. Implant 20 can comprise a nerve graft-conduit, PNM-G 2100, as described herein. PNM-G 2100 can be configured for implantation at the site of a nerve injury selected from the group consisting of: nerve gap injury, such as to provide end-to-end repair; nerve transection injury, such as to provide short neurorrhaphy repair; nerve transfer, such as to provide end-to-end, end-to-side, or side-to-side repair; and nerve amputation, such as to provide nerve cap-graft and/or nerve extension to expire regenerative potential. PNM-G 2100 can be configured to connect nerve endings to other tissues and organs to facilitate reinnervation (e.g. cornea, corpus cavernosum, heart, blood vessels, spinal cord, etc.).

In some embodiments, and as shown in FIG. 5A, PNM-G 2100 is implanted between muscle tissue and a nerve ending, such that the nerve ending is connected into and/or onto the muscle tissue to foster new functional connections between the nerve axonal sproutings and new and/or existing neuromuscular junctions within the muscle (e.g. direct muscle neurotization).

In some embodiments, and as shown in FIG. 5B, PNM-G 2100 is implanted between two nerve endings, one of which is connected to muscle tissue, and such that the muscle tissue is reactivated via the connected nerve endings (e.g. end-to-end nerve transfer, Oberlin Procedure, etc.)

In some embodiments, and as shown in FIG. 5C, PNM-G 2100 is implanted between two nerve endings, one of which is connected to muscle tissue, and such that an electrical activation map of the muscle tissue surface is created. The activation map can be detected from skin electrodes to control electronic prostheses (e.g. total muscle reinnervation).

Referring now to FIG. 6, a method for producing a nerve graft-conduit from tissue is illustrated, consistent with the present inventive concepts. The nerve graft-conduit can comprise PNM-G 2100 as described herein. Method 10000 comprises a sequence of sub-methods, Methods 11000, 12000, 12200, 13000, 14000, 15000, 16000, 17000, 18000, 19000, and 20000 as described herein in reference to FIGS. 6-16, respectively. Method 11000 comprises a method for harvesting and/or preparing tissue for further manipulation. Methods 12000 and 12200 comprise methods for decellularizing the tissue harvested and/or prepared in Method 11000. Method 13000 comprises a method for jacketing, or otherwise providing external support to, the decellularized tissue produced in Methods 12000 and 12200. Method 14000 comprises a method for lyophilizing the jacketed tissue produced in Method 13000. Method 15000 comprises a method for creating one, two, or more desired features (e.g. lumen 2122, socket 2210, anchoring tab 2214, etc.) through the lyophilized tissue produced in Method 14000. Method 16000 comprises a method for decellularizing and/or performing other chemical, physical, and/or mechanical treatments of nerve graft-conduit or nerve cap-graft produced in Method 15000. Method 17000 comprises a method for stabilizing a nerve graft-conduit or nerve cap-graft produced in Method 16000. Method 18000 comprises a method for packaging the nerve graft-conduit or nerve cap-graft produced in Method 17000. Method 19000 comprises a method for sterilizing the container comprising the nerve graft-conduit or nerve cap-graft produced in Method 18000. Method 20000 comprises a method for shipping and/or storing the container comprising the nerve graft-conduit or nerve cap-graft produced in Method 19000.

A method of producing a nerve graft-conduit or nerve cap-graft may include and/or omit one, two or more sub-methods as described herein. In some embodiments, the order of one, two, or more sub-methods can be altered depending on a desired feature of device 100. For example, Method 14000 may take place after Method 16000, representing lyophilization of the nerve graft-conduit or nerve cap-graft directly before stabilization and packaging.

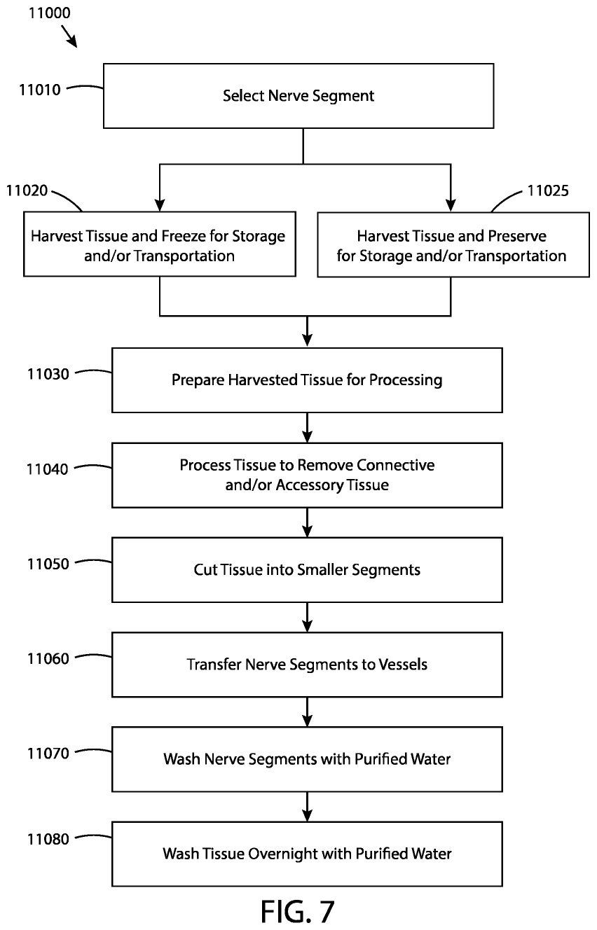

Referring now to FIG. 7, a method for harvesting and/or preparing nerve tissue for further manipulation is illustrated, consistent with the present inventive concepts. Method 11000 can be configured to harvest and/or prepare raw material 65 from tissue source 60 described herein.

In STEP 11010, raw material 65 is selected from a tissue source (e.g. tissue source 60) comprising one, two, or more desired structural and/or functional features. In some embodiments, the desired structural and/or functional features are configured to provide a desired structure and/or function to device 100 (e.g. PNM-G 2100, PNM-CAP 2200).

In STEP 11020, raw material 65 harvested from a tissue source (e.g. tissue source 60) and frozen for storage and/or transportation. Additionally, raw material 65 can be processed to remove connective and/or accessory tissue (e.g. remove non-nerve tissue). For short-term storage (e.g. for a duration less than six hours), cleaned raw material 65 can be at least partially immersed in buffer solution 701. In some embodiments, raw material 65 can be stored in chamber 601 at a temperature between approximately 2° C. and 8° C. For long-term storage and/or transportation (e.g. for a duration more than six hours), raw material 65 can be rapidly frozen in buffer solution 701. In some embodiments, raw material 65 is rapidly frozen via cooling agent 702. Raw material 65 can be stored and/or transported in chamber 601 at a temperature of approximately −80° C. (or lower temperatures such as those afforded by dry ice or liquid nitrogen storage). In some embodiments, raw material 65 is stored in chamber 601 at a temperature of approximately −80° C. for a maximum of six months. In some embodiments, raw material 65 is harvested aseptically and stored at a temperature of between 2° C. and 8° C. for up to 48 hours in a sealed environment devoid of air (e.g. to protect from oxygen) with a preservation solution comprising germostatic or germicidal agents consistent with that used for human organ procurement and transport for transplantation.

As an alternative to STEP 11020, in STEP 11025, raw material 65 is harvested from a tissue source (e.g. tissue source 60) and preserved for storage and/or transportation. Additionally, raw material 65 can be processed to remove connective and/or accessory tissue (e.g. remove non-nerve tissue). For preservation, raw material 65 can be stored in a preservation solution until proceeding to STEP 11030 described herebelow.

In STEP 11030, frozen and/or preserved raw material 65 is prepared for processing. In some embodiments, frozen raw material 65 is thawed in chamber 601 at a temperature of between 2° C. and 8° C. In some embodiments, frozen raw material 65 is thawed in chamber 601 for at least 48 hours, such as at least 72 hours. In some embodiments, preserved raw material 65 is removed from the preservation solution.

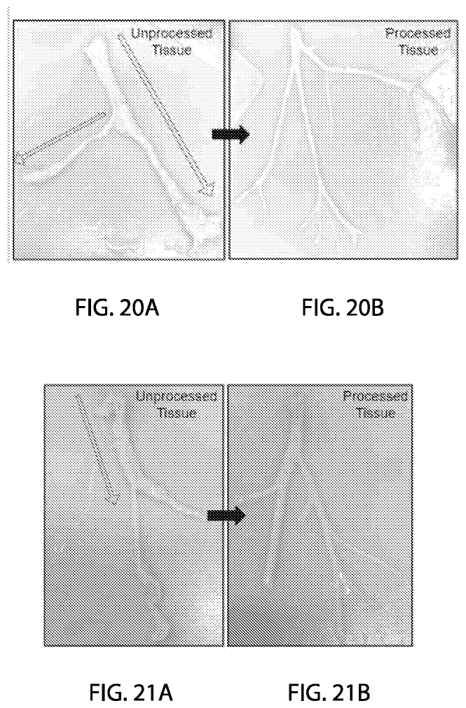

In STEP 11040, raw material 65 is further processed (e.g. cleaned) to remove additional connective and/or accessory tissue (e.g. remove non-nerve tissue), and as described hereinbelow in reference to FIGS. 20A,B and FIGS. 21A,B. Raw material 65 can be processed at a temperature of between 2° C. and 25° C.

In some embodiments, raw material 65 comprises a portion of nerve tissue comprising one, two, or more side branches. The side branches can be removed and processed as described herein.

In some embodiments, raw material 65 is processed manually (e.g. by hand, via surgical instruments such as scalpels). In some embodiments, raw material 65 is processed via a mechanical treatment using an industrial machine designed similarly to those used for peeling fruit, cleaning vegetables, or preparing meat fillet out of mammals or fish. In some embodiments, raw material 65 is processed via a chemical treatment (e.g. acid spray plus wash) to partially remove external layers of tissue from the nerve. In some embodiments, raw material 65 is processed via physical treatment involving heat, cold, radiation, laser, and/or other physical forces to ablate or otherwise remove excess tissue. In some embodiments, raw material 65 is processed via a combination of manual, mechanical, chemical, and/or physical treatments.

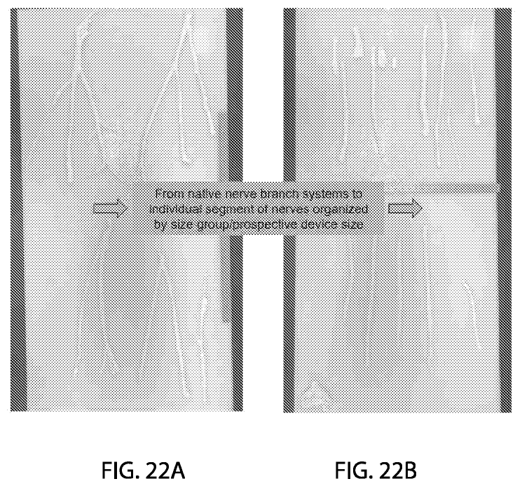

In STEP 11050, cleaned raw material 65 is cut, or otherwise divided, into smaller segments, and as described hereinbelow in reference to FIGS. 22A, B. Cleaned raw material 65 can be cut into segments comprising a length of between 0.5 cm and 20 cm, such as between 1 cm and 10 cm. Cleaned raw material 65 can comprise segments comprising a diameter of between 0.5 mm and 10 mm, such as between 1 mm and 8 mm. In some embodiments, cleaned raw material 65 is selected from a specific nerve branch and at a specific distance from the proximal branching point in order to obtain defined features, such as nerve area, fascicular density, axonal density, etc.

In some embodiments, a sizing tool 90 is used to measure the length and diameter of each segment, and as described hereinbelow in reference to FIG. 18.

In some embodiments, each segment is processed manually (e.g. by hand). In some embodiments, each nerve segment is processed via a mechanical treatment using an industrial machine designed similarly to those used for peeling fruit, cleaning vegetables, or preparing meat fillet out of mammals or fish. In some embodiments, each segment is processed via a chemical treatment (e.g. acid spray plus wash) to partially remove external layers of tissue from the nerve. In some embodiments, each segment is processed via physical treatment involving heat, cold, radiation, laser, and/or other physical forces to ablate or otherwise remove excess tissue. In some embodiments, each segment is processed via a combination of manual, mechanical, chemical, and/or physical treatments.]

In STEP 11060, raw material 65 is transferred to one, two, or more vessels 602. Cleaned raw material 65 can be transferred at a temperature of between 2° C. and 37° C. In some embodiments, each vessel 602 comprises no more than 25 g of cleaned raw material 65 and a mixing device 603 contains no more than six vessels 602. In some embodiments, each vessel 602 contains a single segment of cleaned raw material 65.

In STEP 11070, cleaned raw material 65 is washed with purified water 703. Cleaned raw material 65 can be washed at a temperature of between 2° C. and 8° C. Cleaned raw material 65 is washed with purified water 703 at least two times, such as at least three times, such as at least four times. Cleaned raw material 65 and purified water 703 can comprise a ratio between 1:20 and 1:50, such as 1:30. In some embodiments, vessel 602 is placed into mixing device 603 comprising purified water 703, such as a mixing device comprising at least 3000 mL of purified water 703. Mixing device 603 can be combined with heating device 604 configured to agitate purified water 703 at a speed between 10 rpm and 1000 rpm, such as 100±10 rpm, for at least 10 minutes, thereby washing cleaned raw material 65 within vessel 602. Purified water 703 is decanted from mixing device 603 and replaced with fresh purified water 703. Mixing device 603 is combined with heating device 604 configured to agitate purified water 703 at a speed between 10 rpm and 1000 rpm, such as 100±10 rpm, for at least 10 minutes, thereby washing raw material 65 within vessel 602 a second time. Purified water 703 is decanted from mixing device 603.

In STEP 11080, cleaned raw material 65 is washed overnight with purified water 703. Cleaned raw material 65 can be washed at a temperature of between 2° C. and 8° C. Cleaned raw material 65 and purified water 703 can comprise a ratio between 1:20 and 1:50, such as 1:30. In some embodiments, vessel 602 is placed into mixing device 603 comprising purified water 703, such as a mixing device comprising at least 300 mL of purified water 703, such as 3000 mL of purified water 703. Mixing device 603 is stored in chamber 601 at a temperature of approximately 5° C. for between 12 hours and 24 hours, such as 16 hours. During this time, mixing device 603 is combined with heating device 604 configured to agitate purified water 703 at a speed between 10 rpm and 1000 rpm, such as 100±10 rpm, thereby washing cleaned raw material 65 within vessel 602.

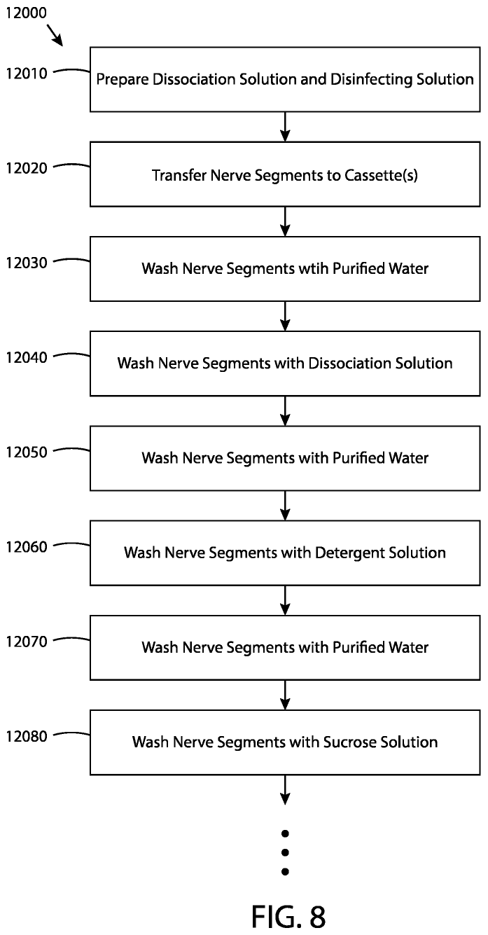

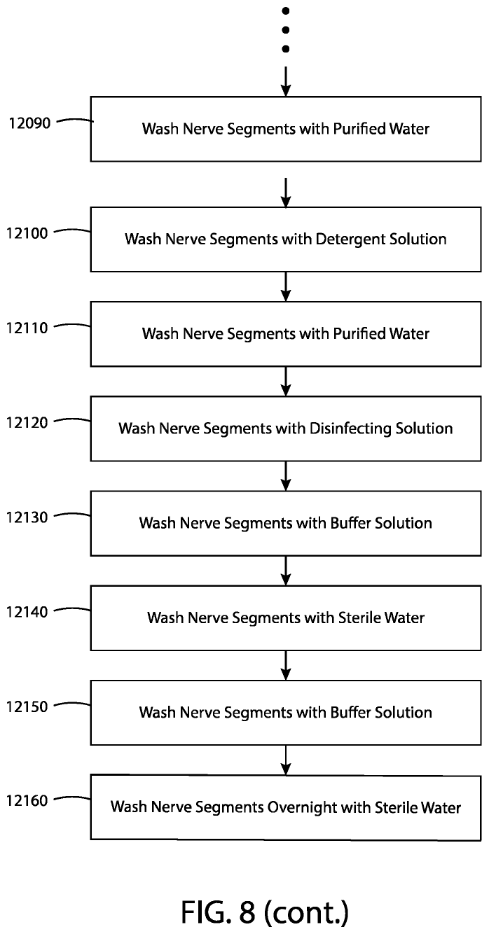

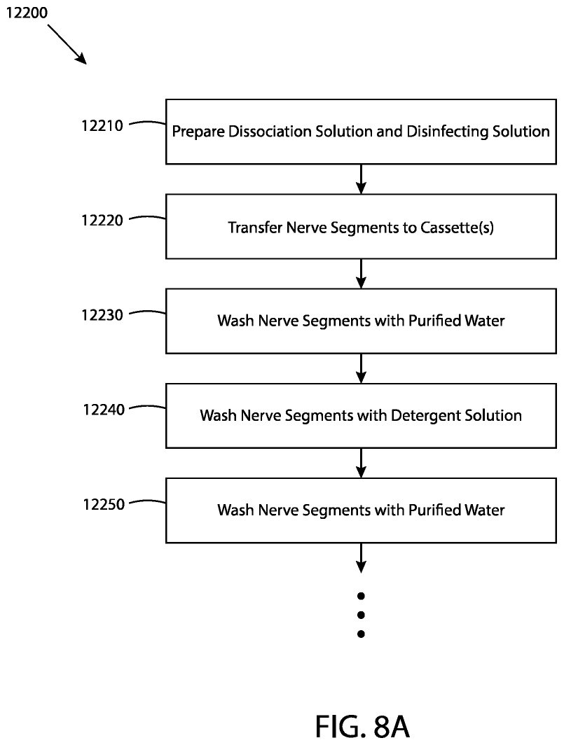

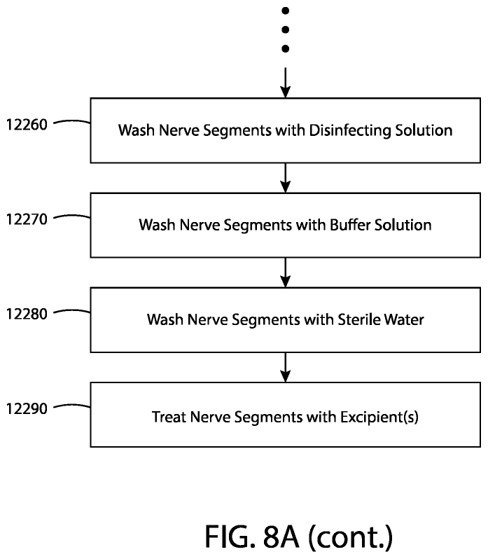

Referring now to FIGS. 8 and 8A, methods for decellularizing a nerve segment are illustrated, consistent with the present inventive concepts. Methods 12000 and 12200 can each be configured to decellularize cleaned raw material 65 harvested and/or prepared in Method 11000 described herein in reference to FIG. 7.

Referring specifically to FIG. 8, Method 12000 comprises STEPs 12010 through 12160, as described hereinbelow.

In STEP 12010, dissociation solution 704 and disinfecting solution 705 are prepared. Dissociation solution 704 and disinfecting solution 705 can be prepared at a temperature of between 2° C. and 25° C.

In STEP 12020, cleaned raw material 65 from STEP 12010 is removed from vessel 602 and transferred to one or more cassettes 620, and as described hereinbelow in reference to FIG. 27. One or more portions of raw material 65 can be anchored to cassette 620, such as to secure raw material 65 to and/or within cassette 620. In some embodiments, cleaned raw material 65 from STEP 12010 remains in vessel 602 throughout a decellularization process.

In STEP 12030, cleaned raw material 65 is washed with purified water 703. In some embodiments, purified water 703 is brought in chamber 601 to a temperature of between 2° C. and 37° C. Cleaned raw material 65 is washed with purified water 703 at least two times. Cleaned raw material 65 and purified water 703 can comprise a ratio between 1:20 and 1:50, such as 1:30. Purified water 703 is added to mixing device 603. In some embodiments, at least 300 mL of purified water 703 is added to mixing device 603. Mixing device 603 is stored in chamber 601 at a temperature of between 2° C. and 22° C. Mixing device 603 is combined with heating device 604 configured to agitate purified water 703 at 100±10 rpm, for at least 10 minutes, thereby washing cleaned raw material 65 within vessel 602. Purified water 703 is decanted from mixing device 603 and replaced with fresh purified water 703. Mixing device 603 is combined again with heating device 604 configured to agitate purified water 703 at a speed between 10 rpm and 1000 rpm, such as 100±10 rpm, for at least 10 minutes, thereby washing cleaned raw material 65 within vessel 602 a second time. Purified water 703 is decanted from mixing device 603.

In STEP 12040, cleaned raw material 65 is treated with dissociation solution 704. Dissociation solution 704 can comprise a temperature of between 2° C. and 37° C., such as 35±2° C. Cleaned raw material 65 and dissociation solution 704 can comprise a ratio between 1:20 and 1:50, such as 1:30. Dissociation solution 704 is added to mixing device 603. In some embodiments, at least 3000 mL of dissociation solution 704 is added to mixing device 603. Mixing device 603 is combined with heating device 604 configured to agitate dissociation solution 704 at a speed between 10 rpm and 1000 rpm, such as 100±10 rpm, thereby washing cleaned raw material 65 within vessel 602. Cleaned raw material 65 is washed in chamber 601 at a temperature of between 2° C. and 37° C., such as 35±2° C., for between 30 minutes and 180 minutes, such as 60±5 minutes. In some embodiments, a lipid layer forms on the surface of dissociation solution 704 and is removed using instrument 605. Dissociation solution 704 is decanted from mixing device 603.

In STEP 12050, cleaned raw material 65 is washed with purified water 703. In some embodiments, purified water 703 is pre-chilled in chamber 601 to a temperature of between 2° C. and 8° C. Cleaned raw material 65 is washed with purified water 703 at least six times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of approximately 5° C. Mixing device 603 is combined with heating device 604 configured to agitate purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least five additional times, thereby washing cleaned raw material 65 within vessel 602 at least six times.

In STEP 12060, cleaned raw material 65 is treated (e.g. washed or otherwise treated) with detergent solution 706. In some embodiments, detergent solution 706 is brought in chamber 601 to a temperature of between 2° C. and 37° C. Cleaned raw material 65 and detergent solution 706 can comprise a ratio between 1:20 and 1:50, such as 1:30. Detergent solution 706 is added to mixing device 603. In some embodiments, at least 3000 mL of detergent solution 706 is added to mixing device 603. Mixing device 603 is stored in chamber 601 at a temperature of between 2° C. and 37° C., such as approximately 20° C. Mixing device 603 is combined with heating device 604 configured to agitate detergent solution 706 at a speed between 10 rpm and 1000 rpm, such as 100±10 rpm, for between 30 minutes and 180 minutes, such as 60±5 minutes, thereby washing cleaned raw material 65 within vessel 602. Detergent solution 706 is decanted from mixing device 603.

In STEP 12070, cleaned raw material 65 is washed with purified water 703. In some embodiments, purified water 703 is brought in chamber 601 to a temperature of between 2° C. and 37° C., such as approximately 20° C. Cleaned raw material 65 is washed with purified water 703 at least twelve times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 37° C., such as approximately 20° C. Mixing device 603 is combined with heating device 604 configured to agitate purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least eleven additional times, thereby washing cleaned raw material 65 within vessel 602 at least twelve times.

In STEP 12080, cleaned raw material 65 is treated with sucrose solution 707. In some embodiments, sucrose solution 707 is pre-chilled in chamber 601 to a temperature of between 2° C. and 8° C., such as approximately 4° C. Cleaned raw material 65 and sucrose solution 707 can comprise a ratio between 1:20 and 1:50, such as 1:30. Sucrose solution 707 is added to mixing device 603. In some embodiments, at least 3000 mL of sucrose solution 707 is added to mixing device 603. Mixing device 603 is stored in chamber 601 at a temperature of between 2° C. and 8° C., such as approximately 4° C. Mixing device 603 is combined with heating device 604 configured to agitate sucrose solution 707 at a speed between 10 rpm and 1000 rpm, such as 100±10 rpm, for between 5 minutes and 60 minutes, such as 15±5 minutes, thereby washing cleaned raw material 65 within vessel 602. Sucrose solution 707 is decanted from mixing device 603.

In STEP 12090, cleaned raw material 65 is washed with purified water 703. In some embodiments, purified water 703 is brought in chamber 601 to a temperature of between 2° C. and 8° C., such as approximately 4° C. Cleaned raw material 65 is washed with purified water 703 at least six times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 8° C., such as approximately 4° C. Mixing device 603 is combined with heating device 604 configured to agitate purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least five additional times, thereby washing cleaned raw material 65 within vessel 602 at least six times.

In STEP 12100, cleaned raw material 65 is treated with detergent solution 706. In some embodiments, detergent solution 706 is brought in chamber 601 to a temperature of between 2° C. and 37° C., such as approximately 20° C. Cleaned raw material 65 and detergent solution 706 can comprise a ratio between 1:20 and 1:50, such as 1:30. Detergent solution 706 is added to mixing device 603. In some embodiments, at least 3000 mL of detergent solution 706 is added to mixing device 603. Mixing device 603 is stored in chamber 601 at a temperature of between 2° C. and 37° C., such as approximately 20° C. Mixing device 603 is combined with heating device 604 configured to agitate detergent solution 706 at a speed between 10 rpm and 1000 rpm, such as 100±10 rpm, for between 30 minutes and 180 minutes, such as 60±5 minutes, thereby washing cleaned raw material 65 within vessel 602. Detergent solution 706 is decanted from mixing device 603.

In STEP 12110, cleaned raw material 65 is washed with purified water 703. In some embodiments, purified water 703 is brought in chamber 601 to a temperature of between 2° C. and 37° C., such as approximately 20° C. Cleaned raw material 65 is washed with purified water 703 at least 18 times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 37° C., such as approximately 20° C. Mixing device 603 is combined with heating device 604 configured to agitate purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least seventeen additional times, thereby washing cleaned raw material 65 within vessel 602 at least eighteen times.

In STEP 12120, cleaned raw material 65 is treated with disinfecting solution 705. In some embodiments, disinfecting solution 705 is brought in chamber 601 to a temperature of between 2° C. and 37° C., such as approximately 20° C. Cleaned raw material 65 and disinfecting solution 705 can comprise a ratio between 1:20 and 1:50, such as 1:30. Disinfecting solution 705 is added to mixing device 603. In some embodiments, at least 3000 mL of disinfecting solution 705 is added to mixing device 603. Mixing device 603 is stored in chamber 601 at a temperature of between 2° C. and 37° C., such as approximately 20° C. Mixing device 603 is combined with heating device 604 configured to agitate disinfecting solution 705 at a speed between 10 rpm and 1000 rpm, such as 100±10 rpm, for between 30 minutes and 240 minutes, such as 120±5 minutes, thereby washing cleaned raw material 65 within vessel 602. Disinfecting solution 705 is decanted from mixing device 603.

In STEP 12130, cleaned raw material 65 is washed with buffer solution 701. In some embodiments, buffer solution 701 is brought in chamber 601 to a temperature of between 2° C. and 37° C., such as approximately 20° C. Raw material 65 and buffer solution 701 can comprise a ratio between 1:20 and 1:50, such as 1:30. Buffer solution 701 is added to mixing device 603. In some embodiments, at least 3000 mL of buffer solution 701 is added to mixing device 603. Mixing device 603 is stored in chamber 601 at a temperature of between 2° C. and 37° C., such as approximately 20° C. Mixing device 603 is combined with heating device 604 configured to agitate buffer solution 701 at speed between 10 rpm and 1000 rpm, such as 100±10 rpm, for between 5 minutes and 60 minutes, such as 15±5 minutes, thereby washing cleaned raw material 65 within vessel 602. Buffer solution 701 is decanted from mixing device 603.

In STEP 12140, cleaned raw material 65 is washed with sterile water 708. In some embodiments, sterile water 708 is brought in chamber 601 to a temperature of between 2° C. and 37° C., such as approximately 20° C. Cleaned raw material 65 is washed with sterile water 708 at least two times. Cleaned raw material 65 and sterile water 708 can comprise a ratio between 1:20 and 1:50, such as 1:30. Sterile water 708 is added to mixing device 603. In some embodiments, at least 3000 mL of sterile water 708 is added to mixing device 603. Mixing device 603 is stored in chamber 601 at a temperature of between 2° C. and 37° C., such as approximately 20° C. Mixing device 603 is combined with heating device 604 configured to agitate sterile water solution at a speed between 10 rpm and 1000 rpm, such as 100±10 rpm, for between 5 minutes and 60 minutes, such as 15±5 minutes, thereby washing cleaned raw material 65 within vessel 602. Sterile water 708 is decanted from mixing device 603 and replaced with fresh sterile water solution. Mixing device 603 is combined again with heating device 604 configured to agitate sterile water 708 at a speed between 10 rpm and 1000 rpm, such as 100±10 rpm, for between 5 minutes and 60 minutes, such as 15±5 minutes, thereby washing cleaned raw material 65 within vessel 602 a second time. Sterile water 708 is decanted from mixing device 603.

In STEP 12150, cleaned raw material 65 is washed with buffer solution. In some embodiments, buffer solution 701 is brought in chamber 601 to a temperature of between 2° C. and 37° C., such as approximately 20° C. Cleaned raw material 65 and buffer solution 701 can comprise a ratio between 1:20 and 1:50, such as 1:30. Buffer solution 701 is added to mixing device 603. In some embodiments, at least 3000 mL of buffer solution 701 is added to mixing device 603. Mixing device 603 is stored in chamber 601 at a temperature of between 2° C. and 8° C., such as approximately 4° C. Mixing device 603 is combined with heating device 604 configured to agitate buffer solution 701 at a speed between 10 rpm and 1000 rpm, such as 100±10 rpm, for between 5 minutes and 60 minutes, such as 15±5 minutes, thereby washing cleaned raw material 65 within vessel 602. Buffer solution 701 is decanted from mixing device 603.

In STEP 12160, cleaned raw material 65 is washed overnight with sterile water. In some embodiments, sterile water 708 is brought in chamber 601 to a temperature of between 2° C. and 37° C., such as approximately 20° C. Cleaned raw material 65 and sterile water 708 can comprise a ratio between 1:20 and 1:50, such as 1:30. Sterile water 708 is added to mixing device 603. In some embodiments, at least 3000 mL of sterile water 708 is added to mixing device 603. Mixing device 603 is stored in chamber 601 at a temperature of between 2° C. and 8° C., such as approximately 4° C., for between 12 hours and 24 hours, such as 16±2 hours. During this time, mixing device 603 is combined with heating device 604 configured to agitate purified water 703 at a speed between 10 rpm and 1000 rpm, such as 100±10 rpm, thereby washing cleaned raw material 65 within vessel 602. Sterile water 708 is decanted from mixing device 603.

Upon the conclusion of Method 12000, cleaned raw material 65 comprises a decellularized segmented nerve tissue (referred to as “nerve segment” herein).

Referring specifically to FIG. 8A, Method 12200 comprises STEPs 12210 through 12290, as described hereinbelow. In some embodiments, Method 12200 comprises a simplified decellularization process as described hereinabove in reference to Method 12000.

In STEP 12210, dissociation solution 704 and disinfecting solution 705 are prepared. Dissociation solution 704 and disinfecting solution 705 can be prepared at a temperature of between 2° C. and 25° C.

In STEP 12220, cleaned raw material 65 from STEP 12010 is removed from vessel 602 and transferred to one or more cassettes 620, and as described hereinbelow in reference to FIG. 27. One or more portions of raw material 65 can be anchored to cassette 620, such as to secure raw material 65 to and/or within cassette 620. In some embodiments, cleaned raw material 65 from STEP 12010 remains in vessel 602 throughout a decellularization process.

In STEP 12230, cleaned raw material 65 is washed with purified water 703. In some embodiments, purified water 703 is brought in chamber 601 to a temperature of between 2° C. and 37° C. Cleaned raw material 65 is washed with purified water 703 at least two times. Cleaned raw material 65 and purified water 703 can comprise a ratio between 1:20 and 1:50, such as 1:30. Purified water 703 is added to mixing device 603. In some embodiments, at least 300 mL of purified water 703 is added to mixing device 603. Mixing device 603 is stored in chamber 601 at a temperature of between 2° C. and 22° C. Mixing device 603 is combined with heating device 604 configured to agitate purified water 703 at 100±10 rpm, for at least 10 minutes, thereby washing cleaned raw material 65 within vessel 602. Purified water 703 is decanted from mixing device 603 and replaced with fresh purified water 703. Mixing device 603 is combined again with heating device 604 configured to agitate purified water 703 at a speed between 10 rpm and 1000 rpm, such as 100±10 rpm, for at least 10 minutes, thereby washing cleaned raw material 65 within vessel 602 a second time. Purified water 703 is decanted from mixing device 603.

In STEP 12240, cleaned raw material 65 is treated (e.g. washed or otherwise treated) with detergent solution 706. In some embodiments, detergent solution 706 is brought in chamber 601 to a temperature of between 2° C. and 37° C. Cleaned raw material 65 and detergent solution 706 can comprise a ratio between 1:20 and 1:50, such as 1:30. Detergent solution 706 is added to mixing device 603. In some embodiments, at least 3000 mL of detergent solution 706 is added to mixing device 603. Mixing device 603 is stored in chamber 601 at a temperature of between 2° C. and 37° C., such as approximately 20° C. Mixing device 603 is combined with heating device 604 configured to agitate detergent solution 706 at a speed between 10 rpm and 1000 rpm, such as 100±10 rpm, for between 30 minutes and 180 minutes, such as 60±5 minutes, thereby washing cleaned raw material 65 within vessel 602. Detergent solution 706 is decanted from mixing device 603.

In STEP 12250, cleaned raw material 65 is washed with purified water 703. In some embodiments, purified water 703 is brought in chamber 601 to a temperature of between 2° C. and 37° C., such as approximately 20° C. Cleaned raw material 65 is washed with purified water 703 at least twelve times. Cleaned raw material 65 and purified water 703 can comprise a ratio between 1:20 and 1:50, such as 1:30. Purified water 703 is added to mixing device 603. In some embodiments, at least 3000 mL of purified water 703 is added to mixing device 603. Mixing device 603 is stored in chamber 601 at a temperature of between 2° C. and 37° C., such as approximately 20° C. Mixing device 603 is combined with heating device 604 configured to agitate purified water 703 at a speed between 10 rpm and 1000 rpm, such as 100±10 rpm, for at least 5 minutes, thereby washing cleaned raw material 65 within vessel 602. Purified water 703 is decanted from mixing device 603 and replaced with fresh purified water 703. This process is repeated at least eleven additional times, thereby washing cleaned raw material 65 within vessel 602 at least twelve times.

In STEP 12260, cleaned raw material 65 is treated with disinfecting solution 705. In some embodiments, disinfecting solution 705 is brought in chamber 601 to a temperature of between 2° C. and 37° C., such as approximately 20° C. Cleaned raw material 65 and disinfecting solution 705 can comprise a ratio between 1:20 and 1:50, such as 1:30. Disinfecting solution 705 is added to mixing device 603. In some embodiments, at least 3000 mL of disinfecting solution 705 is added to mixing device 603. Mixing device 603 is stored in chamber 601 at a temperature of between 2° C. and 37° C., such as approximately 20° C. Mixing device 603 is combined with heating device 604 configured to agitate disinfecting solution 705 at a speed between 10 rpm and 1000 rpm, such as 100±10 rpm, for between 30 minutes and 240 minutes, such as 120±5 minutes, thereby washing cleaned raw material 65 within vessel 602. Disinfecting solution 705 is decanted from mixing device 603.

In STEP 12270, cleaned raw material 65 is washed with buffer solution 701. In some embodiments, buffer solution 701 is brought in chamber 601 to a temperature of between 2° C. and 37° C., such as approximately 20° C. Raw material 65 and buffer solution 701 can comprise a ratio between 1:20 and 1:50, such as 1:30. Buffer solution 701 is added to mixing device 603. In some embodiments, at least 3000 mL of buffer solution 701 is added to mixing device 603. Mixing device 603 is stored in chamber 601 at a temperature of between 2° C. and 37° C., such as approximately 20° C. Mixing device 603 is combined with heating device 604 configured to agitate buffer solution 701 at speed between 10 rpm and 1000 rpm, such as 100±10 rpm, for between 5 minutes and 60 minutes, such as 15±5 minutes, thereby washing cleaned raw material 65 within vessel 602. Buffer solution 701 is decanted from mixing device 603.

In STEP 12280, cleaned raw material 65 is washed with sterile water 708. In some embodiments, sterile water 708 is brought in chamber 601 to a temperature of between 2° C. and 37° C., such as approximately 20° C. Cleaned raw material 65 is washed with sterile water 708 at least two times. Cleaned raw material 65 and sterile water 708 can comprise a ratio between 1:20 and 1:50, such as 1:30. Sterile water 708 is added to mixing device 603. In some embodiments, at least 3000 mL of sterile water 708 is added to mixing device 603. Mixing device 603 is stored in chamber 601 at a temperature of between 2° C. and 37° C., such as approximately 20° C. Mixing device 603 is combined with heating device 604 configured to agitate sterile water solution at a speed between 10 rpm and 1000 rpm, such as 100±10 rpm, for between 5 minutes and 60 minutes, such as 15±5 minutes, thereby washing cleaned raw material 65 within vessel 602. Sterile water 708 is decanted from mixing device 603 and replaced with fresh sterile water solution. Mixing device 603 is combined again with heating device 604 configured to agitate sterile water 708 at a speed between 10 rpm and 1000 rpm, such as 100±10 rpm, for between 5 minutes and 60 minutes, such as 15±5 minutes, thereby washing cleaned raw material 65 within vessel 602 a second time. Sterile water 708 is decanted from mixing device 603.

In STEP 12290, cleaned raw material 65 is treated with one, two, or more excipients 711. In some embodiments, treatment with excipient 711 comprises sustained immersion in a solution of the excipient for time period that is sufficient to allow for its diffusion into raw material 65 until at least a volume (e.g. 25%, 50%, or 100%) of raw material 65 has been reached. In some embodiments, treatment with excipient 711 comprises application of the excipient to the surface of raw material 65 via a coating, dipping, and/or spraying process.

Upon the conclusion of Method 12200, cleaned raw material 65 comprises a decellularized segmented nerve tissue (referred to as “nerve segment” herein).

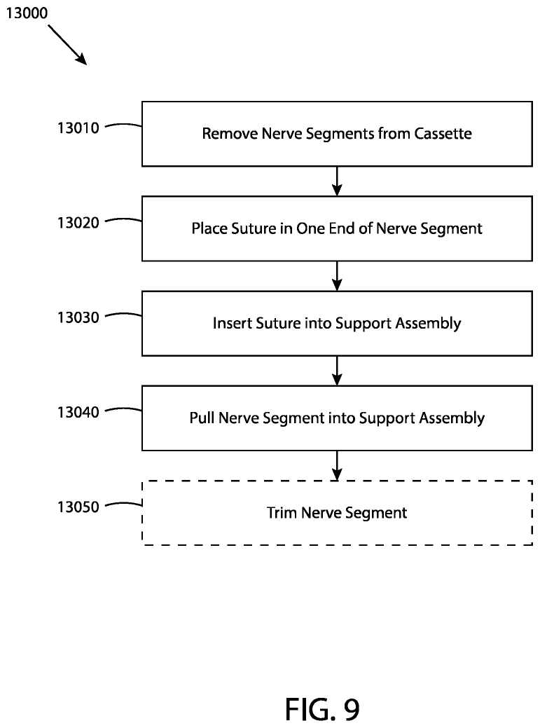

Referring now to FIG. 9, a method for inserting a nerve segment into a support assembly is illustrated, consistent with the present inventive concepts. Method 13000 can be configured to insert a nerve segment produced in Method 12000 described herein in reference to FIG. 8.

In STEP 13010, the nerve segment is removed from cassette 620.

In STEP 13020, a suture ligation is created and secured through a first end of the nerve segment. In some embodiments, a silk suture with a noose is created and secured through the first end of the nerve segment.

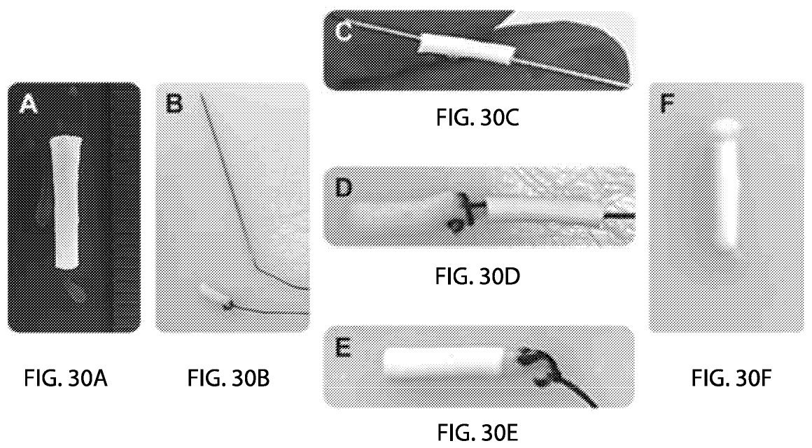

In STEP 13030, the suture is inserted into lumen 305 of support assembly 300 via proximal end 304, as described herein in reference to FIG. 30.

In STEP 13040, the suture is pulled through support assembly 300 such that the nerve segment is advanced into lumen 305. In some embodiments, the suture is pulled through support assembly 300 until the first end of the nerve segment is proximate distal end 306 of support assembly 300.

In STEP 13050, comprising an optional step, any portion of the nerve segment that is not contained within support assembly 300 can be removed (e.g. cut, or otherwise detached) and discarded. In some embodiments, support assembly 300 comprises a length of approximately 1 cm and any portion of the nerve segment that extends beyond support assembly 300 is removed or otherwise detached (e.g. the nerve segment is cut to comprise a length of approximately 1 cm).

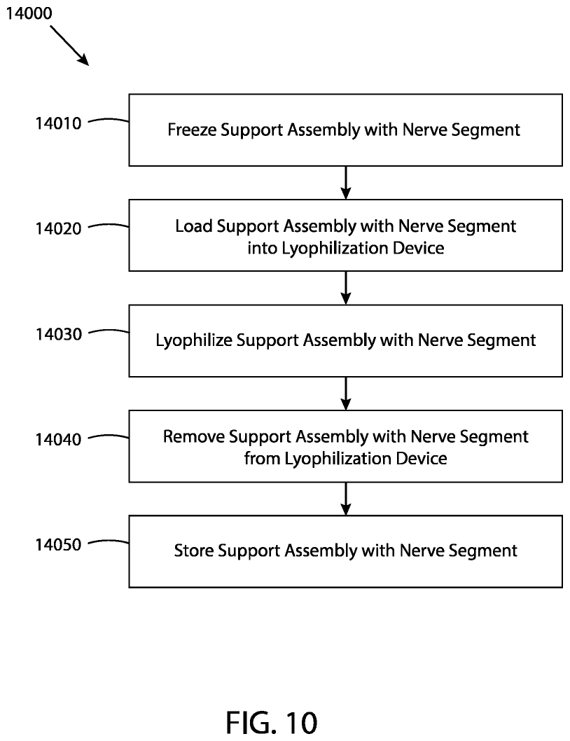

Referring now to FIG. 10, a method for lyophilizing a nerve segment contained within a support assembly is illustrated, consistent with the present inventive concepts. Method 14000 can be configured to lyophilize a nerve segment contained within a support assembly produced in Method 13000 described herein in reference to FIG. 9.

In STEP 14010, support assemblies 300 comprising nerve segments can be transferred to, and stored within, chamber 601 comprising heat flow controls and/or methods configured to freeze the nerve segments at a defined rate of freezing and with a defined directionality of the freezing front. Such rate and directionality of freezing can be intended to affect the size and orientation of the water crystals within the nerve segments. The water crystal geometries can be configured to affect the geometry of induced macroporosity, pore size, pore orientation, and tissue permeability resulting after lyophilization. In some embodiments, the nerve segment can be placed into a thermally controlling system placed inside chamber 601. Such thermally controlling system can comprise: thermally insulating materials; selective openings to the external environment into chamber 601; internal electrical resistances connected to a controller to provide heating patterns within the geometry of nerve segment; channels for the flow of warmed fluid heated by a heat exchanger connected to a controller and also able to provide heating patterns within nerve segment; and combinations of these. Such thermally controlling system can be programmed to control the directionality and speed of the freezing front for the nerve segments. Such thermally controlling system can create, for example, a longitudinally oriented freezing front able to move at a selected speed throughout the main axis of the nerve segment to induce the growth of the water crystal along a main axis. In some embodiments, support assemblies 300 comprising nerve segments are stored in chamber 601 overnight.

In STEP 14020, one, two, or more support assemblies 300 comprising nerve segments are loaded into lyophilization device 606. In some embodiments, support assemblies 300 comprising nerve segments are loaded into a preconditioned lyophilization device 606.

In STEP 14030, support assemblies 300 comprising nerve segments are lyophilized via lyophilization device 606. In some embodiments, support assemblies 300 comprising nerve segments are lyophilized for 72 hours.

In STEP 14040, support assemblies 300 comprising nerve segments are removed from lyophilization device 606.

In STEP 14050, support assemblies 300 comprising nerve segments are transferred to, and stored within, chamber 601 comprising a temperature between 2° C. and 8° C.

Upon the conclusion of Method 14000, the nerve segments comprise lyophilized nerve segments (referred to as “lyophilized nerve segment” herein).

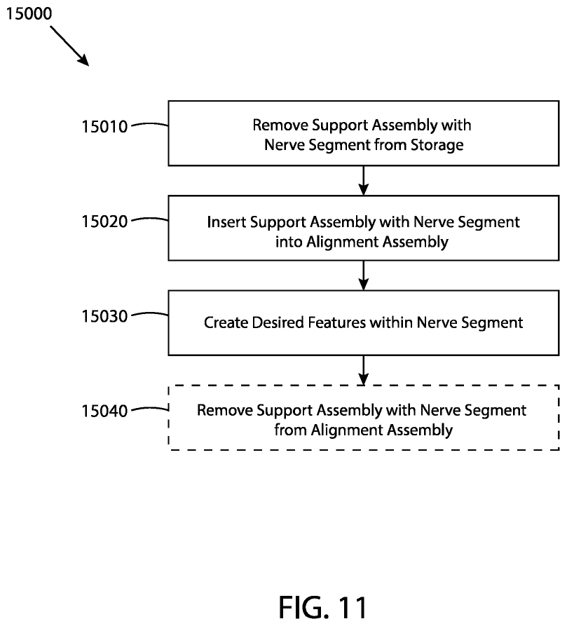

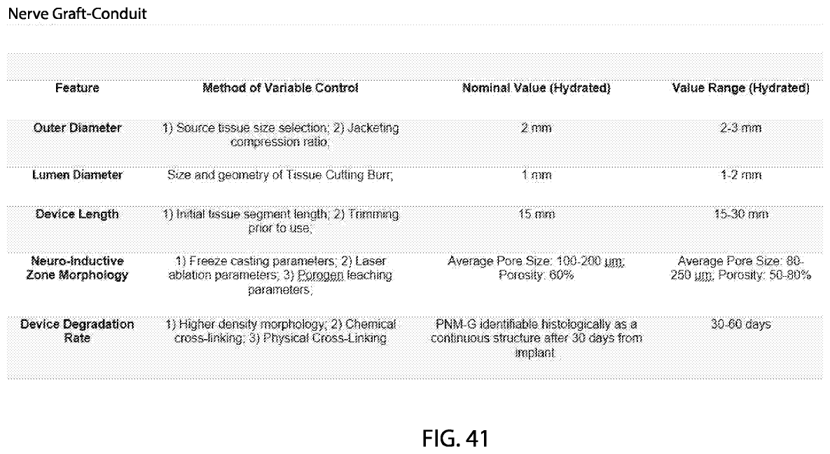

Referring now to FIG. 11, a method for creating desired features within a nerve segment is illustrated, consistent with the present inventive concepts. Method 15000 can be configured to create one, two, or more features (e.g. lumen 2122, socket 2210, anchoring tab 2241, etc.) through lyophilized nerve segments produced in Method 14000 described herein in reference to FIG. 10. In some embodiments, alternate methods, such as laser ablation, electrosurgery, or ultrasound cutting, are utilized to create the one, two or more features, such as described in reference to FIG. 41. In some embodiments, Method 15000 can be configured to increase and/or decrease permeability of the nerve segment radially, longitudinally, circumferentially, or combinations of these.

In STEP 15010, support assemblies 300 comprising lyophilized nerve segments are removed from chamber 601.

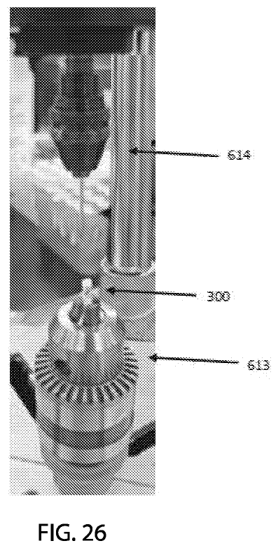

In STEP 15020, one of proximal end 304 and distal end 306 of support assembly 300 comprising the lyophilized nerve segment is inserted and secured within alignment assembly 613, as described hereinbelow in reference to FIG. 26. Alignment assembly 613 can be positioned below pin assembly 614 comprising a pin therein.

In STEP 15030, one, two, or more features (e.g. lumen 2122, socket 2210, anchoring tab 2241, etc.) are created within the lyophilized nerve segment.

In some embodiments, pin assembly 614 is lowered towards alignment assembly 613 comprising support assembly 300 such that the pin penetrates at least a portion of the lyophilized nerve segment. The pin can be configured to create a channel extending through at least a portion of the lyophilized nerve segment. In some embodiments, the pin creates a channel that extends the full length of the lyophilized nerve. Pin assembly 614 can be raised away from alignment assembly 613 such that the pin is removed from the lyophilized nerve segment. Pin assembly 614 can be repositioned above alignment assembly 613 comprising support assembly 300. In some embodiments, alignment assembly 613 can be used to reposition support assembly 300 below pin assembly 614.

In STEP 15040, comprising an optional step, support assembly 300 comprising the lyophilized nerve segment is released and removed from alignment assembly 613. In some embodiments, support assembly 300 includes one or more components that can be inserted into the channel(s) created to maintain the channel(s) during rehydration of the lyophilized nerve segment.

Upon the conclusion of Method 15000, the lyophilized nerve segment comprises a nerve graft-conduit (referred to as “PNM-G 2100” herein) or nerve cap-graft (referred to as “PNM-CAP 2200” herein).

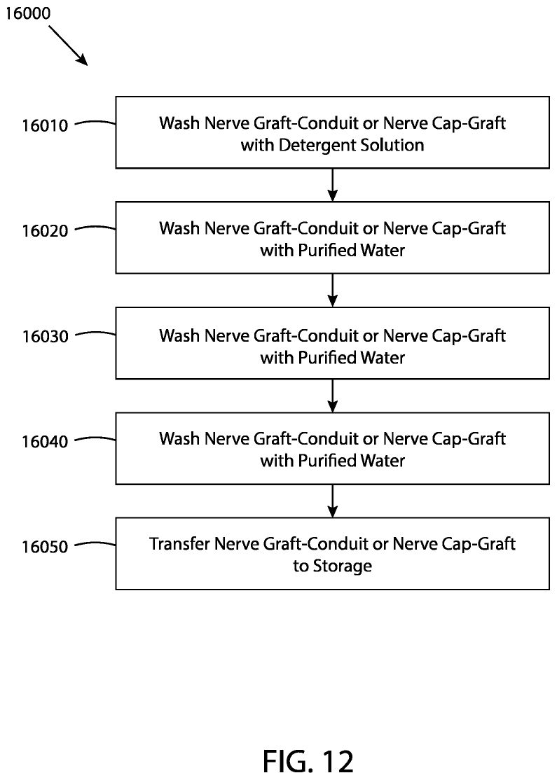

Referring now to FIG. 12, a method for decellularizing a nerve graft-conduit or nerve cap-graft is illustrated, consistent with the present inventive concepts. Method 16000 can be configured to decellularize PNM-G 2100 or PNM-CAP 2200 prepared in Method 15000 described herein in reference to FIG. 11.

In STEP 16010, PNM-G 2100 or PNM-CAP 2200 is treated (e.g. washed) with detergent solution 706. In some embodiments, PNM-G 2100 or PNM-CAP 2200 is treated with detergent solution 706 for a duration of approximately 8 hours.

In STEP 16020, PNM-G 2100 or PNM-CAP 2200 is washed with purified water 703. In some embodiments, PNM-G 2100 or PNM-CAP 2200 is washed with purified water 703 for a duration of at least 5 minutes at least six times.

In STEP 16030, PNM-G 2100 or PNM-CAP 2200 is washed with purified water 703. In some embodiments, PNM-G 2100 or PNM-CAP 2200 is washed with purified water 703 for a duration of at least 24 hours.

In STEP 16040, PNM-G 2100 or PNM-CAP 2200 is washed with purified water 703. In some embodiments, PNM-G 2100 or PNM-CAP 2200 is washed with purified water 703 for a duration of at least 5 minutes at least six times.

In STEP 16040, PNM-G 2100 or PNM-CAP 2200 is transferred to, and stored within, chamber 601.

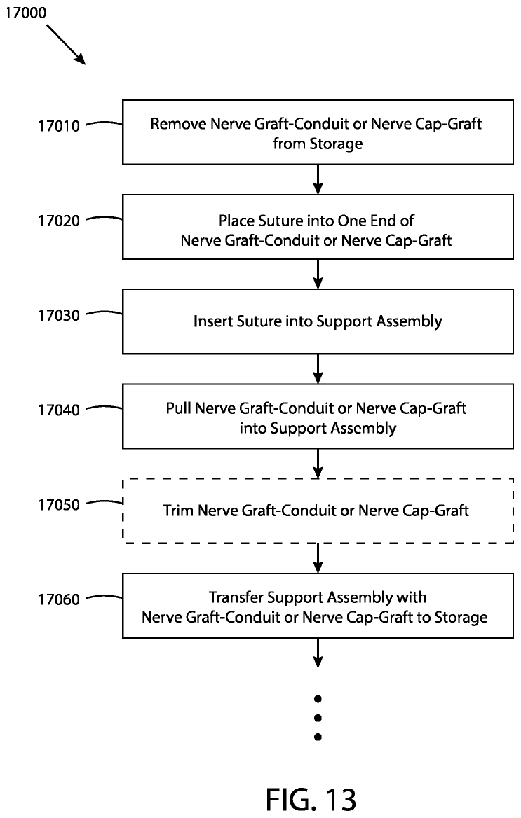

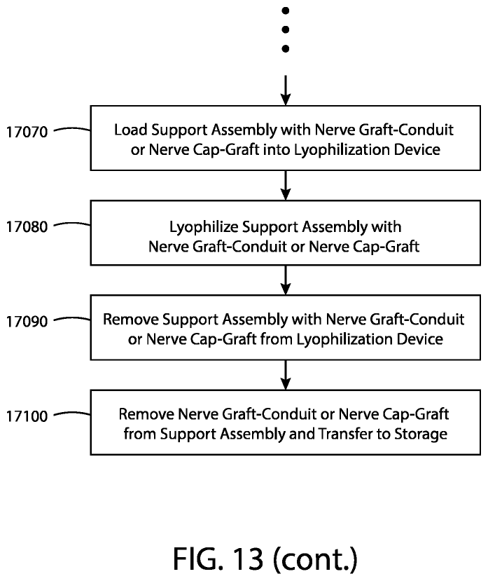

Referring now to FIG. 13, a method for stabilizing a nerve graft-conduit or nerve cap-graft is illustrated, consistent with the present inventive concepts. Method 17000 can be configured to stabilize PNM-G 2100 or PNM-CAP 2200 prepared in Method 16000 described herein in reference to FIG. 12.

In STEP 17010, PNM-G 2100 or PNM-CAP 2200 is removed from chamber 601.

In STEP 17020, a suture ligation is created and secured through a first end of the PNM-G 2100. In some embodiments, a silk suture with a noose is created and secured through the first end of PNM-G 2100 or PNM-CAP 2200.

In STEP 17030, the suture is inserted into lumen 305 of support assembly 300 via proximal end 304, as described herein in reference to FIG. 30.

In STEP 17040, the suture is pulled through support assembly 300 such that PNM-G 2100 is advanced into lumen 305. In some embodiments, the suture is pulled through support assembly 300 until the first end of PNM-G 2100 or PNM-CAP 2200 is proximate distal end 306 of support assembly 300.

In STEP 17050, comprising an optional step, any portion of PNM-G 2100 or PNM-CAP 2200 that is not contained within support assembly 300 can be removed (e.g. cut, or otherwise detached) and discarded. In some embodiments, support assembly 300 comprises a length of approximately 1 cm and any portion of PNM-G 2100 or PNM-CAP 2200 that extends beyond support assembly 300 is removed or otherwise detached (e.g. PNM-G 2100 or PNM-CAP 2200 is cut to comprise a length of approximately 1 cm).

In STEP 17060, support assemblies 300 comprising PNM-G 2100 or PNM-CAP 2200 can be transferred to, and stored within, chamber 601 comprising a temperature of −80° C. In some embodiments, support assemblies 300 comprising PNM-G 2100 or PNM-CAP 2200 are stored in chamber 601 for a few hours.

In STEP 17070, one, two, or more support assemblies 300 comprising PNM-G 2100 or PNM-CAP 2200 are loaded into lyophilization device 606. In some embodiments, support assemblies 300 comprising PNM-G 2100 or PNM-CAP 2200 are loaded into a preconditioned lyophilization device 606.

In STEP 17080, support assemblies 300 comprising PNM-G 2100 or PNM-CAP 2200 are lyophilized via lyophilization device 606. In some embodiments, support assemblies 300 comprising PNM-G 2100 or PNM-CAP 2200 are lyophilized for 72 hours.

In STEP 17090, the lyophilized PNM-G 2100 or PNM-CAP 2200 are removed from lyophilization device 606.

In STEP 17100, PNM-G 2100 or PNM-CAP 2200 are removed from support assembly 300, and stored at a temperature of between 15° C. and 25° C. in a very dry and sterile environment prior to packaging.

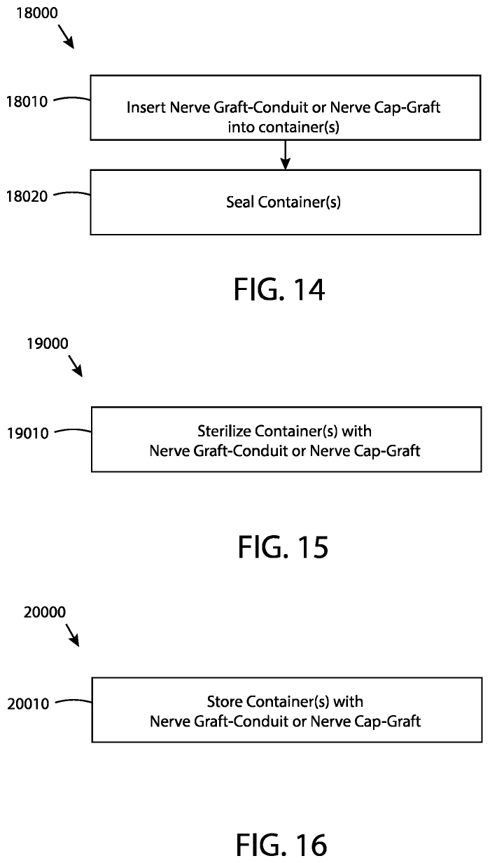

Referring now to FIG. 14, a method for packaging a nerve graft-conduit or nerve cap-graft is illustrated, consistent with the present inventive concepts. Method 18000 can be configured to package PNM-G 2100 or PNM-CAP 2200 produced in Method 17000 described herein in reference to FIG. 13. PNM-G 2100 or PNM-CAP 2200 can be packaged for bulk storage and/or sterilization. Method 18000 is configured to be performed within an environment suitable for aseptic processing, such that that materials, devices, and components utilized in Method 18000 are transferred to and/or contained within an environment suitable for aseptic processing. For example, Method 18000 is performed utilizing a sterile work area and/or sterile handling, such as to prevent or otherwise reduce contamination from microorganisms (e.g. bacteria, fungi, virus, etc.).

In STEP 18010, insert PNM-G 2100 or PNM-CAP 2200 into one or more containers.

In STEP 18020, seal the containers.

Referring now to FIG. 15, a method for an irradiation based sterilization of a container comprising a nerve graft-conduit or nerve cap-graft is illustrated, consistent with the present inventive concepts. Method 19000 can be configured to sterilize the packaged PNM-G 2100 or PNM-CAP 2200 produced in Method 18000 described herein in reference to FIG. 14.

In STEP 19010, the containers comprising PNM-G 2100 or PNM-CAP 2200 are sterilized.

The containers comprising PNM-G 2100 or PNM-CAP 2200 can be sterilized via gamma irradiation, such that the containers are exposed to gamma radiation (e.g. Cobalt 60).

The containers comprising PNM-G 2100 or PNM-CAP 2200 can be sterilized via electron-beam irradiation (“e-beam irradiation” herein), such that the containers are exposed to a stream of electrons.

Referring now to FIG. 16, a method for shipping and/or storing a container comprising a nerve graft-conduit or nerve cap-graft is illustrated, consistent with the present inventive concepts. Method 20000 can be configured to ship and/or store the sterilized PNM-G 2100 or PNM-CAP 2200 produced in Method 19000 described herein in reference to FIG. 15.

In STEP 20010, the containers comprising PNM-G 2100 or PNM-CAP 2200 are stored.

The containers comprising PNM-G 2100 or PNM-CAP 2200 can be stored at a temperature of between 15° C. and 25° C., such as at a temperature of approximately 20° C.

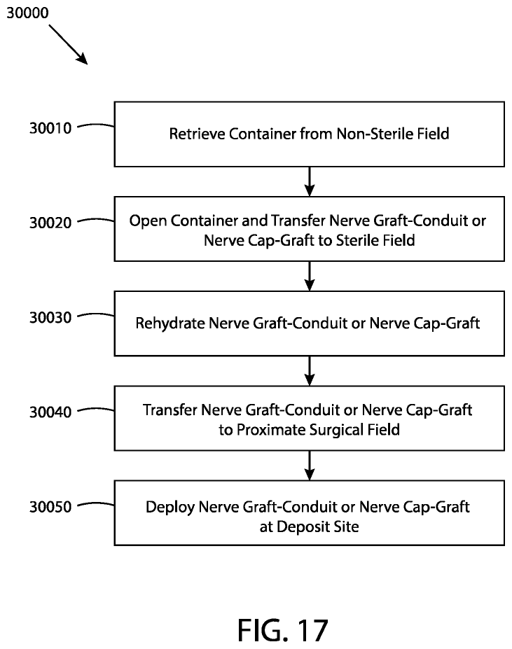

Referring now to FIG. 17, a method for deploying a device comprising a nerve graft-conduit or nerve cap-graft is illustrated, consistent with the present inventive concepts. Method 30000 can be configured to deploy a nerve graft-conduit comprising PNM-G 2100 or never cap-graft PNM-CAP 2200 as described herein.

As described herein in reference to Method 30000, and for non-limiting proposes, the deposit site comprises one, two, or more injured nerves.

In STEP 30010, the container comprising PNM-G 2100 or PNM-CAP 2200 is retrieved from a non-sterile field (e.g. storage) by a first, non-sterile operator (e.g. circulating nurse, etc.) and is transported to the room within which the clinical procedure is to be performed (e.g. treatment room, operating room).

In STEP 30020, the first operator opens the container in proximity to the sterile field where a second sterile operator (e.g. scrub nurse, surgeon, etc.) assists with the transfer of all internal components (e.g. PNM-G 2100 or PNM-CAP 2200) to the sterile field using aseptic techniques.

In STEP 30030, the second operator rehydrates PNM-G 2100 or PNM-CAP 2200. In some embodiments, PNM-G 2100 or PNM-CAP 2200 is immersed in 1×PBS for a duration of between 1 minute and 10 minutes. Upon rehydration, PNM-G 2100 or PNM-CAP 2200 is configured to increase in size logarithmically.

In STEP 30040, the second operator transfers the rehydrated PNM-G 2100 or PNM-CAP 2200 to a third, sterile operator (e.g. physician, surgeon, etc.) proximate to a surgical field.

In STEP 30050, the third operator deploys, or otherwise delivers, PNM-G 2100 or PNM-CAP 2200 at the deposit site. In some embodiments, the third operator alters the size of PNM-G 2100 or PNM-CAP 2200 to a desired length prior to deploying PNM-G 2100 or PNM-CAP 2200 at the deposit site, such as by trimming or otherwise reducing the length of PNM-G 2100 or PNM-CAP 2200.

In some embodiments, and as shown in FIG. 4A, the deposit site comprises a nerve gap and PNM-G 2100 is configured to connect two nerve stumps. The third operator can create two proximal and two distal sutures (e.g. 9-0 nylon monofilament suture) to secure PNM-G 2100 to the nerve stumps.

In some embodiments, and as shown in FIG. 4B, the deposit site comprises an amputated nerve and PNM-G 2100 or PNM-CAP 2200 is attached to an end portion amputated nerve. The third operator can create two proximal sutures (e.g. 9-0 nylon monofilament suture) to secure PNM-G 2100 or PNM-CAP 2200 to the amputated nerve.

In some embodiments, and as shown in FIG. 4C, the deposit site comprises a nerve transfer and PNM-G 2100 is configured to connect a healthy nerve to an injured nerve. The third operator can create two proximal and two distal sutures (e.g. 9-0 nylon monofilament suture) to secure PNM-G 2100 to the healthy nerve and the injured nerve.

Referring now to FIG. 18, a perspective view of a sizing tool is illustrated, consistent with the present inventive concepts. Sizing tool 90 can be constructed and arranged to measure and/or alter the one or more dimensions (e.g. length, diameter, etc.) of PNM-G 2100 or PNM-CAP 2200 and/or one or more dimensions (e.g. length, diameter, etc.) of the lumen 2122, socket 2210, and/or anchoring tab 2214 prior to implantation.

Sizing tool 90 can comprise elements with which the diameter of PNM-G 2100 or PNM-CAP 2200 and/or the diameter of the deposit site comprising a nerve stump is measured. In some embodiments, sizing tool 90 comprises two, three, or more circular openings, holes 92a-c as shown. Each hole 92 can comprise a unique outer diameter OD. For example, hole 92a can comprise an outer diameter smaller than hole 92b, which comprises an outer diameter smaller than hole 92c. An operator (e.g. physician, surgeon, etc.) can insert PNM-G 2100 or PNM-CAP 2200 through each hole 92 to measure or otherwise confirm the device's diameter. In some embodiments, the operator can trim or otherwise alter the diameter of PNM-G 2100 or PNM-CAP 2200 to achieve a desired diameter prior to implantation. An operator (e.g. physician, surgeon, etc.) can insert proximal and/or distal nerve stumps through each hole 92 to measure or otherwise confirm the nerve diameter to be matched by device 100.

Sizing tool 90 can further comprise elements with which the length of PNM-G 2100 or PNM-CAP 2200 is measured. In some embodiments, sizing tool 90 comprises two, three, or more protrusions, protrusions 94a-d as shown. Each protrusion 94 can comprise a unique width W. For example, protrusion 94a comprises a width smaller than protrusion 94b, which comprises a width smaller than protrusion 94c, which comprises a width smaller than protrusion 94d. An operator (e.g. physician, surgeon, etc.) can compare the width of each protrusion 94 to the distance between two nerve stumps to measure or otherwise confirm the desired length of PNM-G 2100 or PNM-CAP 2200. In some embodiments, the operator can trim or otherwise alter the length of PNM-G 2100 or PNM-CAP 2200 to achieve the desired length prior to implantation.

Sizing tool 90 can further comprise one, two, or more elements with which the diameter of the lumen 2122 of PNM-G 2100 or socket 2214 of PNM-CAP 2200 is measured. In some embodiments, sizing tool 90 comprises two, three, or more cylindrical protrusions, protrusions 94a-d as shown. Each protrusion 94 can comprise a unique outer diameter OD. For example, protrusion 94a can comprise a diameter that is smaller than the diameter of protrusion 94b, which comprises a diameter that is smaller than the diameter of protrusion 94c, which comprises a diameter that is smaller than the diameter of protrusion 94d. An operator (e.g. physician, surgeon, etc.) can insert each protrusion 94 into at least a portion of lumen 2122 of PNM-G 2100 or socket 2214 of PNM-CAP 2200 to measure or otherwise confirm the diameter of lumen 2122 or the diameter of socket 2214 after rehydration. Sizing tool 90 can further comprise a length measurement element, ruler 96, comprising two or more markings at regular intervals configured to measure a distance between two reference points. In some embodiments, ruler 96 is utilized to measure a length of PNM-G 2100 or PNM-CAP 2200.

Referring now to FIG. 19, a graphical representation of the rehydration of a nerve graft-conduit or nerve cap-graft is illustrated, consistent with the present inventive concepts. The nerve graft-conduit can comprise PNM-G 2100 and the nerve cap-graft can comprise PNM-CAP 2200 as described herein. During rehydration, the compressed, lyophilized PNM-G 2100 or PNM-CAP 2200 increases in size logarithmically, as shown. In some embodiments, PNM-G 2100 or PNM-CAP 2200 is rehydrated via immersion in 1×PBS for a duration of between 1 minute and 10 minutes.

Referring now to FIGS. 20A and B, photographs of unprocessed and processed nerve tissue are illustrated, respectively, consistent with the present inventive concepts. Unprocessed nerve tissue comprises excess connective tissue, as shown in FIG. 20A, that is manually, mechanically, chemically, and/or physically removed to result in processed nerve tissue, as shown in FIG. 20B.

Referring now to FIGS. 21A and B, photographs of unprocessed and processed nerve tissue are illustrated, respectively, consistent with the present inventive concepts. Unprocessed nerve tissue comprises excess connective tissue, as shown in FIG. 21A, that is manually, mechanically, chemically, and/or physically removed to result in processed nerve tissue, as shown in FIG. 21B.

Referring now to FIGS. 22A and B, photographs of an untrimmed nerve branch system and trimmed nerve segments are illustrated, respectively, consistent with the present inventive concepts. Untrimmed nerve segments comprise a native nerve branch system, as shown in FIG. 22A. The native nerve branch system is trimmed or otherwise cut into two or more nerve segments according to one, two, or more features (e.g. nerve size, location, fascicular complexity, etc.), such as shown in FIG. 22B. The nerve segments can be trimmed or otherwise cut to comprise various diameters and lengths.

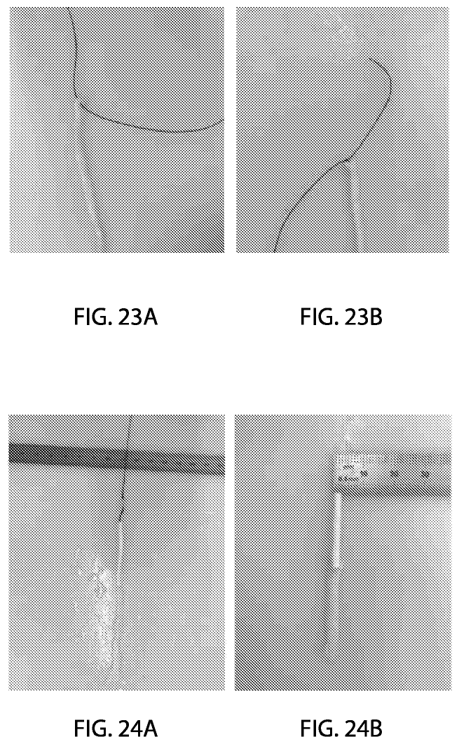

Referring now to FIGS. 23A and B, photographs of a nerve segment prepared for insertion into a nerve jacket are illustrated, consistent with the present inventive concepts. The nerve jacket can be constructed and arranged similar to that of support assembly 300 described herein. A single suture is created and secured to a first end of the nerve segment. In some embodiments, the suture comprises a silk suture comprising a noose configured to choke the first end of the nerve segment.

Referring now to FIGS. 24A and B, photographs of a nerve segment inserted into a nerve jacket are illustrated, consistent with the present inventive concepts. The nerve jacket can be constructed and arranged similar to that of support assembly 300 described herein. The suture created in FIGS. 23A and B can be threaded through lumen 305 of support assembly 300, such that the nerve segment is pulled and compressed within lumen 305. In some embodiments, support assembly 300 comprising the nerve segment therein is trimmed or otherwise cut to a length of 1 cm.

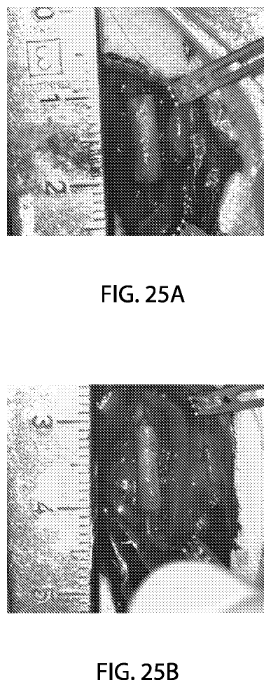

Referring now to FIGS. 25A and B, photographs of a nerve graft-conduit implanted at a nerve injury site are illustrated, consistent with the present inventive concepts. Following rehydration as described herein, PNM-G 2100 can be placed proximate a nerve injury site, such as between two nerve stumps. At least two proximal and at least two distal sutures can be created, thereby securing the interface between PNM-G 2100 with each nerve stump

Referring now to FIG. 26, a photograph of an alignment assembly positioned below a pin assembly is illustrated, consistent with the present inventive concepts. Alignment assembly 613 can comprise a Dremel keyless chuck configured to secure support assembly 300 comprising a nerve segment, as shown. Alignment assembly 613 can be positioned directly below pin assembly 614. Pin assembly 614 can be lowered towards alignment assembly 613 to create one, two, or more channels within the nerve segment, as described hereinabove in reference to FIG. 11. Alignment assembly 613 can also comprise a three-dimensional linear stage that allows movement of the support assembly 300 in X, Y, Z directions. Alignment assembly 613 can be raised towards pin assembly 614 using the linear stage to create one, two, or more channels within the nerve segment.

Referring now to FIG. 27, a schematic view of cassette for securing one or more nerve segments is illustrated, consistent with the present inventive concepts. Nerve cassette 620 can comprise a housing 621 comprising one, two, or more channels 625 each configured to receive a nerve segment, such as raw material 65 described herein. Housing 621 can further comprise one or more dividers 623 configured to provide a barrier between adjacent channels 625. Housing 621 can comprise one or more fluid-permeable materials configured to allow for the passage of a fluid between cassette 620 (e.g. channels 625 therein) and an external environment.

Channels 625 can be configured to prevent or otherwise reduce bunching of the nerve segments. Each channel 625 can comprise a straight and/or spiral shape.

Once inserted into a channel 625, each nerve segment can be removably attached to housing 621 via one or more anchoring elements 627. In some embodiments, both ends of the nerve segment are secured via an anchoring element 627.

Referring now to FIG. 28, a perspective view of a support assembly for manufacturing a nerve graft-conduit is illustrated, consistent with the present inventive concepts. Support assembly 300 can comprise a housing 301 constructed and arranged as a cylindrical tube with a lumen 305 therethrough. Housing 301 comprises a proximal end 304 and a distal end 306. Support assembly 300 can be configured to receive raw material 65, ECM 120, and/or PNM-G 2100 (collectively “contents” herein).

Support assembly 300 can comprise a plurality of pores 309. Each pore 309 can have diameter of between 0.1 micron and 500 microns. Pores 309 can be distributed uniformly along the longitudinal and/or circumferential directions of housing 301. In some embodiments, pores 309 are configured to allow for the passage of a fluid or vapor between support assembly 300 (e.g. contents therein) and an external environment. In some embodiments, a vacuum source is applied to support assembly 300 and pores 309 are configured to apply a uniform vacuum to the contents within support assembly 300. The vacuum source can be configured to maintain uniform contact between an outer surface of the contents and an inner surface of support assembly 300. One or more thermal and/or physical treatments can be applied concurrently with the vacuum source, such as to induce the formation of coaxial layers within the contents as a result of tissue contraction and/or shearing. Support assembly 300 can comprise an insulating material to prevent the passage of a fluid, vapor, and/or heat between the tissue contained within support assembly 300 and an external environment (except for in a select axis).

Support assembly 300 can comprise a material selected from the group consisting of: expanded and non-expanded poly fluoro tetraethylene; polysulfones; cellulose acetate; polyamide; polyvinylidene fluoride; polysulfone; polyethersulfone; polyvinyl chloride; polyimide; polyacrylonitrile; polyethylene glycol; polyvinyl alcohol; poly(methacrylic acid); poly(arylene ether ketone); poly(ether imide); and polyaniline nanoparticles; stainless steel, such as 316L stainless steel; aluminum; cobalt alloy; titanium alloy; and combinations of these. In some embodiments, support assembly 300 comprises a hydrophobic, expanded poly fluoro tetraethylene (ePTFE) tube comprising a porosity of between 50% and 60%, a wall thickness of between 0.3 mm and 0.8 mm, and/or a length of between 1 mm and 10 mm.

Support assembly 300 can further comprise one, two, or more elements configured to impart structural modifications to the contents therein. For example, support assembly 300 can be configured to impart a plurality of channels (e.g. lumens 2122) to raw material 65, ECM 120, and/or PNM-G 2100 via ablation and/or sublimation processes using an excimer laser.

Support assembly 300 can be constructed and arranged to slidingly receive contents therein, such as described hereinabove in reference to FIGS. 23A,B and FIGS. 24A,B. For example, a nerve segment can be pulled into assembly 300 (e.g. lumen 305) using a suture ligation anchored at one end of the nerve segment. The suture ligation can be pulled through assembly 300 (e.g. lumen 305) until a desired portion of the nerve segment is positioned within the assembly.

Support assembly 300 can be constructed and arranged as a compressive, porous, tubular structure configured to constrict or restrict the contents therein. Support assembly 300 can be configured to promote the homogenous and/or unidirectional lyophilization of the contents therein. Support assembly 300 can be configured to homogenize the diameter of the contents therein. Support assembly 300 can be configured to reduce the size of the contents therein according to a diameter conversion factor. Support assembly 300 can be configured to alter the length of the contents therein according to a conversion factor. In some embodiments, support assembly 300 further includes a component to be inserted into a socket or lumen of the contents therein. This component can maintain (e.g. reinforce) the socket or lumen during rehydration, disinfection, and/or storage of the object (e.g. implant 20, ECM 120, PNM-G 2100, PNM-CAP 2200).

Support assembly 300 and the related methods can be configured to manufacture at least a portion of implant 20, ECM 120, PNM-G 2100, and/or PNM-CAP 2200 as described herein.

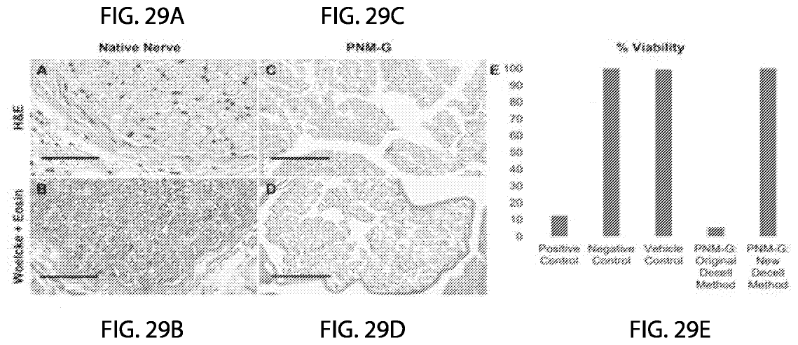

Referring to FIGS. 29-44, applicant has developed a nerve graft-conduit for nerve gap injury repair (PNM-G 2100, herein) and a neuro-inhibitory nerve cap-graft (PNM-CAP 2200 herein), which is manufactured using methodologies equivalent to those described for PNM-G 2100, but with some modifications.

Applicant has developed PNM-G 2100 to address the need for solutions for nerve gap repair, which requires a physical bridge between nerve endings. The resulting PNM-G 2100 is a graft-conduit device, which applicant has demonstrated to be fully decellularized, biocompatible, and non-cytotoxic, and with shape, size, and surgical handling characteristics suitable for nerve gap repair applications. Upon PNM-G 2100 implantation in a rodent nerve gap injury model, applicant observed that PNM-G 2100 functioned as a potent and effective inhibitor, rather than inducer of nerve growth. This unexpected outcome is highly desired, yet seldom achieved with current approaches for the prevention of painful neuroma. Applicant has determined that the neuro-inhibitory activity of PNM-G 2100 resulted from both the collapse of the device lumen and the excessive compression of the annular matrix surrounding it.

Additionally, applicant has developed a neuro-inhibitory nerve cap-graft (PNM-CAP 2200) configured to the formation of neuroma following nerve damage sustained during limb amputations. PNM-CAP 2200 comprises a decellularized nerve graft-conduit similar to PNM-G 2100, but with the distal end of the material either compressed to form an anchoring tab 2214 or ligated to compress the matrix at the distal end to create a progressive restriction of nerve growth. Applicant's findings demonstrate that PNM-CAP 2200 has potential for use in the field of neuroma prevention and/or treatment.

As described hereinbelow, applicant has performed a wide range of product development activities as well as in vitro and in vivo testing to address two objectives: develop an off-the-shelf, biodegradable nerve graft-conduit with geometrical, mechanical, structural, and biological properties suitable to bridge nerve gap injuries and actively induce nerve regeneration; and assess the functionality of PNM-G 2100 for gap repair applications with and without ECM 120 as a luminal filler in a small animal model of nerve gap repair.

Within these studies, applicant has developed methods for effective decellularization of nerve graft-conduits, methods for controlling graft-conduit size and shape, methods for compressing and lyophilizing nerve segments, and methods for graft-conduit lumen creation. Overall, the compression of the peri-luminal matrix and collapsible nature of the lumen of the graft-conduit led to inhibition rather than induction of functional regeneration following nerve injury and repair. In particular, the resulting grafts functioned as an effective substrate for controlling unregulated neurite outgrowth, and inhibited neuroma formation without adverse reactions to the implant when tested in rodent models.

Referring now to FIGS. 29A-E, histological images of native nerve tissue versus decellularized nerve tissue and assay results for the decellularized nerve tissue are illustrated, consistent with the present inventive concepts.

PNM-G 2100 comprises an off-the-shelf graft-conduit that is fully decellularized and non-cytotoxic, with shape, size, and handling characteristics suitable for surgical use. Applicant sought to ensure the device materials were fully decellularized and free of myelin, a nerve tissue lipoprotein that can cause a significant inflammatory response. Applicant began by testing the baseline decellularization protocol used for the peripheral nerve matrix (PNM), which is obtained from small, morselized, short segments of porcine sciatic nerve. The nerve segments were then exposed to a series of chemical and enzymatic treatments designed to remove cellular content while maintaining tissue structure and composition. Applicant discovered that larger nerve tissue segments used for PNM-G 2100 did not fully decellularize using the ECM 120 process. Furthermore, in vitro cytotoxicity assessments evidenced that the grafts were cytotoxic due to residual reagents from the decellularization process. This was found to be due to a decrease in the tissue surface area-to-volume ratio impacting mass transport for the larger/longer PNM-G 2100 tissue segments when compared to the morselized ones used for PNM.

For these reasons, applicant has studied the effects of each individual reagent used in the PNM-G 2100 decellularization process, and optimized the protocols with the goal of maximizing the removal of unwanted biological content (i.e. cell debris, DNA, and myelin) and cytotoxic residues.

Referring now to FIGS. 30A-F, a generalized nerve jacketing process is illustrated, consistent with the present inventive concepts. Human nerve repair applications require the use of grafts and conduits with a controllable range of diameters and lengths to address the spectrum of anatomical and surgical scenarios. Applicant has developed a process that utilizes microporous tubular ‘jackets’ to control the graft-conduit geometry by: restricting the outer diameter of the graft-conduit to the desired size; eliminating the natural taper/reduction in diameter of the nerve from proximal to distal ends; imparting a straight, aligned, and round cross-sectional geometry to the nerve graft-conduit; and creating a temporary external mechanical support for the nerve segment to support additional features, such as the creation of a lumen. Nerve jackets may also enable future features including preferential sublimation of water during lyophilization along the longitudinal direction of the decellularized nerve segment to create aligned micro-porosity, and partial sublimation along the radial direction of the nerve segment to improve tissue permeability. FIG. 30A shows a nerve segment following a first water bath. FIG. 30B shows the nerve segment ligated distally. FIG. 30C shows the free end of a snare that has been threaded through the nerve jacketed channel. FIG. 30D shows the nerve jacket slid along the snare thread to meet the nerve segment. FIG. 30E shows the ligated end of the nerve segment pulled through the nerve jacket. FIG. 30F shows the resulting jacketed nerve segment.

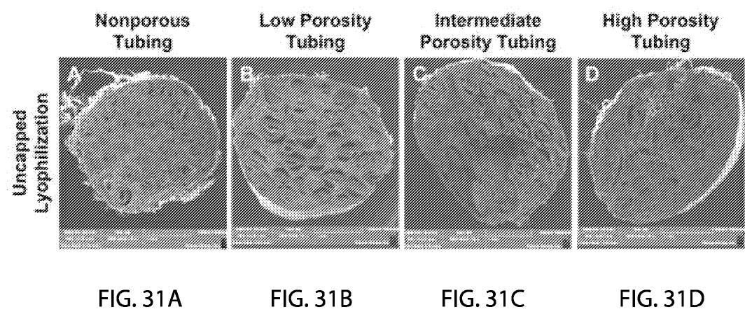

Referring now to FIGS. 31A-D, scanning electron microscopy (SEM) images for morphological assessment of nerve graft-conduits following lyophilization in tubing with various porosities are illustrated, consistent with the present inventive concepts. Applicant has conducted tests on four different nerve jackets; three comprising expanded polytetrafluoroethylene (ePTFE) tubing with varying porosities (e.g. low porosity of 56% as shown in FIG. 31B, intermediate porosity of 67% as shown in FIG. 31C, and high porosity of 79% as shown in FIG. 31D) and the other comprising nonporous silicon tubing as shown in FIG. 31A. Results demonstrate that ePTFE jacketed groups, when compared to the nonporous tubing group, have a more porous structure on examination under SEM. ePTFE jackets appear to improve matrix porosity compared to those made with nonporous nerve jackets. Therefore, applicant has tested PNM-G 2100 made with varying porous nerve jackets in a short-term pilot rat sciatic gap model. Results from the gap model demonstrate positive feedback regarding the texture, suturability, and handling characteristics of the rehydrated PNM-G 2100; particularly those formed using the lower porosity ePTFE jacket material. However, applicant observed a significant diameter mismatch between the nerve and PNM-G 2100. Upon further investigations, it was found that the initial pre-jacket nerve diameter was an important determinant of graft-conduit diameter following rehydration of PNM-G 2100. This prompted applicant to revise the source tissue diameter to jacket diameter ratio so as to provide reasonably sized grafts for the desired application (e.g. rat sciatic gap injury) following rehydration at the time of surgery.

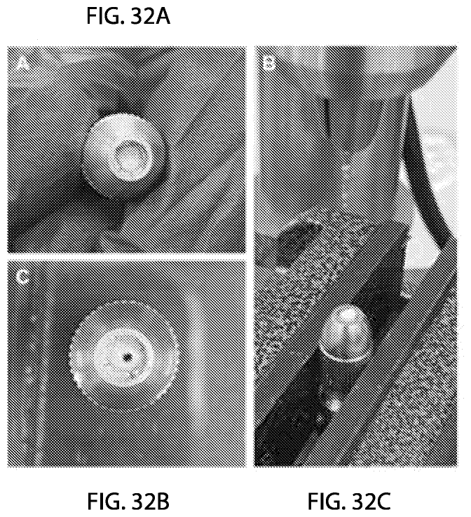

Referring now to FIGS. 32A-C, photographs of a nerve graft-conduit lumen formation are illustrated, consistent with the present inventive concepts. Following the jacketed lyophilization described hereinabove, applicant has attempted multiple lumen creation strategies. As shown in FIG. 32A, one method entailed holding a decellularized, jacketed, and lyophilized nerve segment within a dedicated support system, and creating a lumen using a needle inserted longitudinally throughout the full length of the graft. Following another in vivo study using grafts formed in this manner, applicant found the lumen created by simply displacing tissue with a needle fully collapsed following tissue rehydration; defeating the purpose of a central lumen which is needed to enable the nerve growth cone to extend from the proximal stump along the cylindrical surface of the lumen to the distal stump, in an unobstructed manner in order to facilitate the creation of an initial nerve bridge between the nerve ends. Applicant then pursued additional methods of lumen creation. As shown in FIGS. 32B and C, another method utilizes a small conical carbide burr coupled with a drill press and another assembly to hold the graft. This method produces stable lumens within the PNM-G 2100 by rotationally removing tissue from the lumen via friction. PNM-G 2100 produced using this method were assessed for surgical handling and suture retention strength, demonstrating that the grafts have handling characteristics and mechanical properties (e.g. 1.75 N average suture retention strength) suitable for use in nerve repair applications.

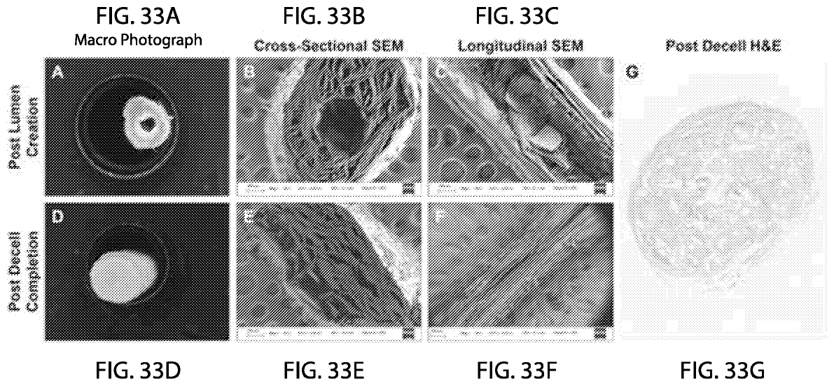

Referring now to FIGS. 33A-G, in-vitro and histological images of a nerve graft-conduit are illustrated, consistent with the present inventive concepts. Applicant has conducted an 8 week animal study that included a 1 cm sciatic nerve gap injury model in rats using PNM-G 2100 that were produced using the optimized decellularization method, the correct source tissue diameter, small pore ePTFE jacketing, and the lumen creation method described hereinabove. The study consisted of four experimental groups: I) PNM-G 2100 without the lumen; II) PNM-G 2100 with the lumen; III) PNM-G 2100 with lumen filled with ECM 120; and IV) inverted autograft as a positive control and standard of care. The inverted autograft control was used as it is known to support nerve regeneration across nerve gap injuries. At study termination, assessment of nerve regeneration motor electrophysiology demonstrated an absence of any appreciable recovery in nerve function in any of the PNM-G 2100 groups, while the nerve autograft was found to recover approximately 20% of the native nerve motor electrophysiologic function. This indicated to applicant that PNM-G 2100 did not support nerve regeneration as initially expected, but rather inhibited it.

Follow-up in vitro and histologic assessments of the PNM-G 2100, as shown in FIGS. 33A-G, demonstrate that despite the use of a dental burr to remove a central core of a lyophilized nerve tissue, the lumen again collapsed following rehydration and implantation due to swelling of the device. Furthermore, an overly compressed tissue structure with longitudinally aligned, but insufficient pore size, was observed throughout the matrix of the graft. It is believed the particular structural features, in combination with the absence of a stable lumen, are responsible for the observed inhibition of nerve recovery.

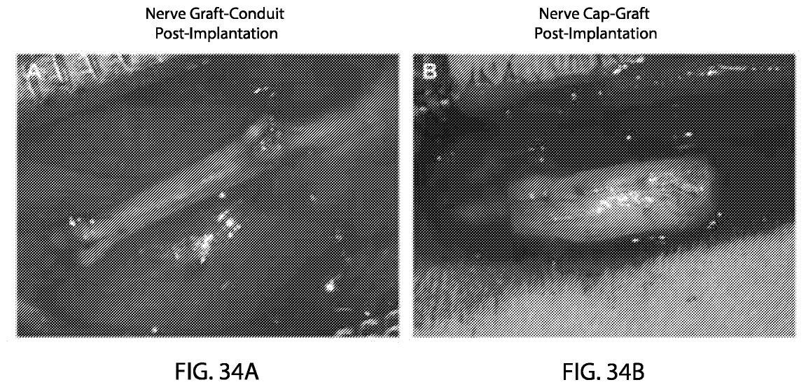

Referring now to FIGS. 34A and B, photographs of a nerve graft-conduit and a nerve cap-graft following implantation at a sciatic nerve injury are illustrated, respectively, consistent with the present inventive concepts. Applicant has performed a study to assess the use of PNM-CAP 2200. PNM-CAP 2200 were implanted into a model of neuroma formation consisting of the removal of a 1 cm portion of the sciatic nerve, and either treatment of its free proximal end with PNM-CAP 2200 or leaving it untreated, which is known to lead to neuroma (e.g. neuroma control). In PNM-CAP 2200 groups, the distal nerve stump is left disconnected as shown in FIG. 34B. Treatment utilizing PNM-G 2100 is shown in FIG. 34A.

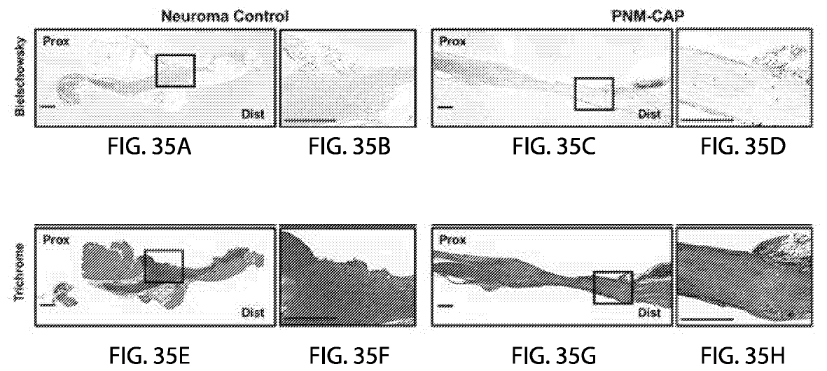

Referring now to FIGS. 35A-H, histological images of a neuroma control, a nerve graft-conduit, and a nerve cap-graft at 8 weeks post-injury in a rodent model are illustrated, consistent with the present inventive concepts. Applicant has conducted studies that demonstrate a progressive loss of myelin starting at the proximal end of PNM-G 2100, PNM-CAP 2200 with near total loss at the distal end. This loss of myelination was accompanied by a progressive decrease in axon number from the proximal to distal end of PNM-G 2100, PNM-CAP 2200. Applicant observed the progressive dissipation of axonal growth was accompanied by increasing replacement of the test article by dense connective tissue, with few, if any, axons observable within these areas. Despite the observed progressive loss of myelin, decrease in axonal number, and replacement of PNM-G 2100, PNM-CAP 2200 with connective tissue, applicant observed the linear orientation and organization of the remaining axons in the native nerve and proximal portion of PNM-G 2100, PNM-CAP 2200 was largely maintained, while lacking the presence of aberrant axonal growth producing neuroma-like lesions. Applicant maintains these results are in direct contrast to the histopathologic findings for the transections without repair (i.e. neuroma positive control) group, which was characterized by a well-organized axonal structure at the proximal aspect of the nerve, transitioning into a tangled and randomly-oriented array of axons distally from the level of the transection. This irregular, tangled mass of axons is consistent with neuroma formation.

As shown in FIGS. 35A-D, Bielschowsky's silver stain, thereby staining axons brown, was performed on the neuroma control, PNM-G 2100, and PNM-CAP 2200. As shown in FIGS. 35E-H, hematoxylin and eosin with Luxol fast blue, thereby staining myelin blue and connective tissue pink, was performed on the neuroma control, PNM-G 2100, and PNM-CAP 2200. Applicant observed the neuroma control is characterized by significant axonal outgrowth from the proximal to distal end as shown in FIGS. 35A and E, with a tangled mass of growing axons and myelin at the distal end as shown in FIGS. 35B and F. Applicant observed the PNM-G 2100, PNM-CAP 2200 groups were characterized by dissipating axonal staining from the proximal to distal end as shown in FIGS. 35D and H, with progressive loss of myelination and replacement of the graft-conduit by connective tissue as shown in FIGS. 35C and G. Applicant further observed the remaining axons have a well aligned organization, and no adverse reactions to the PNM-G 2100, PNM-CAP 2200.

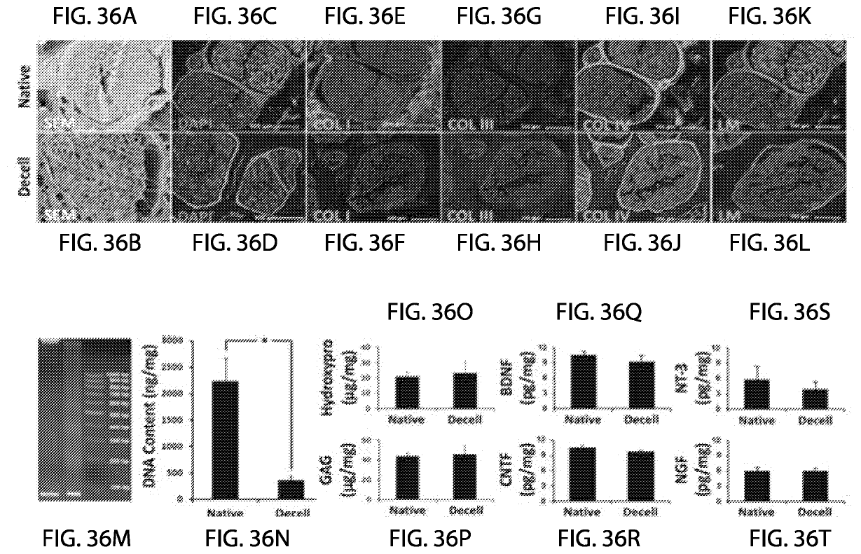

Referring now to FIGS. 36A-T, results demonstrating nerve tissue maintaining multiple structure and functional components following decellularization are illustrated, consistent with the present inventive concepts. Applicant has developed a technology platform based on decellularized porcine nerve tissue to address peripheral nerve injuries and other conditions. While each of the products derived from this platform have different features, intended use, and stage of development, these products share two common characteristics: the same source of nerve material (i.e. young and healthy porcine sciatic nerve); and a decellularization process, which consists of a series of chemical and enzymatic treatments. Applicant has demonstrated the resulting decellularized tissue to be devoid of cellular debris and residual DNA, while retaining the general macroscopic shape and size of the source tissue, as well as maintaining its multi-level microstructure and composition. The decellularized tissue includes a multi-fascicular internal nerve structure, structural and functional proteins like collagen I (as shown in FIGS. 36E and F), collagen III (as shown in FIGS. 36G and H), collagen IV (as shown in FIGS. 361 and J), and laminin (as shown in FIGS. 36K and L), as well as neurotrophic factors including BDNF (as shown in FIG. 36Q), CNTF (as shown in FIG. 36R), NT-3 (as shown in FIG. 36S), and NGF (as shown in FIG. 36T), which are normally harbored in the young healthy tissue and have a critical role in the natural response and recovery from nerve injury. As such, applicant has demonstrated their technology platform comprises a neuro-inductive, biocompatible, and degradable reservoir of neurotrophic factors and nerve structural proteins that can support healthy nerve healing for a variety of applications.

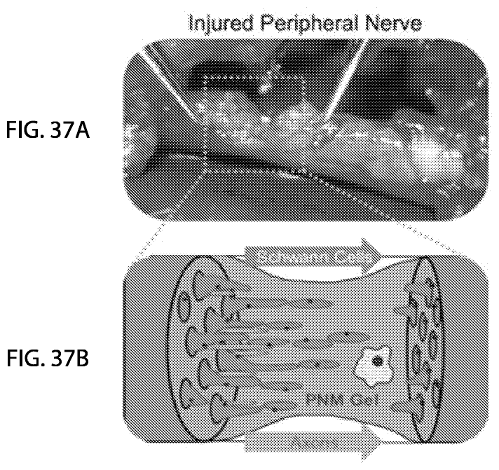

Referring now to FIGS. 37A and B, a photograph and graphical representation of a gel derived from porcine sciatic nerve promoting Schwann cell (SC) proliferation and axon regrowth at the site of a nerve injury are illustrated, respectively, consistent with the present inventive concepts.

Applicant's ECM 120 is derived from a decellularized matrix via solubilization and other processes as described hereinabove. ECM 120 can be delivered at the site of nerve injury and is intended to act both as a scaffold and modulator for native nerve regeneration processes. ECM 120 can have a critical impact on the nerve repair and functional recovery following multiple types of acute nerve injuries. ECM 120 has been found to support Schwann cell (SC) migration, axon elongation, and a transition from pro- to anti-inflammatory macrophage phenotype in a manner which has been shown to promote effective nerve healing. As shown in FIGS. 37A and B, delivery of ECM 120 to the site of an injured peripheral nerve can promote SC proliferation and axon regrowth.

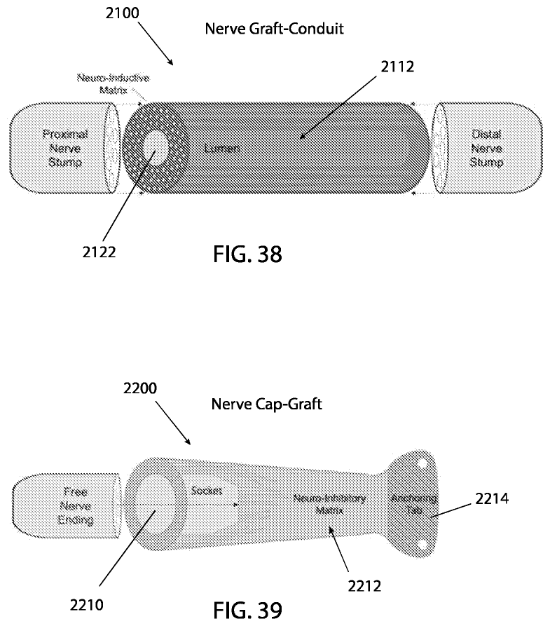

Referring now to FIG. 38, a graphical representation of a nerve graft-conduit comprising a peripheral nerve matrix is illustrated, consistent with the present inventive concepts. Applicant's ECM 120 can be formed into a nerve graft-conduit, PNM-G 2100, configured to support nerve regrowth at the site of a nerve injury. In some embodiments, PNM-G 2100 is configured to serve as a physical bridge for nerve gap injuries. PNM-G 2100 can comprise an internal central lumen running along its full length, and the lumen can be surrounded by a neuro-inductive matrix with longitudinally aligned pores; these features are critically important in the initial phases of nerve regeneration as they allow a low resistance path for budding nerve growth and directional cues (i.e. neuro-conductive), which combined with neurotrophic factors, can lead to faster and enhanced regeneration of the tissue (i.e. neuro-inductive). Rather than solubilization, PNM-G 2100 utilizes a series of mechanical and physical treatments resulting in a regular tubular geometry with an open lumen spanning the full length of the graft, with a neuro-inductive matrix surrounding the lumen. Therefore, PNM-G 2100 can foster nerve regeneration between two nerve endings by providing a template for axonal regeneration via the internal lumen and neuro-inductive matrix.

Referring now to FIG. 39, a graphical representation of a nerve cap-graft comprising a peripheral nerve matrix is illustrated, consistent with the present inventive concepts. Applicant's ECM 120 can be formed into a cap with an anchoring tab, such as PNM-CAP 2200 including anchoring tab 2214, configured to inhibit neuroma formation. In some embodiments, PNM-CAP 2200 is configured to prevent the development of painful neuroma, a common condition following limb amputation caused by an aberrant nerve tissue growth at the severed free nerve ending. Inhibition of neuroma formation can be obtained by creating a socket 2210 within PNM-CAP 2200 to receive a free nerve ending and a surrounding the nerve ending with a neuro-inhibitory matrix 2212, permitting the longitudinally aligned growth of the nerve, but progressively slowing the axon growth to a stop without formation of neuroma. The inhibition is achieved by exhausting the regenerative potential of the sprouting axons while extending their growth into progressively more restrictive, longitudinally-oriented pores.

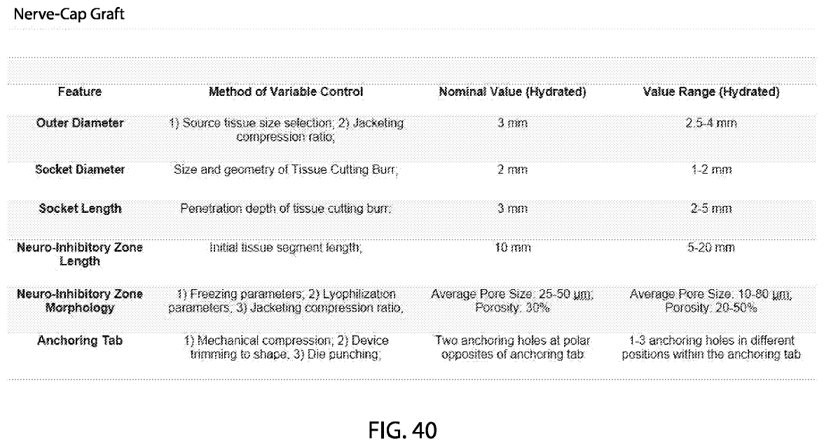

Referring now to FIG. 40, a table containing feature development parameters of a nerve cap-graft is illustrated, consistent with the present inventive concepts. Applicant has identified six PNM-CAP 2200 design features: outer diameter; socket 2210 diameter; socket 2210 length; neuro-inhibitory matrix 2212 length; neuro-inhibitory matrix 2212 morphology; and anchoring tab 2214.

The outer diameter of PNM-CAP 2200 can be configured to provide sufficient suture retention strength and separation from surrounding tissues without compromising permeability to nutrients or creating excessive bulk. In some embodiments, PNM-CAP 2200 comprises an outer diameter of between 2.5 mm and 4 mm, such as approximately 3 mm, when hydrated. Applicant can control the nerve outer diameter by modifying the nerve tissue harvest process, such as by stratifying and selecting segments from well-defined anatomical locations to provide several predictable sizes. Nerve tissue can be harvested at the main trunk of the porcine sciatic nerve, representing one of the largest diameter nerves available. The sciatic nerve branches into the tibial, common peroneal, and sural nerves. Each nerve branch can be harvested at a specified length from the branching point, or at secondary branching points, to achieve control in nerve diameter and fascicular complexity. Additionally, different size jacketing of the nerve can be utilized to provide a consistent outer diameter to the end product. The outer diameter of PNM-CAP 2200 can be measured via a vernier caliper and image-based technique for the dry and rehydrated states, respectively.

The socket 2210 diameter of PNM-CAP 2200 can be configured to allow a clinician (e.g. surgeon) to insert the severed proximal free nerve ending into the product and secure it via standard sutures. In some embodiments, PNM-CAP 2200 comprises a socket 2210 diameter of between 1 mm and 2 mm when hydrated. The diameter of socket 2210 can be controlled by removing tissue from the nerve in a frozen or lyophilized state to form a central bore via the use of carbide burrs or drill bits mounted in the drill press setup as described hereinabove, and whereby different burr shapes and sizes can be employed. In some embodiments, the diameter of socket 2210 can be controlled by removing tissue from the nerve in a frozen or lyophilized state to form a central bore via the use of laser ablation, in which the specific size and shape of socket 2210 created is controlled by a user-defined dimension vector graphic. Alternatively, the diameter of socket 2210 can be controlled by removing tissue from the nerve in a frozen or lyophilized state to form a central bore via the use of electrosurgery or ultrasound cutting, whereby a different tip size can be employed. The diameter of socket 2210 can be measured using a set of pin gauges when PNM-CAP 2200 is in a dry state. Additional visualization of the diameter of socket 2210 can be obtained by tissue surface marking dye and resin casting of the socket. Additional examination under SEM and Micro Computed Tomography (Micro CT) can be performed to further characterize the final geometry of socket 2210 and the morphology of the surface of neuro-inhibitory matrix 2212.

The length of socket 2210 of PNM-CAP 2200 can be configured to allow sufficient overlap with the proximal free nerve ending to secure the nerve in position, as well as protect it from surrounding tissue, while leaving sufficient space for sutures. In some embodiments, socket 2210 comprises a length of between 2 mm and 5 mm, such as approximately 3 mm, when hydrated. The length of socket 2210 can be controlled by the depth of burr penetration into PNM-CAP 2200 using the same drill press setup as described hereinabove. The length of socket 2210 can be controlled using laser ablation by altering settings of: wavelength; power; pulse width; speed; number of passes; vertical distance between tissue and head of laser beam; and combinations of these. The length of socket 2210 can be controlled using electrosurgery or ultrasound cutting by altering: power; frequency/waveform, current; rate and regimen of tip longitudinal travel; and combinations of these. The length of socket 2210 can be measured using a depth micrometer while the PNM-CAP 2200 is in a dry state. Additional visualization of the diameter of socket 2210 can be obtained by tissue surface marking dye and resin casting of socket 2210.

The length of neuro-inhibitory matrix 2212 of PNM-CAP 2200 can be configured to act as a substrate for progressive inhibition of nerve outgrowth in a well-organized tissue architecture. In some embodiments, PNM-CAP 2200 comprises a neuro-inhibitory matrix 2212 length of between 5 mm and 20 mm, such as approximately 10 mm, when hydrated. The length of neuro-inhibitory matrix 2212 can be controlled by providing a decellularized and lyophilized tissue of known length, with standard socket 2210 depth, with the remainder of the length of the product representing neuro-inhibitory matrix 2212 and anchoring tab 2214. Previous studies utilizing acellular autografts have shown that, when axon-permeable materials are used, a significant graft-conduit length (e.g. 5 cm) is needed to terminate axonal growth. However, applicant has demonstrated axonal exhaustion begins at the proximal end of the graft-conduit with axonal growth termination in <10 mm length.

The porosity of neuro-inhibitory matrix 2212 and pore size of PNM-CAP 2200 can be configured to allow a limited number of axons to grow within each pore and travel distally through the inhibitory matrix. In some embodiments, PNM-CAP 2200 comprises an average pore size of between 10 μm and 80 μm, such as between 25 μm and 50 μm, when hydrated. In some embodiments, PNM-CAP 2200 comprises a porosity of between 20% and 50%, such as approximately 30%, when hydrated. The porosity and pore size of neuro-inhibitory matrix 2212 can be controlled by controlling ECM 120 freezing conditions (e.g. freezing front direction, rate, etc.) to create water crystals that result in pores with distinct sizes and alignment prior to lyophilization, as previously described. Lyophilization parameters (e.g. vacuum level, primary and secondary drying temperature rates, etc.) also have distinct effects upon porosity and pore size. The porosity of neuro-inhibitory matrix 2212 can be modified along the length of the PNM-CAP 2200 by providing varying levels of mechanical compression to the inhibitory matrix throughout the manufacturing process (e.g. tapered jacketing, conical or cylindrical compression mold), thereby creating an increasingly dense structure from the proximal to distal end of the PNM-CAP 2200. By modifying the porosity along the length of neuro-inhibitory matrix 2212, the regenerative potential of the sprouting axons moving toward the distal portion of the device by confining and compartmentalizing them into tapering, longitudinally-oriented pores can be impacted. Additionally, pore size, geometry, alignment, and interconnectivity can be investigated using a modified vascular corrosion casting process. The casting process can include perfusing the tissue using a commercially available radiopaque methylmethacrylate-based casting material kit. Following polymerization, the tissue-polymer composites are treated with a caustic solution to remove all tissue prior to imaging. Imaging can be performed under SEM, or using Micro CT with 3D reconstruction as necessary to fully visualize and quantify the resultant structures.

Anchoring tab 2214 of PNM-CAP 2200 can be configured to allow firm surgical fixation to surrounding tissue to maintain desired alignment. The anchoring tab 2214 can be produced by applying mechanical compression with a bench press to one end of the PNM-CAP 2200, trimming anchoring tab 2214 to the desired shape and size via a punch and die, and punching two holes on either end of the tab to allow anchoring. Anchoring tab 2214 is configured to remain intact (i.e. compressed) during hydration to allow it to serve its function at the time of implantation. Anchoring tab 2214 is further configured to possess sufficient suture retention strength for its intended application. In some embodiments, anchoring tab 2214 is crosslinked using physical (e.g. heat) or chemical (e.g. carbodiimide, glutaraldehyde) processes to prevent swelling during hydration, or to increase suture retention strength.

Referring now to FIG. 41, a table containing feature development parameters of a nerve graft-conduit is illustrated, consistent with the present inventive concepts. Applicant has identified five PNM-G 2100 design features: outer diameter; lumen diameter; overall length; neuro-inductive matrix morphology; and device degradation rate.

In some embodiments, PNM-G 2100 comprises an outer diameter of between 2 mm and 3 mm when hydrated. The nerve outer diameter can be controlled by modifying the nerve tissue harvest process, such as by stratifying and selecting segments from well-defined anatomical locations to provide several predictable sizes. Nerve tissue can be harvested at the main trunk of the porcine sciatic nerve, representing one of the largest diameter nerves available. The sciatic nerve branches into the tibial, common peroneal, and sural nerves. Nerve tissue can be harvested from a specific one of these branches at a specified length from the branching point, or at secondary branching points, to achieve control in features such as nerve diameter and fascicular complexity. Additionally, different size jacketing of the nerve can be utilized to provide a consistent outer diameter to the end product. The outer diameter of PNM-G 2100 can be measured via a vernier caliper and image-based technique for the dry and rehydrated states, respectively.

In some embodiments, PNM-G 2100 comprises a lumen diameter of between 1 mm and 2 mm when hydrated. The diameter of lumen 2122 can be controlled by removing tissue from the nerve in a frozen or lyophilized state to form a central bore via the use of carbide burrs or drill bits mounted in the drill press setup as described hereinabove, and whereby different burr shapes and sizes can be employed. Alternatively, the diameter of lumen 2122 can be controlled by removing tissue from the nerve in a frozen or lyophilized state to form a central bore via the use of laser ablation, in which the specific size and shape of the lumen created is controlled by a user-defined dimension vector graphic. Alternatively, the diameter of lumen 2122 diameter can be controlled by removing tissue from the nerve in a frozen or lyophilized state to form a central bore via the use of electrosurgery or ultrasound cutting, whereby different tip size can be employed. The diameter of lumen 2122 can be measured using a set of pin gauges when PNM-G 2100 is in a dry state. Additional visualization of the diameter of lumen 2122 can be obtained by tissue surface marking dye. Additional examination under SEM and Micro CT can be performed to further characterize the lumen final geometry and the morphology of the lumen-matrix surface.

In some embodiments, PNM-G 2100 comprises an overall length of between 15 mm and 30 mm when hydrated. The overall length can be controlled by modifying the initial tissue segment length. The overall length can be controlled by trimming PNM-G 2100 prior to implantation.

In some embodiments, PNM-G 2100 comprises a neuro-inductive matrix 2112 comprising an average pore size of between 80 μm and 250 μm, such as between 100 μm and 200 μm, when hydrated. In some embodiments, PNM-G 2100 comprises a neuro-inductive matrix 2112 comprising a porosity of between 50% and 80%, such as approximately 60%, when hydrated. The porosity and pore size of the neuro-inductive matrix 2112 can be controlled by modifying: one, two, or more freeze casting parameters; one, two, or more laser ablation parameters; and/or one, two, or more porogen leaching parameters.

In some embodiments, PNM-G 2100 comprises a degradation rate of between 30 days and 90 days when hydrated. Histological images (not shown) indicate PNM-G 2100 maintains a continuous structure 30 days post-implantation. The degradation rate can be controlled by modifying PNM-G 2100 to comprise a higher density morphology, chemical cross-linking, and/or physical cross-linking.

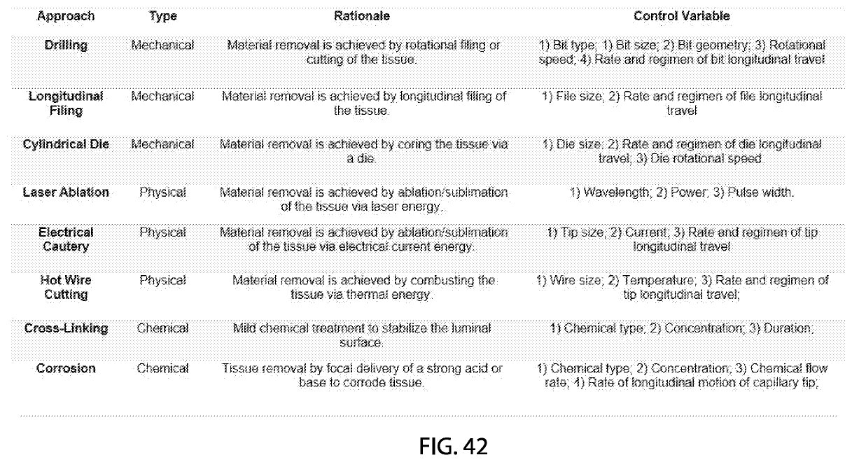

Referring now to FIG. 42, a table containing nerve graft-conduit lumen formation strategies is illustrated, consistent with the present inventive concepts. As described hereinabove, application initially observed unsuccessful lumen stability upon PNM-G 2100 rehydration and implantation, leading to neuro-inhibitory rather than neuro-inductive performance. The collapse of the lumen resulted from the use of methods which primarily displaced, rather than removed, the matrix. Following rehydration, the radially-displaced matrix expanded inward toward the center of the lumen causing closure. As such, applicant intends to evaluate various lumen formation strategies to ensure PNM-G 2100, upon rehydration and subsequent implantation, maintains its intended diameter along the full length of the device. The lumen should be sized to allow rapid creation and propagation of the nerve growth cone from the proximal stump and support the formation of a tissue bridge across the device in vivo. Also, the lumen should be designed such that the wall of the PNM-G 2100 retains sufficient suture strength for use in nerve gap repair applications, but also retains sufficient kink resistance to prevent the blockage of nerve growth through the lumen.

As shown in FIG. 42, applicant intends to evaluate a diverse set of material removal strategies including mechanical (e.g. burr drilling, etc.), physical (e.g. laser ablation, electrosurgery, ultrasound cutting, etc.), and chemical (e.g. focal corrosion, etc.). Applicant will pair these removal strategies with tools to hold the PNM-G 2100 in place during the lumen formation process, and to center, size, and orient the lumen's longitudinal axis within the volume of the PNM-G.

In some embodiments, lumen 2122 is formed within PNM-G 2100 via drilling, such as burr drilling. Material can be removed by rotational filing or cutting of the tissue therein. Control variables can comprise one or more of the following: bit type; bit size; bit geometry; rotational speed; rate and regiment of bit longitudinal travel.

In some embodiments, lumen 2122 is formed within PNM-G 2100 via longitudinal filing. Material can be removed by longitudinal filing of the tissue therein. Control variables can comprise one or more of the following: file size; rate and regimen of file longitudinal travel.

In some embodiments, lumen 2122 is formed within PNM-G 2100 via a die, such as cylindrical die. Material can be removed via coring the tissue therein via a die. Control variables can comprise one or more of the following: die size; rate and regimen of die longitudinal travel; die rotational speed.

In some embodiments, lumen 2122 is formed within PNM-G 2100 via ablation, such as laser ablation. Material can be removed via ablation/sublimation of the tissue therein using laser energy. Control variables can comprise one or more of the following: laser type (e.g. carbon dioxide, argon ion, Er—YAG (erbium-doped yttrium aluminum garnet), Nd—YAG (neodymium-doped yttrium aluminum garnet); wavelength; power; pulse width; speed; number of passes; and/or vertical distance between tissue and head of laser beam.

In some embodiments, lumen 2122 is formed within PNM-G 2100 via electrical surgery cutting technique (e.g. cautery). Material can be removed via ablation/sublimation of the tissue therein using electrical current energy. Control variables can comprise one or more of the following: tip size; current; rate and regimen of tip longitudinal travel.

In some embodiments, lumen 2122 is formed within PNM-G 2100 via hot wire cutting. Material can be removed via combusting the tissue therein using thermal energy. Control variables can comprise one or more of the following: wire size; temperature; rate and regimen of tip longitudinal travel.

In some embodiments, lumen 2122 is formed within PNM-G 2100 via cross-linking. Material can be removed via mild chemical treatment to stabilize the luminal surface. Control variables can comprise one or more of the following: chemical type; concentration; duration.

In some embodiments, lumen 2122 is formed within PNM-G 2100 via corrosion. Material can be removed via focal delivery of a strong acid or base to corrode the tissue therein. Control variables can comprise one or more of the following: chemical type; concentration; chemical flow rate; rate of longitudinal motion of capillary tip.

In some embodiments, two or more of the above methods are used in succession to form the lumen 2122 to the desired specifications with minimal damage to surrounding tissue. One method may be repeated two or more times with same or different control variables to form the lumen 2122 to the desired specifications.

In some embodiments, support assembly 300 includes a protrusion to be inserted into a newly formed lumen 2122 before rehydration, which can increase the stability of lumen 2122. In some embodiments, lumen 2122 is filled with a material (e.g. PNM) to enhance stability of lumen 2122, mechanical properties of PNM-G 2100, and neuroinductive properties of PNM-G 2100.

Following lumen creation, the devices will undergo hydration protocols that simulate use in the operating theater pre-implantation (e.g. 10 minutes in cold sterile saline), and extended in vivo hydration (e.g. 24 hours in sterile saline at 37° C.) to assess the stability of the lumen. Pre- and post-hydration lumen diameters at different locations along the length of the PNM-G 2100 will be assessed by fixing the rehydrated PNM-G 2100 in 10% formalin and embedding in paraffin, and serial 20 μm sections will be cut via a microtome and stained with hematoxylin and eosin. Image analysis applied to the histologic images will be used to measure geometrical features of the lumen. SEM and Micro CT may be used to assess lumen geometry at multiple levels of the graft-conduit as needed, and as described above.

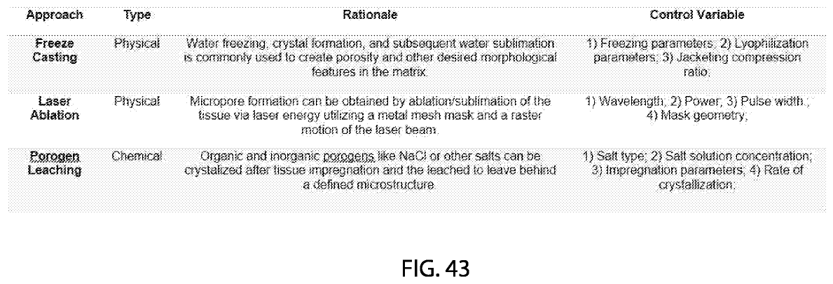

Referring now to FIG. 43, a table containing nerve graft-conduit matrix morphology control strategies is illustrated, consistent with the present inventive concepts. As described hereinabove, applicant initially observed that the matrix of the PNM-G 2100 did support and induce axonal growth (i.e. neuro-inductive). However, the matrix was found to be neuro-inhibitory. Therefore, in addition to lumen 2122 as described herein, the annular matrix surrounding it represents a second functional element critical to the success of PNM-G 2100. In order to convert the existing matrix from neuro-inhibitory to neuro-inductive, it is believed the matrix must have suitable porosity, pore size distribution and interconnectedness, longitudinal pore alignment, and cell adhesion properties to freely allow and support the infiltration of SC and neurites during the nerve regeneration process from the proximal toward the distal portion of the device.

As shown in FIG. 43, in order to create such a neuro-inductive annular structure, applicant intends to evaluate a diverse set of morphology control strategies including physical (e.g. freeze casting, laser ablation, etc.), and chemical (e.g. porogen leaching, etc.) to affect both the size, density, and alignment of the pores within PNM-G 2100 matrix.

In some embodiments, PNM-G 2100 matrix morphology is controlled via freeze casting. Water freezing, crystal formation, and subsequent water formation can be used to create porosity and other desired morphological features within the matrix. Control variables can comprise one or more of the following: freezing parameters; lyophilization parameters; and jacketing compression ratio.

In some embodiments, PNM-G 2100 matrix morphology is controlled via ablation, such as laser ablation. Micropore formation can be obtained via ablation/sublimation of the tissue therein using laser energy in conjunction with a metal mesh mask and a raster motion of the laser beam. Control variables can comprise one or more of the following: laser type; wavelength; power; pulse width; speed; number of passes; vertical distance between tissue and head of laser beam; and mesh mask geometry.

In some embodiments, PNM-G 2100 matrix morphology is controlled via porogen leaching. Organic and/or inorganic porogens (e.g. NaCl, other salts) can be crystallized after impregnation within the tissue and then leached to leave behind a defined microstructure. Control variables can comprise one or more of the following: salt type; salt solution concentration; impregnation parameters; rate of crystallization.

Based on the results obtained with the individual strategies described hereinabove, applicant may further evaluate using combinations of these strategies. The desired outcome includes a larger pore size and porosity than those of PNM-CAP 2200. In some embodiments, PNM-G 2100 comprises an initial nominal average pore size of between 100 μm and 200 μm, and a porosity of approximately 60%.

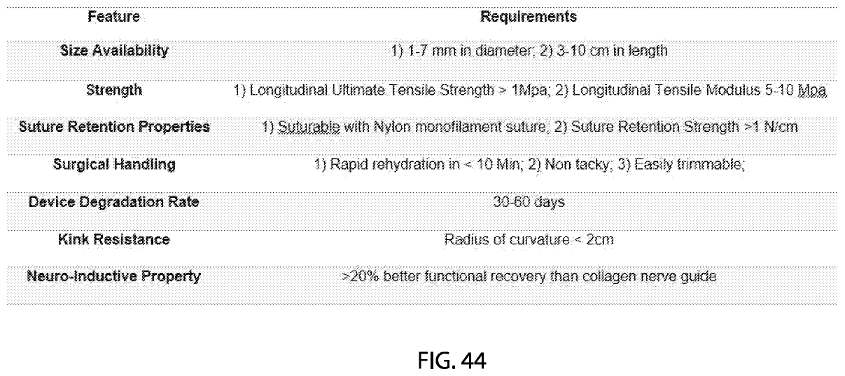

Referring now to FIG. 44, a table containing nerve graft-conduit clinical requirements is illustrated, consistent with the present inventive concepts. As described hereinabove, applicant has identified features of PNM-G 2100 internal lumen (e.g. lumen 2122) and matrix (e.g. matrix 2112) morphology to obtain desired neuro-inductive properties for use in a rodent model. However, applicant recognizes the clinical PNM-G 2100 requires additional features to meet clinical requirements.

For clinical applications, the PNM-G 2100 is available in various diameters and lengths. PNM-G 2100 can comprise an outer diameter of between 1 mm and 7 mm. In some embodiments, the PNM-G 2100 comprises an outer diameter configured to accommodate a 2 mm nerve (e.g. digital nerves). In some embodiments, PNM-G 2100 comprises an outer diameter configured to accommodate a 4 mm nerve (e.g. distal ulnar or radial nerve). PNM-G 2100 can comprise an overall length of between 3 mm and 10 mm. In some embodiments, PNM-G 2100 comprises an overall device length of at least 3 cm to allow trimming and application across a reasonable range of clinically relevant nerve injuries.

For clinical applications, PNM-G 2100 is configured to allow for growth of nerve tissue within PNM-G 2100. As such, PNM-G 2100 can be permeable and supportive to the nerve growth cone, thereby allowing cells to move longitudinally across the whole length of the device to ultimately bridge the two nerve stumps. This can be achieved with a hollow lumen 2122 or by filling such lumen with ECM 120.

For clinical applications, PNM-G 2100 is configured with sufficient strength to sustain the tensions, flexions, and torsions between two nerve stumps as a result of physiological motion.

For clinical applications, PNM-G 2100 comprises sufficient suture retention strength. In some embodiments, PNM-G 2100 comprises a suture retention strength >1N/cm. In some embodiments, PNM-G 2100 is compatible for use with nylon monofilament sutures.

For clinical applications, PNM-G 2100 can comprise one, two, or more features to support surgical handling. In some embodiments, PNM-G 2100 is configured for rapid rehydration, such as rehydration in less than 10 mins. In some embodiments, PNM-G 2100 comprises a non-tacky surface. In some embodiments, PNM-G 2100 is easily trimmable by a clinician (e.g. surgeon).

For clinical applications, PNM-G 2100 comprises degradation rates compatible with the speed of nerve growth, such as 1 mm/day. For example, for a 2 cm device, the material will need to remain in place for a minimum of 20 days. In some embodiments, PNM-G 2100 is configured to degrade between 30 days and 60 days. The degradation rate is slower than the speed of nerve regeneration in order to maintain physical guidance between the proximal and distal nerve stumps.

For clinical applications, PNM-G 2100 comprises a kink resistance to prevent closure of lumen 2122 during flexion, thereby inhibiting and/or disrupting nerve regeneration and growth. In some embodiments, PNM-G 2100 comprises a radius of curvature <2 cm.

For clinical applications, PNM-G 2100 comprises neuro-inductive properties to enhance its ability to support nerve remodeling and regeneration (as compared with collagen and/or synthetic nerve conduits). In some embodiments, PNM-G 2100 comprises >20% greater functional recovery than collagen nerve conduits.

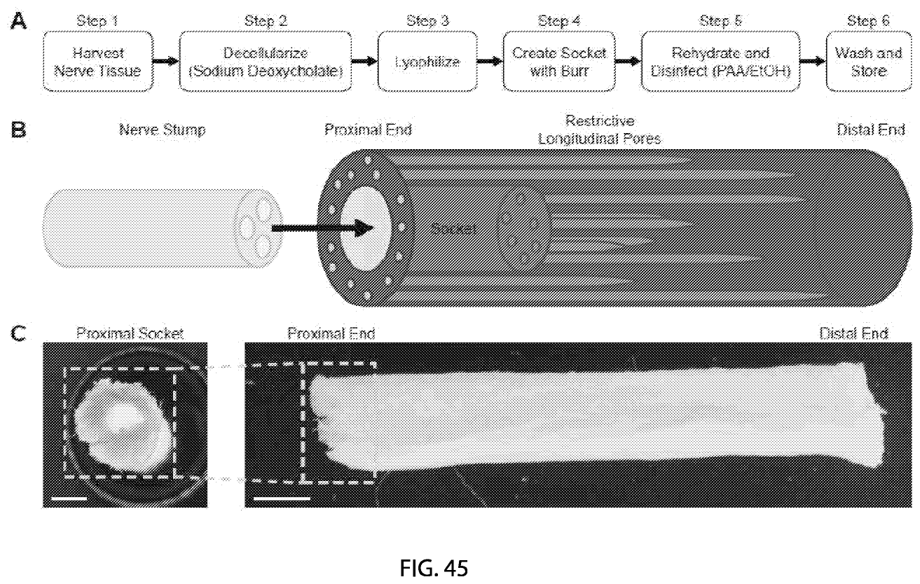

Referring now to FIG. 45 through FIG. 53, various graphical representations, photographs, histological images, and graphs regarding the development of an acellular nerve cap-graft xenograft for neuroma prevention are illustrated, consistent with the present inventive concepts. Neuroma formation following limb amputation is a prevalent and debilitating condition that can deeply affect quality of life and productivity. Several approaches exist to prevent or treat neuromas; however, no approach is either consistently reliable or surgically facile, with high rates of neuroma occurrence and/or recurrence. The present study describes the development and testing of a xenogeneic nerve cap-graft graft made from decellularized porcine nerve. The grafts were tested in vitro for cellular removal, cytotoxicity, mechanical properties, and morphological characteristics. The grafts were then tested in rat sciatic nerve gap reconstruction and nerve amputation models for 8 weeks. Gross morphology, electrophysiology, and histopathology assessments were performed to determine the ability of the grafts to limit pathologic nerve regrowth. In vitro testing showed well decellularized and demyelinated nerve cap-graft graft structures without any cytotoxicity from residual reagents. The grafts had a proximal socket for the proximal nerve stump and longitudinally oriented internal pores. Mechanical and surgical handling properties suggested suitability for implantation as a nerve graft. Following 8 weeks in vivo, the grafts were well integrated with the proximal and distal nerve segments without evidence of fibrotic adhesions to the surrounding tissues or bulbous outgrowth of the nerve. Electrophysiology revealed absence of nerve conduction within the remodeled nerve cap-graft grafts and significant downstream muscle atrophy. Histologic evaluation showed well organized, but limited axonal regrowth within the grafts without fibrous overgrowth and neuromatous hypercellularity. These results provide proof of concept for a novel xenograft-based approach to neuroma prevention.

Neuromas are pathologic, non-neoplastic, nerve lesions resulting from aberrant and disorganized axonal outgrowth and cellular proliferation caused by nerve injury. These lesions originate from a physiologic outgrowth to restore continuity of the affected nerve; however, when the regenerating bridge fails to reach the distal target, a tangled bulbous mass forms, with axonal trapping and leading to neuropathic pain. This condition is frequently chronic, physically debilitating, and results in reduction of quality of life. Painful neuromas reduce the mobility of those affected, and may lead to psycho-social conditions such as depression and withdrawal. While the full spectrum of etiologies and the underlying mechanisms of neuroma pain remain sub-optimally defined, severe neuropathic pain may occur with acute and chronic nerve injuries, limb amputations, and iatrogenic nerve injuries.

It is estimated that neuromas develop in 3-5% of all peripheral nerve injuries, with rates approaching 85% in lower limb amputees. 185,000 amputation procedures are performed annually in the U.S. In this population, a single limb amputation can lead to multiple neuromas, as multiple nerves are transected in the procedure. This multiplicity of risk leads to the high incidence after amputation as well as the difficulty in anatomical diagnosis and successful treatment.

Surgical interventions to treat or prevent neuroma formation when nerve functional recovery is not attainable, as is the case following amputation, include neuroma excision, traction neurectomy, and various forms of nerve coaptation or nerve burial. Despite the availability of multiple surgical approaches, there is no clinical ‘gold-standard’ for treatment of neuromas, and success depends on both the nature and location as well as careful patient selection. Furthermore, up to 20-30% of neuromas are refractory to these treatments regardless of the type of surgical intervention, and reoperation rates for neuroma are as high as 65%.

Additional approaches to treat and prevent neuromas include the application of a device (cap or allograft) to the end of the transected nerve. These devices can be made of synthetic or biologic materials and appear to attenuate painful neuroma formation, potentially by isolating free sensitized axons. While the cap devices have shown promising results in preclinical studies, they have yet to be widely adopted, as the limited clinical evidence available suggests that nerve cap-grafting remains inferior to existing surgical approaches. Recent studies have demonstrated that application of an acellular allograft provides a template for termination of the axonal growth potential within the length of the device; while this approach has shown promise, the length (i.e. 5 cm) of allograft required to exhaust the regenerative potential of the injured nerve creates the need for a more extensive surgical site.

In this exploratory study, we developed and tested a novel nerve cap-graft graft made of decellularized porcine nerve tissue. The goal was to assess a novel device for possible improvement upon the existing approaches for neuroma prevention using a clinically translatable product. We have previously developed a method for decellularization of porcine nerve tissue involving multiple chemical reagents and washes. Through a similar chemical decellularization process, native porcine nerves were cleared of cells, cellular debris/DNA, and myelin while retaining a native-like structure. The removal of cellular components was assessed through a semiquantitative histology-based analysis. The resulting decellularized nerve matrix was quantitatively assessed for cytotoxicity to demonstrate the removal of potentially cytotoxic reagents used for the decellularization process. Additional processing of the decellularized nerve tissues was performed to create axon growth-restrictive longitudinal porosity within the graft and an interface for connecting the graft to the nerve end. The resulting grafts were then implanted in a 10 mm rat sciatic nerve gap model for 8 weeks to explore whether such a cap graft approach could be used to inhibit nerve growth, which we believe is an important step towards the prevention of neuroma. Additional pilot studies in a model of nerve amputation were then performed, further demonstrating the utility of the grafts for neuroma prevention.

Materials and Methods. Experimental Overview. The nerve cap-graft graft developed in this study is a cylindrical tissue graft derived from porcine sciatic nerve extracellular matrix. The grafts have longitudinal pores with a diameter that is sufficient to allow initial axonal ingrowth, but too restrictive to allow nerve regeneration throughout the graft. The grafts are intended to be secured to the proximal stump of a transected nerve using standard surgical techniques. The nerve cap-grafts were tested both in vitro and in vivo. In vitro testing was used to assess the critical properties required for implantation as a nerve cap-graft graft. In particular, the degree of tissue decellularization, the potential for cytotoxicity from residual reagents used during decellularization, and the structural and mechanical properties of the grafts were assessed. The grafts were then assessed in vivo in a rat model of sciatic nerve transection to explore the ability of the nerve cap-graft graft to limit nerve growth in a controlled fashion without eliciting neuroma.

Preparation of Nerve cap-graft Grafts. The main trunk of the sciatic nerve with its branches was harvested from the upper thigh of market weight male Landrace X Yorkshire pigs in a commercial abattoir. The nerves were frozen in PBS and stored at −80° C. until use. The tissues were then thawed at 4° C. and rinsed prior to mechanical removal of excess connective and adipose tissue. Following cleaning, nerve segments of approximately 2.5 mm in diameter were isolated from the rest of the tissue and sectioned into 15 mm segments. These segments were immersed in a stirred beaker containing a solution of 4% sodium deoxycholate (w/v) for 8 hours at room temperature to remove cellular content. The tissue was then washed repeatedly with water to remove residual reagent.

The decellularized nerve segments were then pulled through the lumen of pre-cut polytetrafluoroethylene (PTFE) tubing with inner diameters smaller than those of the nerve segments (2.1 mm internal diameter, and 1 cm in length) until the length of each nerve segment was centered within the PTFE tube. The excess tissue protruding from each end of the tubing helped securing the nerve tissue to the tubing during the lyophilization process. The PTFE tubing containing segments of decellularized nerve were then frozen at −20° C., and lyophilized in a benchtop lyophilizer (Labconco Freezone 2.5 plus) for 72 hours.

Following lyophilization, the two bulging ends of tissue were trimmed with a scalpel blade and a socket of 1.2 mm internal diameter and 2 mm in length was created at the proximal end with a cylindrical diamond burr. The nerve cap-graft grafts were then removed from their PTFE supports, rehydrated in water, and placed back into a stirred beaker with a disinfecting solution of 0.1% peracetic acid and 4% ethanol at room temperature for 2 hours. The nerve cap-graft grafts were then washed with sterile PBS and water in a stirred beaker at room temperature for 15 minutes per wash. A total of 2 PBS and 2 water washes were performed to remove the disinfecting solution from the tissue and to neutralize the pH of the grafts. The nerve cap-graft grafts were then stored in sterile PBS at 4° C. until use. A schematic outline of the decellularization process, nerve cap-graft graft structure and features, and representative images of the nerve cap-graft graft are shown in FIG. 45.

FIG. 45 (Preparation of Nerve cap-graft Graft: A. Summary of Nerve cap-graft Graft processing steps. B. Representative diagram of Nerve cap-graft Graft features. C. Appearance of Nerve cap-graft Graft after Step 4. Scale=1 mm).

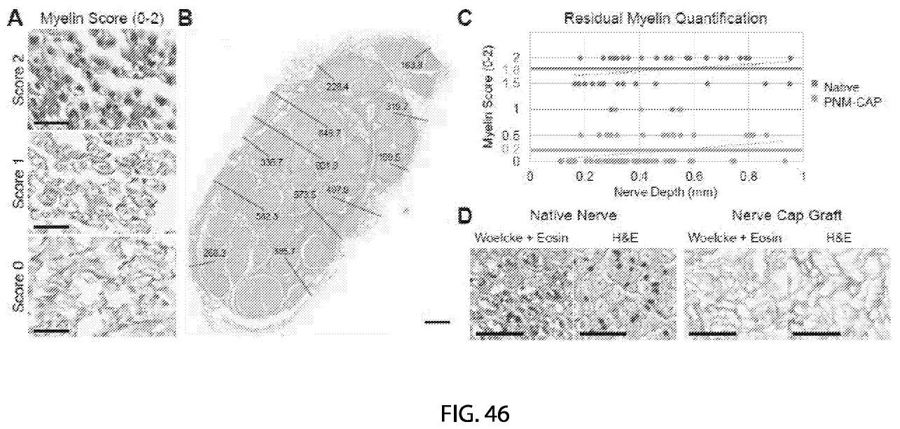

Decellularization Analysis. Fresh nerve tissue segments (1 cm in length; control) and decellularized nerve cap-graft grafts were assessed for myelin and cellular content via histologic analysis as a metric of decellularization. Briefly, tissue samples were fixed in 10% neutral buffered formalin solution, processed, and embedded in paraffin blocks. Cross-sections of 5 μm in thickness were then cut every 2 mm along the longitudinal direction of each sample (5 cross-sections per graft), placed on a single slide, and stained with either Woelcke's method or hematoxylin and eosin (H&E) for the identification of myelin and nuclei, respectively. Samples were analyzed semi-quantitatively using a myelin scale (i.e. 0—minimal to no myelin; 1—reduced and/or fragmented myelin; 2—native-like myelin; FIG. 46A). In each analyzed Woelcke-stained section, 12 separate fascicles were identified and quantified for myelin score along with their radial distance from the outer surface of the nerve. A representative image of the analysis performed on an individual section is shown in FIG. 46B. The distribution of myelin scores as a function of radial distance was plotted for each graft in order to assess the amount of myelin present in the graft as a function of location within the graft. Absence of cellular content was confirmed qualitatively at mid-graft by H&E stain. The mid-graft location was analyzed as it represents the most challenging location for the mass-transport of the reagents during tissue decellularization.

FIG. 46 (Nerve cap-graft Graft decellularization analysis. A. Semi quantitative scale for myelin quantification using Woelcke and Eosin stains at 10× magnification. Black staining indicates myelin. Scale=25 μm. B. Representative quantification of residual myelin in Native Nerve at 2.5× magnification. Scale=250 μm. C. Quantification of residual myelin as a function of nerve depth and group. Dotted line is linear regression, solid line is group average. D. Representative appearance of Woelck+Eosin (left) and H&E (right) for Native Tissue and Nerve cap-graft Graft groups at 40× magnification. Scale=25 μm).

Cytotoxicity Assessment. Cytotoxic potential of the processed nerve cap-graft grafts was assessed using a Neutral Red Uptake (NRU) assay per manufacturer instructions (K447, Biovision). Briefly, nerve cap-graft grafts (n=10) were incubated in conical tubes filled with DMEM (10013CV, Corning) with 10% FBS (35016CV, Corning) and 1% Penicillin/Streptomycin (15140122, Gibco) as extraction media at 37° C. for 48 hours with a tissue surface-to-extraction media ratio of 3 cm2/mL. Schwann cells (Rat, RT4-D6P2T, ATCC CRL-2768) were separately plated at a density of 10,000 cells/well in a 96-well plate and incubated in DMEM (10013CV, Corning) with 10% FBS (35016CV, Corning) and 1% Penicillin/Streptomycin (15140122, Gibco) at 37° C., 5% CO2 overnight. Cell culture media was then aspirated from the wells with cells and replaced with either extraction media, positive control media (cell culture media with 0.05 mM Doxorubicin), negative control media (fresh cell culture media), or vehicle control media (cell culture media incubated for 48 h). Following 24-hour incubation with the test media, cells were washed and exposed to NRU Staining Solution for 2 hours at 37° C., 5% CO2, washed, and then exposed to the Solubilization Solution which caused the viable cells to release the neutral red stain into solution. Light absorbance for the solutions was then measured via microplate reader (800TSI, BioTek) at a wavelength of 540 nm to quantify cell viability. Cells exposed to negative control media were used to normalize results.

Morphology Assessment. Nerve cap-graft graft morphology and socket features were assessed via scanning electron microscopy. Nerve cap-graft grafts were fixed in 2.5% glutaraldehyde overnight while maintained in a straight configuration. Grafts were then dehydrated through a standard critical point drying procedure using a Denton Desk V TSC Sputter Coater. Following critical point drying, grafts (n=3) were sectioned with a scalpel blade in cross section at three points within the socket region of the graft. The samples were then sputter coated with gold and imaged using a scanning electron microscope (JEOL JSM-6510LV/LGS). Four diameter measurements of the socket opening at each cross section were measured using ImageJ. Samples were also sectioned longitudinally, sputter coated, and imaged as described above to visualize the internal aspect of the socket and longitudinal pore region of the grafts.

Mechanical Properties. Nerve cap-graft graft suture retention force was assessed consistent with the methods described in ASTM F3225-17. Briefly, a tensile force test was performed on a uniaxial testing system (Mark-10, ESM303) mounted with a 5 lb. load cell (Mark-10, M5-5 Force Gauge). All nerve cap-graft grafts were tested in a hydrated state at room temperature. Each graft received a single 7-0 nylon suture placed between the outer surface of the graft and the socket lumen. The suture was located 2 mm from the end of the graft through the luminal wall. A loop of 1 cm in diameter was then created with the suture thread and secured with multiple knots to prevent slippage. The distal portion of the graft was secured in vertical position to the lower stage/grip of the uniaxial tensile system and a folded rectangle of 220 grit sandpaper was used to prevent slippage of the sample from the grip. The upper stage of the uniaxial tensile system was mounted with a hook connected to the load cell and then lowered to engage the suture loop tethered to the edge of the graft. The test was performed by pulling the upper stage at the constant rate of 75 mm/min until graft or suture failure. The maximum force (in gf) recorded during the test was considered the suture retention force of the graft.

Implantation Study. Animal studies were performed in accordance with the PHS Policy on Humane Care and Use of Laboratory Animals, the NIH guide for Care and Use of Laboratory Animals, federal and state regulations, and was approved by the Cornell University Institutional Animal Care and Use Committee (IACUC). Upon entrance into the study, animals were acclimated for a period of 7 days prior to any experimental procedures. Record logs of medical procedures were maintained.

Female Lewis rats (200-250 gr) were premedicated with subcutaneous buprenorphine, induced and maintained using inhaled isoflurane in oxygen. Following a standard sterile surgical preparation, an incision in the skin was made at the level of the left biceps femoris. The muscle was then dissected along the connective fascia to expose the sciatic nerve. The left sciatic nerve was transected approximately 5 mm proximal to the bifurcation of the main sciatic branch. A 10 mm nerve segment was then resected proximal to the initial transection to create a gap defect. Gap defects were then repaired using one of two methods: 1) Gap repair (10 mm) with reversed autograft (n=5); 2) Nerve cap-graft graft (10 mm) with connection of the proximal nerve to the proximal portion of the cap, and also distal stump to the distal portion of the graft (n=5). The nerve cap-graft was attached to the distal portion in these experiments to evaluate if the nerve cap-graft arrested axon growth despite the presence of the distal nerve to receive any regenerating axons across the nerve cap-graft. A limited number of animals (n=3) were included in a nerve amputation model in which the distal stump of the transected nerve was ligated before the nerve cap-graft graft (10 mm) was connected to the proximal stump of the transected nerve. An amputation control (n=2) where no nerve cap-graft graft was applied was also included for comparison. All grafts were implanted using 8-0 Nylon monofilament sutures with separate epineural stitches. The nerve cap-graft group in the nerve amputation model was anchored to surrounding muscle tissue adjacent to the distal end of the graft with a 4-0 nylon suture. The muscle and skin were then closed using standard techniques. All animals were survived for 8 weeks following implantation. Following the survival period, the rat sciatic nerves were exposed and electrophysiology was performed on the proximal nerve stump under anesthesia as described below. Animals were then sacrificed and the sciatic nerve and remodeled nerve cap-graft graft were explanted for gross morphologic examination and histologic assessment.

Electrophysiology. Electrophysiologic function was assessed by measurement of compound motor action potential (CMAP) at the gastrocnemius muscle. Briefly, prior to euthanasia and under anesthesia, the sciatic nerve was isolated and two insulated steel electrodes (26g) were placed around the sciatic nerve proximal to the injury and a bipolar concentric recording electrode (Neuroline Concentric 25×0.30 mm, Ambu Inc. MD 21045 Columbia, United States) inserted into the mid body of the gastrocnemius muscle. Supramaximal stimulation was determined by progressively increasing the stimulation current with 0.5 mA increments (100 psec pulse duration) until a plateau was reached. Supramaximal CMAP was then recorded in triplicate and the average calculated for statistical assessment.

Gross Morphology and Histopathology. Sciatic nerve/nerve cap-graft graft samples including a portion of the proximal nerve segment, the nerve cap-graft graft, and a portion of the distal nerve segment were explanted (approximately 1.5 cm in total length) following electrophysiology and euthanasia and placed in a straight configuration on labeled cardstock prior to fixation. Samples were then imaged for gross pathology and immersed in 4% paraformaldehyde. Following 24 hours of fixation, samples were processed and embedded in paraffin blocks and cross-sections of 5 μm in thickness were cut at 2 mm intervals along the longitudinal direction of each sample (3-5 cross-sections per graft), placed on a single slide, and stained with H&E and Luxol Fast Blue (for cellular content and myelin) or Bielschowsky's silver stain (for assessment of axonal morphology). The samples were then evaluated by a board-certified veterinary pathologist with expertise in neuropathology for evidence of nerve regeneration, fibrotic tissue deposition, axonal morphology, and neuroma formation.

Statistical Analysis. Continuous outcome measures were assessed using t-test or ANOVA with Tukey's post hoc tests. All data were analyzed graphically using GraphPad Prism 6 (GraphPad software, Inc). Significance was set as p<0.05 throughout.

Results. Assessment of Decellularization—Histology/Plots. Decellularized nerve cap-graft grafts were found to be largely devoid of myelin. When assessed using a semiquantitative scale (FIGS. 46A and B), the average myelin score for the nerve cap-graft graft was 0.2, while that of the native nerve was 1.8 out of 2 (FIG. 46C). Additional analysis showed that, in both the native and nerve cap-graft graft groups, there was a slight trend toward an increase in myelin score with increased distance into the tissue (FIG. 46C). Few if any nuclei were observed in any of the sections of the nerve cap-graft graft. Representative images of Woelcke's method and H&E staining of native nerve and nerve cap-graft graft are shown in FIG. 46D.

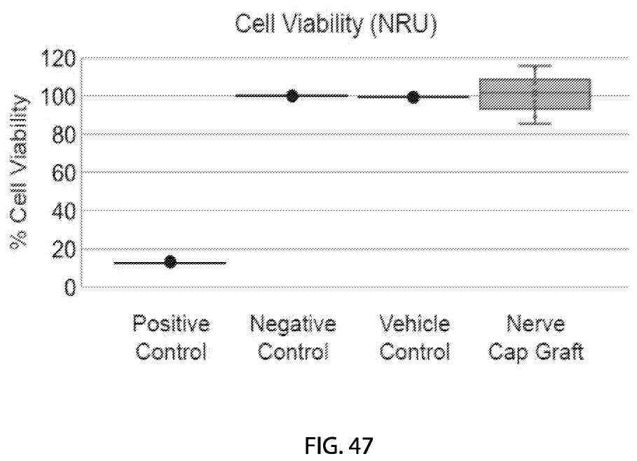

Cytotoxicity—NRU Assay. Results of the cytotoxicity assay demonstrated that extracts of the nerve cap-graft grafts in supplemented media were not cytotoxic (101.0±9.9% cellular viability compared to negative control, FIG. 47). A value of less than 70% in this assay represents significant cytotoxic potential, with positive cytotoxic controls (cell culture media with 0.05 mM Doxorubicin) resulting in 12.5% cellular viability compared to negative control in this assay.

FIG. 47 (Cytotoxicity assay (NRU) showing viability of Schwann cells cultured in control medias or Nerve cap-graft Graft extracts. n=1 sample for positive, negative, and vehicle controls, each performed in sextuplicate; Nerve cap-graft Graft is n=10 samples, each performed in triplicate. Data is presented as box and whisker plot).

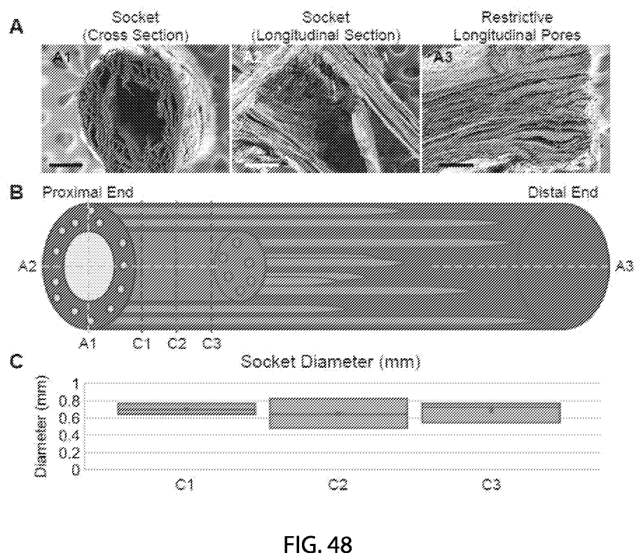

Morphologic Assessment—SEM Imaging. Samples were sectioned to visualize multiple points within the socket of the nerve cap-graft graft in cross section (FIG. 48A1) as well as along the socket (FIG. 48A2) and restrictive longitudinal pore regions (FIG. 48A3) in the longitudinal orientations. The cross sections were characterized by maintenance of the fascicular structure of the nerve tissue; however, axons were removed leaving open spaces within the fascicular structure (corresponding to the longitudinally oriented pores observed in longitudinal sections). The socket of the graft was clearly observed, centered in the cross section of the decellularized nerve tissue. Further analysis of the diameter of the socket was performed showing that the socket had a consistent diameter at each of the levels (FIG. 48B) investigated (C1: 0.7±0.1, C2: 0.6±0.2, C3: C1: 0.7±0.1; FIG. 48C) with no significance (p=0.87) found between groups.

The socket was also clearly observed in the longitudinal orientation (FIG. 48A2). The inner surface of the newly created lumen was fibrous and disorganized, likely as a result of the rotation of the burr used to create the lumen. Well organized, longitudinally oriented pores were observed at the outer aspect of the sample and were shown to continue throughout the remainder of the graft (restrictive longitudinal pore region; FIG. 48A3). In multiple sections, a dense, intact epineural surface was observed covering the outer surface of the graft.

FIG. 48 (SEM imaging and analysis of Nerve cap-graft Graft: A. SEM imaging of Nerve cap-graft Graft features, including the socket (A1 and A2) and restrictive longitudinal pores (A3). Scale=400 μm. B. Representative diagram of Nerve cap-graft Graft with analyzed cross sections (C1, C2, C3). C. Socket diameter analysis of cross section regions. n=3 samples per group, presented as box and whisker plot).

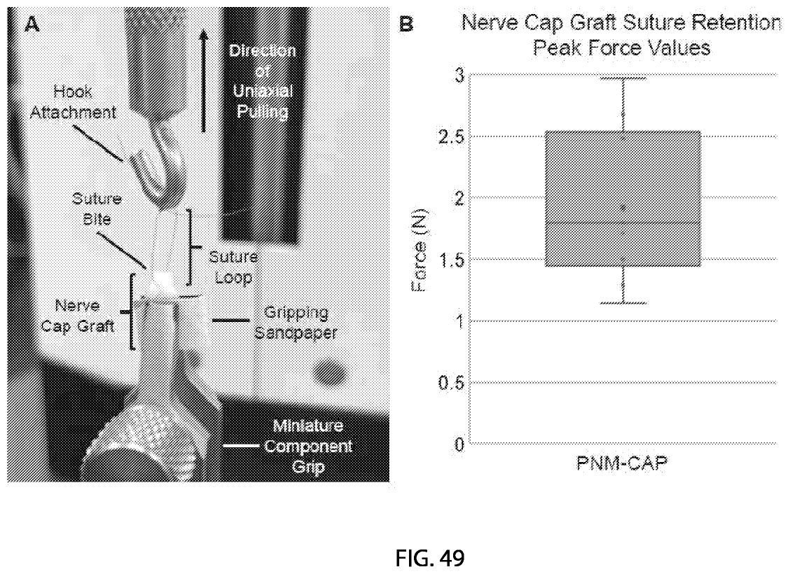

Mechanical Properties—Suture Retention. Nerve cap-graft grafts were assessed for suture retention strength using the setup shown in FIG. 49A. All graft walls (n=10) failed with a peak force between 1.145-2.97 N with an average peak force of 1.93±0.6 N suggesting that the nerve cap-graft grafts had mechanical properties which were suitable for suture in nerve repair applications.

FIG. 49 (Suture retention analysis. A. Suture retention test setup. B. Distribution of peak force values reached prior to Nerve cap-graft Graft wall failure. n=10 samples, presented as box and whisker plot).

Autograft implants were performed by excising a 10 mm segment of the main trunk of the sciatic nerve, inverting the implant, and suturing to the proximal and distal segments. Samples had the appearance of native-like nerve at implant. At 8 weeks post-surgery, grafts appeared well integrated with the proximal and distal segments, with evidence of vascularization. No significant scar tissue or adhesions were observed within the site of implantation. The explanted tissue was opaque white in appearance, suggesting formation of myelin throughout the length of the sample.

The nerve cap-graft graft grafts were successfully attached to both the proximal and distal ends of the sciatic nerve at the time of implantation, with the proximal end of the nerve inserted into socket 2210 of the nerve cap-graft. The diameter of the graft was observed to be greater than that of the peripheral nerve trunk, and the trunk fit well within the socket of the graft. Following implantation of the graft, it was observed that the lumen of the graft was closed around the end of the proximal segment of the sciatic nerve. Upon explantation at 8 weeks, significant remodeling of the graft was observed, with few, if any, remnants of the originally implanted material visible within the site of implantation on macroscopic evaluation. Significant vascularization of the surgical site was observed. No scar tissue or adhesion formation between the graft and surrounding muscle tissues was observed. While both ends of the graft were well integrated with the proximal and distal ends of the sciatic nerve and tissue was observed spanning the gap between the nerve ends, only a small area of opaque white tissue was observed at the proximal aspect of the graft extending into the socket region of the graft, suggesting limited axonal outgrowth into the proximal end of the nerve cap-graft graft.

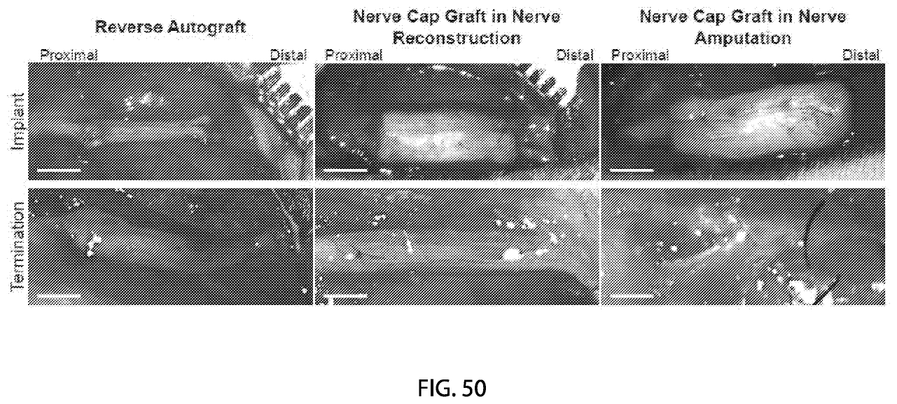

Similar results were observed when the nerve cap-graft graft was attached only to the proximal aspect of the graft without attachment to the distal segment of the nerve. The material was well integrated and vascularized with no evidence of scar tissue or adhesions. Limited axonal ingrowth was evident on gross examination, and no spontaneous reconnection to the distal segment was observed. Representative images of each group at the time of implantation and at explant are shown in FIG. 50.

FIG. 50 (Surgical images of groups at implantation and termination after 8 weeks. Scale=0.25 cm).

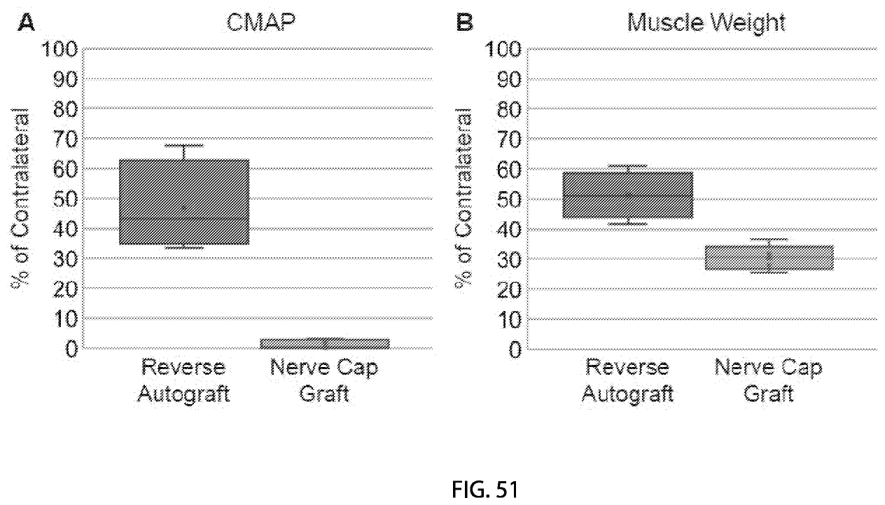

Animal Study—Electrophysiology. Electrophysiology was performed to assess the degree of functional recovery at 8 weeks post-surgically (FIG. 51A). The results showed that the autograft group had a CMAP of 16.6±5.0 mV, corresponding to 46.9±15.0% of the uninjured contralateral side. These values are well within the expected ranges for autograft and demonstrate functional recovery. In contrast, the nerve cap-graft graft group had a CMAP of 0.4±0.5 mV (p=0.0002 when compared to reverse autograft group), corresponding to 1.2±1.5% of the contralateral side (p=0.0002 when compared to the reverse autograft group). These values demonstrate a lack of functional recovery in this group. Electrophysiologic examination was not performed in the nerve cap-graft graft without attachment to the distal stump, as no connection between the proximal and distal segments of the sciatic nerve were observed, and thus no functional activity was expected.

The gastrocnemius of each animal was harvested following electrophysiology and weighed as a secondary measure of functional recovery (FIG. 51B). Results showed that the autograft group had a muscle weight of 0.7±0.07 g, corresponding to 51.2±7.9% of the contralateral side. Similar to the electrophysiologic outcomes, weights of the gastrocnemius in animals treated with the PNM were 0.4±0.06 g (p=0.099 when compared to the reverse autograft group), corresponding to 30.5±4.1% of the contralateral side (p=0.001 when compared to the reverse autograft group). These results suggest increased atrophy in the PNM graft treated animals, which would be expected given the lack of functional recovery observed.

FIG. 51 (Electrophysiology results. A. CMAP results (n=4) compared to contralateral side. B. Gastrocnemius muscle weight (n=5) compared to contralateral side).

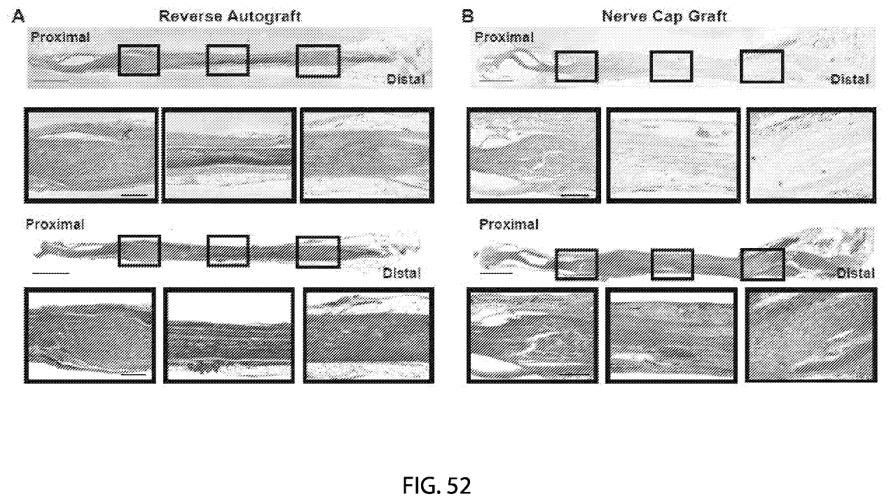

Animal Study—Histology. The reversed autografts (FIG. 52A) were characterized by well aligned axonal regrowth spanning from the proximal to distal ends of the grafts. The axonal regrowth was confined within the autograft with no evidence of axonal misdirection or escape from the graft structure. The axons were found to be well myelinated throughout the length of the graft, which was well integrated with the proximal and distal nerve stumps.

In contrast to the reversed autograft, the nerve cap-graft graft (FIG. 52B) was characterized by well organized, but rapidly dissipating axonal structures within the proximal end of the graft when used to repair a nerve gap injury. As with the autograft, the axons were well aligned, confined to the boundaries of the nerve cap-graft graft, and no axonal escape or evidence of neuroma was noted. Myelin labeling demonstrated well organized, myelinated axons within the proximal stump with little to no myelination at mid graft. The nerve cap-graft grafts were well integrated with the proximal and distal nerve stumps with no evidence of foreign body reaction, rejection, or excessive fibrosis noted on histologic examination and the remodeling grafts were replaced by connective tissue. A prominent angiogenic response was noted along the periphery of the nerve cap-graft grafts. Despite the observed loss of myelin, decrease in axonal number, and replacement of the graft with connective tissue along the longitudinal direction, the linear orientation and organization of the remaining axons in the native nerve and proximal portion of the nerve cap-graft graft was largely maintained. Notably lacking was the presence of aberrant axonal growth producing neuroma-like lesions. These results are consistent with degradation and remodeling of the test article into collagenous connective tissue without significant neuronal outgrowth or neuroma formation.

FIG. 52 (Histologic assessment of autograft and nerve cap-graft graft in gap injury model. A. The reverse autograft is characterized by well-organized axonal outgrowth from the proximal to distal ends of the graft as shown in Bielschowsky's silver stain (top). Similarly, myelination is observed throughout the length of the graft as shown in Luxol fast blue/hematoxylin and eosin stained samples (bottom). B. In contrast, the nerve cap-graft graft is characterized by rapid axonal exhaustion, with few axons shown at mid graft (top). Similarly, myelination is seen only at the healthy, proximal nerve stump, with little staining seen at mid graft. Dark brown staining indicates axons in Bielschosky's silver stain (top), and blue staining indicates myelin in Luxol fast blue/hematoxylin and eosin staining (bottom). Full sample scale bars=2 mm, inset scale bars=500 μm).

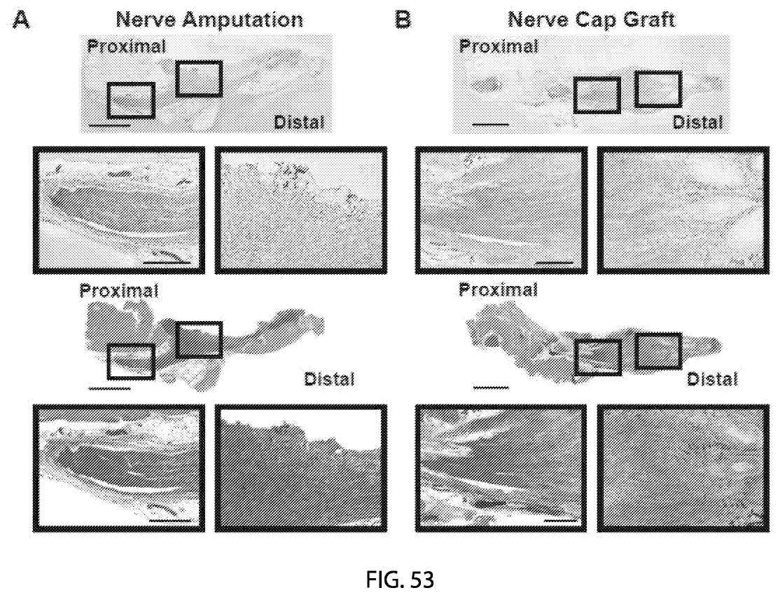

The results for the reversed autograft and nerve cap-graft graft were in contrast to the histopathologic findings for the nerve amputation without repair (neuroma positive control) group (FIG. 53A). The transection without repair group was characterized by a well-organized axonal structure at the proximal aspect of the nerve, transitioning into a tangled array of axons distally at the level of the transection. This area was not protected from the surrounding environment and multiple disorganized and uncontrolled paths of axonal outgrowth were observed. This irregular mass of axons is consistent with what is often seen in neuroma formation.

When the nerve cap-graft graft was used in this model, the observed results were similar to those in the nerve graft model (FIG. 53B). Well organized proximal nerve morphology was observed, transitioning to a well-organized, but axon sparse region at mid graft. There was decreasing myelination observed along the length of the graft from proximal to distal. A small portion of the graft at the distal end was observed to be un-remodeled in some samples. As with the nerve cap-graft grafts implanted in the gap model, axonal growth was contained within the boundaries of the graft, prominent angiogenesis was observed at the periphery of the graft, and no neuroma-like structures were observed.

FIG. 53 (Histologic assessment of untreated and nerve cap-graft graft treated nerve amputation. A. The untreated nerve amputation group is characterized by well-organized axons within the proximal aspect of the amputated nerve, transitioning to an unprotected, disorganized mass of axons at the distal end of the sample. Multiple paths of uncontrolled axonal growth can be observed (top). Similarly, well organized myelination is observed within the proximal aspect of the amputated nerve, with disorganized tissue at the distal aspect of the amputated nerve. B. In contrast, the nerve cap-graft graft is characterized by rapid axonal exhaustion, with a small number of well-organized axons at mid graft (top). An un-remodeled portion of the graft can be observed at the distal third of the sample (inset). Similarly, well organized myelination is seen at the healthy, proximal nerve stump, transitioning to less intense staining at mid graft. Dark brown staining indicates axons in Bielschosky's silver stain (top), and blue staining indicates myelin in Luxol fast blue/hematoxylin and eosin staining (bottom). Full sample scale bars=2 mm, inset scale bars=500 μm).

Discussion. The goal of this study was to develop and test a novel nerve cap-grafting approach entailing an axonal growth restrictive matrix composed of a xenogeneic decellularized nerve graft segment. The nerve cap-graft graft was approximately 1 cm in length, had a defined interface (e.g. socket 2210) for the proximal nerve stump, and longitudinally oriented pores/channels. Decellularization of the nerve cap-graft graft was confirmed via histologic analysis, which showed that the graft was largely devoid of both nuclei and myelin. Cytotoxic assessment of the nerve cap-graft graft showed absence of cytotoxic residual reagents. The nerve cap-graft graft was also assessed for surgical handling and determined to be compatible for use in nerve repair applications. Following implantation in both a nerve gap injury and nerve amputation models, the nerve cap-graft graft appeared to integrate well with the surrounding nerve and muscle tissue without evidence of fibrotic adhesions. Electrophysiology following nerve cap-graft graft implantation after 8 weeks showed complete absence of nerve conduction, indicating the nerve cap-graft graft effectively inhibited nerve axonal regrowth across the nerve injury models used. These results were supported by histopathologic findings, which showed limited, but well-organized, longitudinally-oriented, sparsely myelinated axonal regrowth within the proximal portion of the grafts in a manner distinct from both the well-organized functional regeneration observed in the reversed autograft and the disorganized growth and morphology seen in neuromas.

Neuromas form when insult to the nerve fosters a disorganized growth of axons, which produces a bulbous mass of nerve and scar tissue. In the absence of a guiding substrate, as occurs following nerve transection/amputation, axonal sprouting lacks appropriate directional guidance and signaling cues, leading to a disorganized and unregulated growth (neuroma formation). Histologically, neuroma formation is identifiable as early as at 28 days after injury in rodents.

The present exploratory study utilized a porcine decellularized nerve extracellular matrix structure applied to the proximal stump of the rat sciatic nerve following transection in both gap and nerve amputation models. The device was designed with a socket to host the proximal nerve stump and prevent axonal escape at the nerve-device interface. The main working hypothesis of the present study was that the application of a longitudinally-oriented, but complex and restrictive, guiding structure would allow limited and controlled axonal outgrowth, effectively inhibiting the formation of neuroma. This approach could offer multiple advantages over existing clinical methods for neuroma prevention.

While there is a wide range of preventative solutions for neuromas, results are highly variable across these solutions and there is no clinical ‘gold standard’ to obtain satisfactory results. Simple nerve excision (as is shown in FIG. 53A) is often unsuccessful, as it leaves the new nerve stump surface exposed to the biological and physical inflammatory stimuli naturally present at the area of surgical access, and is associated with high rates of recurrence. Traction neurectomy is more frequently used as it allows the proximal nerve stump to retract into the soft tissue away from the area of surgical access/injury affected by inflammatory stimuli. Yet, traction neurectomy is a mechanical rupture of normal or, worse, a sensitized nerve; this mechanism also occurs in traumatic injuries of nerves and is a known source for neuropathic pain. As an alternative, transposition of the nerve stump into muscle, bone, or a vein is often preferred. This approach provides a dedicated space where nerve regeneration can occur; however, the lack of a directional structural template still exposes the nerve to the risk of disorganized axonal outgrowth or aberrant reinnervation. Additionally, more complex surgical approaches have been used to create new functional interfaces between nerve and muscle tissue in amputees (e.g. targeted muscle reinnervation, regenerative peripheral nerve interface), reducing the rate of neuroma and enabling control of muscles and external prostheses; however, these approaches are highly specialized microsurgical procedures and require use of different surgical expertise, when, ideally, neuromas should be prevented by the vascular or trauma surgeons performing the amputation.

Commercially available nerve cap-grafting devices made of synthetic or biological degradable materials are intended to provide protection from the tissue injury inflammatory environment. While these devices can provide a macrostructural template to contain axonal regeneration, they can also create nerve ‘compartment syndrome’ as they can become a ‘barrier/trap’ for growing/sprouting axons. Furthermore, the degradation products of the synthetic material used for one of these technologies (PLLA-PCA) has been associated with a fibrotic inflammatory response, which can further increase the risk of neuroma formation. These devices have not been found to be more effective than nerve burial approaches, and their clinical adoption has been limited due to insufficient human data.

Prior studies in rat showed that long, decellularized nerve allografts applied to a free nerve ending arrested the formation of neuroma by exhausting the nerve regenerative potential within a length of between 2.5 and 5 cm over the course of 5 weeks and 5 months. While this promising approach benefits from both a protective environment and the longitudinal guidance for sprouting axons at the proximal nerve stump following nerve transection, the use of allografts in the clinic is not ideal due to the shortage of high-quality tissue sources, high variability, and costs associated with cadaveric procurement. Furthermore, these approaches require an extensive length of graft to complete the exhaustion of the native nerve regenerative potential. Such distance requires the creation of an additional surgical pocket, which can increase the complexity and duration of the surgical procedure.

The preliminary approach described here may improve upon the aforementioned limitations of existing approaches by using a degradable ECM material that has been shown to modulate an anti-inflammatory, constructive remodeling profile when used for nerve applications. This nerve cap-graft graft offers a protected and directional path for organized nerve regeneration, with the addition of intrinsically resistive elements for axonal sprouting. Such a combination of features can become an effective approach for neuroma prevention by allowing the development and exhaustion of the nerve regenerative potential, but without requiring extensive lengths, as previously described for the use of acellular nerve allografts. We hypothesize that two elements of our approach could have contributed to the ‘neuro-resistive’ matrix observed in the present studies: 1) the small pore size in the matrix; and 2) the structural complexity mismatch between rat and porcine sciatic nerves. In particular, porcine sciatic nerves have an average of 11-20 fascicles, including small branches (2 mm in diameter), whereas rat sciatic nerves have an average of 3 fascicles.

Despite the promising results observed, this was a preliminary, exploratory study, which included multiple limitations. In particular, the present study focused on a nerve gap injury reconstruction model in lieu of a neuroma formation model, which was tested in a limited number of animals. We justified this choice as nerve gap reconstruction models offer a more challenging environment for demonstrating axonal growth inhibition due to the presence of a contacting distal nerve stump. A sufficiently powered study using a neuroma formation model (without distal stump neurorrhaphy) will be performed in future endeavors to confirm our findings. Furthermore, while neuroma formation has been reported to be visible in rats starting at 28 days, the limited, 56-day, duration of the study may have prevented observation of longer neuronal extensions within the nerve cap-graft graft, and/or neuroma formation over longer periods of time. Therefore, future studies will be extended to a duration of at least 3 months to confirm our findings. Finally, the study did not include standard of care controls for comparison; for example, the study would have benefited from a direct comparison with a nerve burial technique, and/or a commercially available nerve cap-graft device. And finally, the study has not yet compared how this nerve cap-graft graft can impact pain associated with neuroma. The lack of regenerated axons across the nerve cap-graft graft suggests this treatment could reduce pain by shielding these fibers from the wound environment. Further work in this area, including analysis of pain-associated gene expression and the presence of pain-related behaviors could help answer this question. Such comparisons will be explored in future studies.

In conclusion, the present exploratory study, while limited in scope, provides proof of concept that a xenogeneic acellular nerve cap-graft graft can be used to prevent neuroma formation following nerve transection injury. This could be a promising alternative approach for neuroma prevention in applications where nerve functional restoration is not attainable or desirable.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the present inventive concepts, which are defined in the accompanying claims.

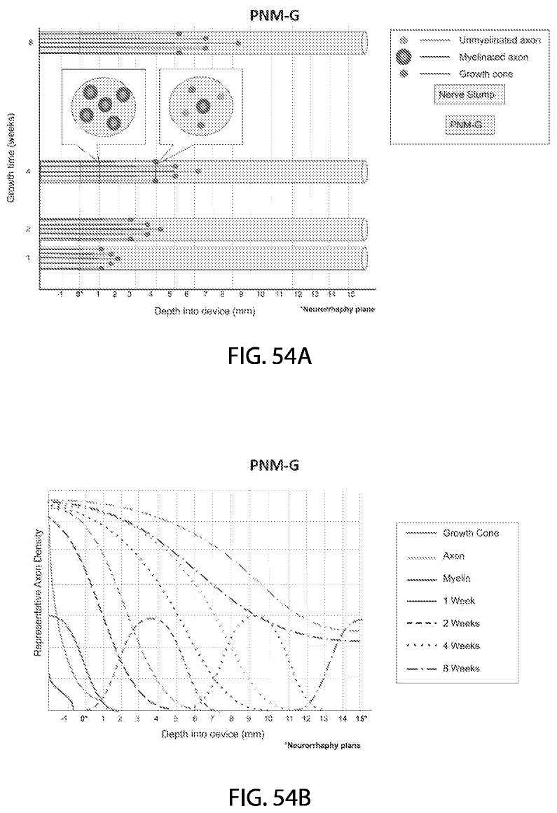

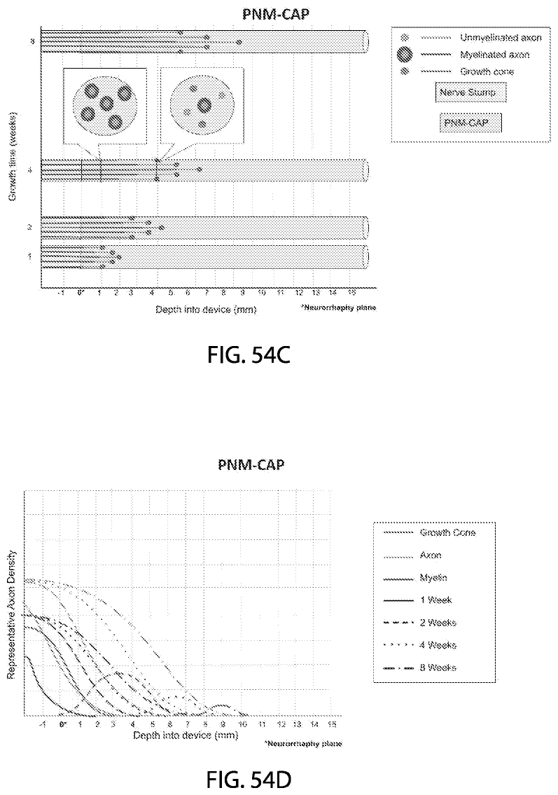

Referring now to FIGS. 54A-D, graphical representations of axon infiltration into a nerve-graft conduit (e.g. PNM-CAP 2200, PNM-G 2100) and the relative location of axon growth cones, unmyelinated axons, and myelinated axons are illustrated, consistent with the present inventive concepts. In some embodiments, the device constitutes a neuroinductive matrix contained within a nerve graft structure (PNM-G 2100). PNM-G 2100 can be sutured to a peripheral nerve stump at one end and a distal nerve stump at the opposite end. PNM-G 2100 can facilitate the growth of axons from the peripheral nerve stump through to the distal nerve stump. FIG. 54A illustrates a representative front of axon progression through PNM-G 2100 to the distal nerve stump at 1, 2, 4, and 8 weeks. Axons grow at a relatively equal rate, resulting in growth cone presence at similar depth for each time point. Additionally, myelin is present around axons at a uniform depth difference from the growth cone, consistent with the myelination of healthy growing axons. Representative cross-sections at the 4-week timepoint further illustrate the relative positioning of growth cones, myelinated axons, and unmyelinated axons within PNM-G 2100. A graphical model of the representative axon growth in FIG. 54A through PNM-G 2100 is illustrated in FIG. 54B. Axon growth cones move through the device in a wave front of uniform distribution. Axon density at the neurorrhaphy plane approaches a constant over time, which is consistent with the axonal density of the peripheral nerve stump. With increasing depth, the axon density decreases, and the slope of this decline becomes less steep over time as more axons infiltrate further into the device. The axon density approaches a plateau of constant density across the device, which represents the time at which there are no growth cones within PNM-G 2100 and the axons are fully formed across the gap injury into the distal stump. The myelin density follows the trend of axon density with a time shift to account for the region of unmyelinated axons immediately following the growth cone.

In some embodiments, the device constitutes a neuroinhibitory matrix contained within a cap structure (PNM-CAP 2200), which is sutured to a peripheral nerve stump in order to prevent neuroma formation. PNM-CAP 2200 can slow and eventually stop the growth of axons from the peripheral nerve stump before the end of the neuroinhibitory matrix. FIG. 54C illustrates a representative front of axon progression through PNM-CAP 2200 at 1, 2, 4, and 8 weeks. As depth into PNM-CAP 2200 decreases, the front becomes less uniform, indicating that some axons growth has been slowed or stopped. Additionally, myelination of axons decreases as axons progress into the matrix, resulting in a greater length of unmyelinated axons at later timepoints. Representative cross-sections at the 4-week timepoint further illustrate the relative positioning of growth cones, myelinated axons, and unmyelinated axons within PNM-CAP 2200. A graphical model of the representative axon growth in FIG. 54C through PNM-CAP 2200 is illustrated in FIG. 54D. Growth cones move through the device in a wave front. Unlike the uniform peaks of PNM-G 2100, the neuroinhibitory nature of the PNM-CAP 2200 matrix causes both the axon density of each front and the distance between fronts to decrease over time as axon growth slows. Axon density at the neurorrhaphy plane approaches a constant over time. At further depth, the axon density decreases rapidly, although the slope of this decline becomes less steep over time as more axons infiltrate further into the device. Axon density does not extend the entire length of the device. The myelin density follows the trend of axon density; however, the myelin is a consistently lower density, and does not penetrate to the same depth into the device as the axon growth.

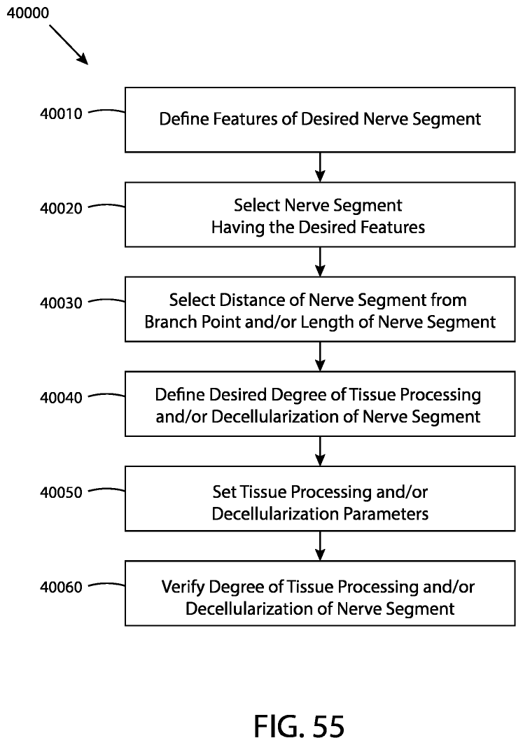

Referring now to FIG. 55, a method for obtaining ideal decellularization in order to obtain desired structure and function in both the nerve graft-conduit or nerve cap-graft, consistent with the present inventive concepts Method 40000 can be configured to customize the choice of raw material 65. Method 40000 can be configured to obtain an ideal (e.g. preferred) degree of decellularization in different sized nerve segments. It has been previously demonstrated that decellularization decreases with radial depth of the nerve graft-conduit (e.g. PNM-G 2100) or nerve cap-graft (e.g. PNM-CAP 2200). Thus, a nerve segment comprising a larger diameter requires longer incubation period in detergent solution to obtain the same level of decellularization as a nerve segment comprising a small diameter. In addition to diameter, fascicular complexity can impact the degree of decellularization. As previously discussed, a nerve graft-conduit (e.g. PNM-G 2100) or nerve cap-graft (e.g. PNM-CAP 2200) can be selected from a specific nerve branch at a specific distance from the proximal branching point, which can result in a graft with defined features (e.g. external diameter and fascicular complexity). Based on these parameters and the degree of decellularization desired for the target application, the decellularization process can be tailored to each individual nerve segment to produce equally decellularized grafts with different features (e.g. external diameter and fascicular complexity).

In some embodiments, a graft (e.g. PNM-G 2100, PNM-CAP 2200) can comprise a nerve segment from the main sciatic trunk comprising a large diameter and high fascicular number. This nerve segment can require a longer incubation in detergent solution, such as an incubation period of 10 hours. In some embodiments, a graft (e.g. PNM-G 2100, PNM-CAP 2200) can comprise a nerve segment from the sural branch of the sciatic nerve comprising a smaller diameter and low fascicular number. This nerve segment can undergo a shorter incubation in detergent solution, such as an incubation period of 6 hours. To supplement this approach to decellularization, the cellular content in the detergent solution may be monitored through one, two, or more features, such as turbidity, absorbance, conductivity, and pH.

In STEP 40010, one, two, or more features of a desired nerve segment are defined. The external diameter, length, and/or structure/fascicular complexity of the desired nerve segment can be defined based on the intended use. For example, if a graft (e.g. PNM-CAP 2200) is intended to cap a nerve stump comprising a diameter of 3 mm in diameter with the goal of preventing neuroma formation, the graft would be derived from a nerve segment sourced from anatomic locations of similar external diameter. The intended use as PNM-CAP 2200 can also inform the structure/fascicular complexity of the nerve segment source. In some embodiments, it is desirable to select a nerve segment from an anatomic location characterized by high fascicular complexity.

In STEP 40020, a nerve segment having one, two, or more the desired features as defined in STEP 40010 is selected. In some embodiments, the source nerve segment would be selected from a nerve branch and location known to possess at least one of the desired features.

In STEP 40030, the nerve segment as selected in STEP 40020, is further selected to begin at a known distance from a branching point (e.g. a proximal branching point). In some embodiments, the nerve segment location is selected to comprise at least one desired feature and/or to allow sufficient a graft length.

In STEP 40040, a desired degree of tissue processing and/or decellularization of the nerve segment for a target application is defined. For example, if a graft is intended to cap a nerve stump with the goal of preventing neuroma formation, the nerve segment can be processed and/or decellularized to a degree (e.g. high degree) configured to completely remove adhesive molecules, such as laminin, which is known to support nerve regeneration. As another example, if a graft is intended to bridge a nerve defect with the goal of inducing nerve regeneration through the graft, the nerve segment can be processed and/or decellularized to a degree (e.g. low degree) configured to preserve adhesive molecules, such as laminin.

In STEP 40050, one, two, or more tissue processing and/or decellularization parameters are set. In some embodiments, the tissue processing and/or decellularization parameters include the incubation time, the temperature, the concentration of the reagent use, the degree of washing following treatment with the reagents, etc.

In STEP 40060, the degree of tissue processing and/or decellularization of the nerve segment can be analyzed during and/or following the tissue processing and/or decellularization to confirm that the desired features have been obtained. For example, the degree of tissue processing can be measured and/or otherwise tracked during the process by measuring the absorbance to visible light through the solution used for decellularization and/or the solution used for washing the tissue following tissue processing. The degree of tissue processing and/or decellularization and the structural and fascicular complexity of the nerve segment can be also confirmed by performing histological analysis of a sample of the nerve segment. The diameter and length of the nerve segment can be confirmed with standard tools after completion of the process.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

1. A nerve cap-graft system comprising:
at least one of a nerve graft-conduit or nerve cap-graft comprising a peripheral nerve tissue derived from a tissue source; the peripheral nerve tissue being treated such that the peripheral nerve tissue is configured to inhibit nerve growth and/or neuroma formation,
wherein the system provides a therapeutic benefit to a patient.

2. The system according to claim 1, wherein the at least one nerve graft-conduit or nerve cap-graft is configured to be remodeled over time into a native tissue of the patient.

3. (canceled)

4. The system according to claim 1, wherein the treated peripheral nerve tissue comprises a decellularized extracellular matrix.

5. The system according to claim 1, wherein the decellularized extracellular matrix comprises structural and/or non-structural biomolecules, including endogenous factors.

6. (canceled)

7. (canceled)

8. (canceled)

9. (canceled)

10. (canceled)

11. (canceled)

12. The system according to claim 1, wherein the tissue source is selected and harvested from a specific animal species of a specific age, sex, and/or weight.

13. (canceled)

14. The system according to claim 1, wherein the at least one nerve graft-conduit or nerve cap-graft is designed and/or manufactured with one, two, or more geometrical, structural, and functional qualities intended to match or mismatch the geometrical, structural, and functional qualities of a nerve site in the patient.

15. The system according to claim 1, wherein the at least one nerve graft-conduit or nerve cap-graft comprise different degrees of tissue processing and/or decellularization.

16. (canceled)

17. (canceled)

18. (canceled)

19. The system according to claim 1, wherein the nerve cap-graft is configured to be at least partially placed over one, two, or more nerve endings.

20. The system according to claim 1, wherein the at least one nerve graft-conduit or nerve cap-graft is constructed and arranged as a nerve connector configured to align and/or connect one, two or more nerve endings.

21. (canceled)

22. (canceled)

23. (canceled)

24. The system according to claim 1, wherein the treated peripheral nerve tissue is configured to control one or more design variables selected from the group consisting of: porosity; pore size; pore interconnectedness; pore alignment; degradation rate; swell ratio; and combinations thereof.

25. A method for producing a nerve cap graft, the method comprising:
harvesting and/or preparing a nerve segment;
chemically, physically, and/or mechanically treating the harvested nerve segment to decellurlarize it;
providing external support to the treated nerve segment;
freezing the externally supported treated nerve segment;
lyophilizing the externally supported treated nerve segment;
creating one, two, or more desired features in the lyophilized treated nerve segment;
chemically, physically, and/or mechanically treating the lyophilized nerve segment;
stabilizing the treated lyophilized nerve segment;
packaging the lyophilized treated nerve segment;
sterilizing the packaged lyophilized treated nerve segment; and
shipping and/or storing the packaged lyophilized nerve segment.

26. (canceled)

27. A method for treating a patient comprising:
deploying a device comprising at least one of a nerve graft-conduit or nerve cap-graft at a deposit site in the patient,
wherein the device is configured to provide a therapeutic benefit at a treatment site.

28. (canceled)

29. (canceled)

30. (canceled)

31. (canceled)

32. The system according to claim 1, wherein the nerve cap-graft comprises one or more of the flowing features: a socket, neuro-inhibitory matrix, and an anchoring tab.

33. The system according to claim 1, wherein the socket is configured to accept, protect, and anchor a nerve ending of a specific size range.

34. The system according to claim 1, wherein the anchoring tab is configured to allow connection to of the nerve cap-graft and the anchored nerve ending to surrounding tissues.

35. A method for obtaining neuro-inhibitory properties and/or features within a nerve cap-graft, comprising a controlled tissue compression obtained via an external support system.

36. A method for obtaining at least one nerve cap-graft feature, comprising a removal of nerve cap-graft material via laser ablation.

37. The system according to claim 1, wherein the peripheral nerve tissue is treated to remove potential immunogenic components such as cellular components and myelin.

38. The system according to claim 1, wherein the at least one nerve graft conduit or nerve cap-graft degrades in vivo with a degradation rate of between 2 weeks and 6 months.
